(19) United States
(12) Patent Application Publication
(10) Pub. No.: US 2009/0320165 A1
(43) Pub. Date: Dec. 24, 2009

(54) MODULATION OF PROTEIN LEVELS IN PLANTS

(76) Inventors: Steven Craig Bobzin, Malibu, CA (US); Daniel Mumenthaler, Bonita, CA (US); Joel Cruz Rarang, Granada Hills, CA (US)

Correspondence Address: FISH & RICHARDSON P.C., PO BOX 1022, MINNEAPOLIS, MN, 55440-1022, US

(21) Appl. No.: 12/305,282

(22) PCT Filed: Jun. 21, 2007
(86) PCT No.: PCT/US07/14617

§ 371 Date: Jun. 29, 2009

Related U.S. Application Data
(60) Provisional application No. 60/815,535, filed on Jun. 21, 2006.

(51) Int. Cl. A01H 5/00; C12N 5/04; A01H 5/10; A01H 5/08; C07H 21/00

(52) U.S. Cl. 800/298; 435/419; 435/415; 435/416; 435/412; 536/23.1

(57) ABSTRACT

Methods and materials for modulating, e.g., increasing or decreasing, protein levels in plants are disclosed. For example, nucleic acids encoding protein-modulating polypeptides are disclosed as well as methods for using such nucleic acids to transform plant cells. Also disclosed are plants having increased protein levels and plant products produced from plants having increased protein levels.

### BACKGROUND

1. Technical Field

This document relates to methods and materials involved in modulating (e.g., increasing or decreasing) protein levels in plants. For example, this document provides plants having increased protein levels as well as materials and methods for making plants and plant products having increased protein levels.

2. Incorporation-by-Reference & Texts

The material on the accompanying diskette is hereby incorporated by reference into this application. The accompanying three compact discs all contain one identical file, Sequence Listing 11696-228WO1.txt, which was created on Jun. 21, 2007. The file named 11696-228WO1.txt is 837 KB. The file can be accessed using Microsoft Word on a computer that uses Windows OS.

3. Background Information

Protein is an important nutrient required for growth, maintenance, and repair of tissues. The building blocks of proteins are 20 amino acids that may be consumed from both plant and animal sources. Most microorganisms such as E. coli can synthesize the entire set of 20 amino acids, whereas human beings cannot make nine of them. The amino acids that must be supplied in the diet are called essential amino acids, whereas those that can be synthesized endogenously are termed nonessential amino acids. These designations refer to the needs of an organism under a particular set of conditions. For example, enough arginine is synthesized by the urea cycle to meet the needs of an adult, but perhaps not those of a growing child. A deficiency of even one amino acid results in a negative nitrogen balance. In this state, more protein is degraded than is synthesized, and so more nitrogen is excreted than is ingested.

According to U.S. government standards, the Recommended Daily Allowance (RDA) of protein is 0.8 gram per kilogram of ideal body weight for the adult human. The biological value of a dietary protein is determined by the amount and proportion of essential amino acids it provides. If the protein in a food supplies all of the essential amino acids, it is called a complete protein. If the protein in a food does not supply all of the essential amino acids, it is designated as an incomplete protein. Meat and other animal products are sources of complete proteins. However, a diet high in meat can lead to high cholesterol or other diseases, such as gout. Some plant sources of protein are considered to be partially complete because, although consumed alone they may not meet the requirements for essential amino acids, they can be combined to provide amounts and proportions of essential amino acids equivalent to those in proteins from animal sources. Soy protein is an exception because it is a complete protein. Soy protein products can be good substitutes for animal products because soybeans contain all of the amino acids essential to human nutrition and they have less fat, especially saturated fat, than animal-based foods. The U.S. Food and Drug Administration (FDA) determined that diets including four daily soy servings can reduce levels of low-density lipoproteins (LDLs), the cholesterol that builds up in blood vessels, by as much as 10 percent (Henkel, FDA Consumer, 34:3 (2000); fda.gov/fdac/features/2000/300_soy.html). FDA allows a health claim on food labels stating that a daily diet containing 25 grams of soy protein, that is also low in saturated fat and cholesterol, may reduce the risk of heart disease (Henkel, FDA Consumer, 34:3 (2000); fda.gov/fdac/features/2000/300_soy.html).

There is a need for methods of increasing protein production in plants, which provide healthier and more economical sources of protein than animal products.

### SUMMARY

This document provides methods and materials related to plants having modulated (e.g., increased or decreased) levels of protein. For example, this document provides transgenic plants and plant cells having increased levels of protein, nucleic acids used to generate transgenic plants and plant cells having increased levels of protein, and methods for making plants and plant cells having increased levels of protein. Such plants and plant cells can be grown to produce, for example, seeds having increased protein content. Seeds having increased protein levels may be useful to produce foodstuffs and animal feed having increased protein content, which may benefit both food producers and consumers.

In one aspect, a method of modulating the level of protein in a plant is provided. The method comprises introducing into a plant cell an isolated nucleic acid comprising a nucleotide sequence encoding a polypeptide having 80 percent or greater sequence identity to an amino acid sequence selected from the group consisting of SEQ ID NOs:80-82, SEQ ID NOs:84-93, SEQ ID NOs:95-96, SEQ ID NOs:98-100, SEQ ID NOs:102-103, SEQ ID NO:105, SEQ ID NO:107, SEQ ID NOs:109-110, SEQ ID NO:112, SEQ ID NO:114, SEQ ID NOs:116-117, SEQ ID NOs:119-122, SEQ ID NOs:124-128, SEQ ID NO:130, SEQ ID NOs:132-133, SEQ ID NOs:135-139, SEQ ID NOs:141-150, SEQ ID NO:152, SEQ ID NOs:154-155, SEQ ID NOs:157-159, SEQ ID NOs:161-162, SEQ ID NO:164, SEQ ID NOs:166-169, SEQ ID NO:171, SEQ ID NO:173, SEQ ID NOs:175-178, SEQ ID NO:180, SEQ ID NOs:182-187, SEQ ID NO:189, SEQ ID NOs:191-196, SEQ ID NOs:198-203, SEQ ID NO:205, SEQ ID NO:209, SEQ ID NOs:211-212, SEQ ID NOs:214-215, SEQ ID NOs:217-218, SEQ ID NO:220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NOs:248-250, SEQ ID NO:252, SEQ ID NO:254, SEQ ID NOs:256-286, SEQ ID NO:315, SEQ ID NOs:317-328, SEQ ID NOs:330, SEQ ID NO:332, SEQ ID NO:334, SEQ ID NOs:336-337, SEQ ID NO:339, SEQ ID NO:341, and SEQ ID NOs:343-349, where a tissue of a plant produced from the plant cell has a difference in the level of protein as compared to the corresponding level in tissue of a control plant that does not comprise the nucleic acid.

In another aspect, a method of modulating the level of protein in a plant is provided. The method comprises introducing into a plant cell an isolated nucleic acid comprising a nucleotide sequence having 80 percent or greater sequence identity to a nucleotide sequence corresponding to SEQ ID NO:206, where a tissue of a plant produced from the plant cell has a difference in the level of protein as compared to the corresponding level in tissue of a control plant that does not comprise the nucleic acid.

The sequence identity can be 85 percent or greater, 90 percent or greater, or 95 percent or greater. The nucleotide sequence can encode a polypeptide comprising an amino acid sequence corresponding to SEQ ID NO:80. The nucleotide sequence can encode a polypeptide comprising an amino acid sequence corresponding to SEQ ID NO:84. The nucleotide sequence can encode a polypeptide comprising an amino acid sequence corresponding to SEQ ID NO:95. The nucleotide sequence can encode a polypeptide comprising an amino acid sequence corresponding to SEQ ID NO:102. The nucleotide sequence can encode a polypeptide comprising an amino acid sequence corresponding to SEQ ID NO:112. The nucleotide sequence can encode a polypeptide comprising an amino acid sequence corresponding to SEQ ID NO:114. The nucleotide sequence can encode a polypeptide comprising an amino acid sequence corresponding to SEQ ID NO:119. The nucleotide sequence can encode a polypeptide comprising an amino acid sequence corresponding to SEQ ID NO:130. The nucleotide sequence can encode a polypeptide comprising an amino acid sequence corresponding to SEQ ID NO:141. The nucleotide sequence can encode a polypeptide comprising an amino acid sequence corresponding to SEQ ID NO:161. The nucleotide sequence can encode a polypeptide comprising an amino acid sequence corresponding to SEQ ID NO:171. The nucleotide sequence can encode a polypeptide comprising an amino acid sequence corresponding to SEQ ID NO:175. The nucleotide sequence can encode a polypeptide comprising an amino acid sequence corresponding to SEQ ID NO:180. The nucleotide sequence can encode a polypeptide comprising an amino acid sequence corresponding to SEQ ID NO:182. The nucleotide sequence can encode a polypeptide comprising an amino acid sequence corresponding to SEQ ID NO:191. The nucleotide sequence can encode a polypeptide comprising an amino acid sequence corresponding to SEQ ID NO:205. The nucleotide sequence can encode a polypeptide comprising an amino acid sequence corresponding to SEQ ID NO:209. The nucleic acid can comprise a nucleotide sequence corresponding to SEQ ID NO:206. The difference can be an increase in the level of protein. The isolated nucleic acid can be operably linked to a regulatory region. The regulatory region can be a promoter. The promoter can be a tissue-preferential, broadly expressing, or inducible promoter. The plant can be a dicot. The plant can be a species selected from the group consisting of Beta vulgaris (sugarbeet), Brassica napus (canola), Glycine max (soybean), Helianthus annuus (sunflower), Lupinus albus (lupin), or Medicago saliva (alfalfa). The plant can be a monocot. The plant can be a species selected from the group consisting of Oryza sativa (rice), Pennisetum glaucum (pearl millet), Triticum aestivum, (wheat), or Zea mays (corn).

A method of producing a plant tissue is also provided. The method comprises growing a plant cell comprising an exogenous nucleic acid comprising a nucleotide sequence encoding a polypeptide having 80 percent or greater sequence identity to an amino acid sequence selected from the group consisting of SEQ ID NOs:80-82, SEQ ID NOs:84-93, SEQ ID NOs:95-96, SEQ ID NOs:98-100, SEQ ID NOs:102-103, SEQ ID NO:105, SEQ ID NO:107, SEQ ID NOs:109-110, SEQ ID NO:112, SEQ ID NO:114, SEQ ID NOs:116-117, SEQ ID NOs:119-122, SEQ ID NOs:124-128, SEQ ID NO:130, SEQ ID NOs:132-133, SEQ ID NOs:135-139, SEQ ID NOs:141-150, SEQ ID NO:152, SEQ ID NOs:154-155, SEQ ID NOs:157-159, SEQ ID NOs:161-162, SEQ ID NO:164, SEQ ID NOs:166-169, SEQ ID NO:171, SEQ ID NO:173, SEQ ID NOs:175-178, SEQ ID NO:180, SEQ ID NOs:182-187, SEQ ID NO:189, SEQ ID NOs:191-196, SEQ ID NOs:198-203, SEQ ID NO:205, SEQ ID NO:209, SEQ ID NOs:211-212, SEQ ID NOs:214-215, SEQ ID NOs:217-218, SEQ ID NO:220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NOs:248-250, SEQ ID NO:252, SEQ ID NO:254, SEQ ID NOs:256-286, SEQ ID NO:315, SEQ ID NOs:317-328, SEQ ID NOs:330, SEQ ID NO:332, SEQ ID NO:334, SEQ ID NOs:336-337, SEQ ID NO:339, SEQ ID NO:341, and SEQ ID NOs:343-349, where the tissue has a difference in the level of protein as compared to the corresponding level in tissue of a control plant that does not comprise the nucleic acid.

In another aspect, a method of producing a plant tissue is provided. The method comprises growing a plant cell comprising an exogenous nucleic acid comprising a nucleotide sequence having 80 percent or greater sequence identity to a nucleotide sequence corresponding to SEQ ID NO:206, where the tissue has a difference in the level of protein as compared to the corresponding level in tissue of a control plant that does not comprise the nucleic acid.

The sequence identity can be 85 percent or greater, 90 percent or greater, or 95 percent or greater. The nucleotide sequence can encode a polypeptide comprising an amino acid sequence corresponding to SEQ ID NO:80. The nucleotide sequence can encode a polypeptide comprising an amino acid sequence corresponding to SEQ ID NO:84. The nucleotide sequence can encode a polypeptide comprising an amino acid sequence corresponding to SEQ ID NO:95. The nucleotide sequence can encode a polypeptide comprising an amino acid sequence corresponding to SEQ ID NO:102. The nucleotide sequence can encode a polypeptide comprising an amino acid sequence corresponding to SEQ ID NO:112. The nucleotide sequence can encode a polypeptide comprising an amino acid sequence corresponding to SEQ ID NO:114. The nucleotide sequence can encode a polypeptide comprising an amino acid sequence corresponding to SEQ ID NO:119. The nucleotide sequence can encode a polypeptide comprising an amino acid sequence corresponding to SEQ ID NO:130. The nucleotide sequence can encode a polypeptide comprising an amino acid sequence corresponding to SEQ ID NO:141. The nucleotide sequence can encode a polypeptide comprising an amino acid sequence corresponding to SEQ ID NO:161. The nucleotide sequence can encode a polypeptide comprising an amino acid sequence corresponding to SEQ ID NO:171. The nucleotide sequence can encode a polypeptide comprising an amino acid sequence corresponding to SEQ ID NO:175. The nucleotide sequence can encode a polypeptide comprising an amino acid sequence corresponding to SEQ ID NO:180. The nucleotide sequence can encode a polypeptide comprising an amino acid sequence corresponding to SEQ ID NO:182. The nucleotide sequence can encode a polypeptide comprising an amino acid sequence corresponding to SEQ ID NO:191. The nucleotide sequence can encode a polypeptide comprising an amino acid sequence corresponding to SEQ ID NO:205. The nucleotide sequence can encode a polypeptide comprising an amino acid sequence corresponding to SEQ ID NO:209. The exogenous nucleic acid can comprise a nucleotide sequence corresponding to SEQ ID NO:206. The difference can be an increase in the level of protein. The exogenous nucleic acid can be operably linked to a regulatory region. The regulatory region can be a promoter. The promoter can be a tissue-preferential, broadly expressing, or inducible promoter. The plant tissue can be dicotyledonous. The plant tissue can be a species selected from the group consisting of Beta vulgaris (sugarbeet), Brassica napus (canola), Glycine max (soybean), Helianthus annuus (sunflower), Lupinus albus (lupin), or Medicago saliva (alfalfa). The plant tissue can be monocotyledonous. The plant tissue can be a species selected from the group consisting of Oryza saliva (rice), Pennisetum glaucum (pearl millet), Triticum aestivum, (wheat), or Zea mays (corn).

A plant cell is also provided. The plant cell comprises an exogenous nucleic acid comprising a nucleotide sequence encoding a polypeptide having 80 percent or greater sequence identity to an amino acid sequence selected from the group consisting of SEQ ID NOs:80-82, SEQ ID NOs:84-93, SEQ ID NOs:95-96, SEQ ID NOs:98-100, SEQ ID NOs:102-103, SEQ ID NO:105, SEQ ID NO:107, SEQ ID NOs:109-110, SEQ ID NO:112, SEQ ID NO:114, SEQ ID NOs:116-117, SEQ ID NOs:119-122, SEQ ID NOs:124-128, SEQ ID NO:130, SEQ ID NOs:132-133, SEQ ID NOs:135-139, SEQ ID NOs:141-150, SEQ ID NO:152, SEQ ID NOs:154-155, SEQ ID NOs:157-159, SEQ ID NOs:161-162, SEQ ID NO:164, SEQ ID NOs:166-169, SEQ ID NO:171, SEQ ID NO:173, SEQ ID NOs:175-178, SEQ ID NO:180, SEQ ID NOs:182-187, SEQ ID NO:189, SEQ ID NOs:191-196, SEQ ID NOs:198-203, SEQ ID NO:205, SEQ ID NO:209, SEQ ID NOs:211-212, SEQ ID NOs:214-215, SEQ ID NOs:217-218, SEQ ID NO:220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NOs:248-250, SEQ ID NO:252, SEQ ID NO:254, SEQ ID NOs:256-286, SEQ ID NO:315, SEQ ID NOs:317-328, SEQ ID NOs:330, SEQ ID NO:332, SEQ ID NO:334, SEQ ID NOs:336-337, SEQ ID NO:339, SEQ ID NO:341, and SEQ ID NOs:343-349, where a tissue of a plant produced from the plant cell has a difference in the level of protein as compared to the corresponding level in tissue of a control plant that does not comprise the nucleic acid.

In another aspect, a plant cell is provided. The plant cell comprises an exogenous nucleic acid comprising a nucleotide sequence having 80 percent or greater sequence identity to a nucleotide sequence corresponding to SEQ ID NO:206, where a tissue of a plant produced from the plant cell has a difference in the level of protein as compared to the corresponding level in tissue of a control plant that does not comprise the nucleic acid.

The sequence identity can be 85 percent or greater, 90 percent or greater, or 95 percent or greater. The nucleotide sequence can encode a polypeptide comprising an amino acid sequence corresponding to SEQ ID NO:80. The nucleotide sequence can encode a polypeptide comprising an amino acid sequence corresponding to SEQ ID NO:84. The nucleotide sequence can encode a polypeptide comprising an amino acid sequence corresponding to SEQ ID NO:95. The nucleotide sequence can encode a polypeptide comprising an amino acid sequence corresponding to SEQ ID NO:102. The nucleotide sequence can encode a polypeptide comprising an amino acid sequence corresponding to SEQ ID NO:112. The nucleotide sequence can encode a polypeptide comprising an amino acid sequence corresponding to SEQ ID NO:114. The nucleotide sequence can encode a polypeptide comprising an amino acid sequence corresponding to SEQ ID NO:119. The nucleotide sequence can encode a polypeptide comprising an amino acid sequence corresponding to SEQ ID NO:130. The nucleotide sequence can encode a polypeptide comprising an amino acid sequence corresponding to SEQ ID NO:141. The nucleotide sequence can encode a polypeptide comprising an amino acid sequence corresponding to SEQ ID NO:161. The nucleotide sequence can encode a polypeptide comprising an amino acid sequence corresponding to SEQ ID NO:171. The nucleotide sequence can encode a polypeptide comprising an amino acid sequence corresponding to SEQ ID NO:175. The nucleotide sequence can encode a polypeptide comprising an amino acid sequence corresponding to SEQ ID NO:180. The nucleotide sequence can encode a polypeptide comprising an amino acid sequence corresponding to SEQ ID NO:182. The nucleotide sequence can encode a polypeptide comprising an amino acid sequence corresponding to SEQ ID NO:191. The nucleotide sequence can encode a polypeptide comprising an amino acid sequence corresponding to SEQ ID NO:205. The nucleotide sequence can encode a polypeptide comprising an amino acid sequence corresponding to SEQ ID NO:209. The exogenous nucleic acid can comprise a nucleotide sequence corresponding to SEQ ID NO:206. The difference can be an increase in the level of protein. The exogenous nucleic acid can be operably linked to a regulatory region. The regulatory region can be a promoter. The promoter can be a tissue-preferential, broadly expressing, or inducible promoter. The plant can be a dicot. The plant can be a species selected from the group consisting of Beta vulgaris (sugarbeet), Brassica napus (canola), Glycine max (soybean), Helianthus annuus (sunflower), Lupinus albus (lupin), or Medicago sativa (alfalfa). The plant can be a monocot. The plant can be a species selected from the group consisting of Oryza saliva (rice), Pennisetum glaucum (pearl millet), Triticum aestivum, (wheat), or Zea mays (corn). The tissue can be seed tissue.

A transgenic plant is also provided. The transgenic plant comprises any of the plant cells described above. Progeny of the transgenic plant are also provided. The progeny has a difference in the level of protein as compared to the level of protein in a corresponding control plant that does not comprise the isolated nucleic acid. Seed, vegetative tissue, and fruit from the transgenic plant are also provided. In addition, food products and feed products comprising seed, vegetative tissue, and/or fruit from the transgenic plant are provided. Protein from the transgenic plant, which can be a soybean plant, is also provided.

In another aspect, a method of modulating the level of protein in a plant is provided. The method comprises introducing into a plant cell an exogenous nucleic acid comprising a nucleotide sequence encoding a polypeptide, where the HMM bit score of the amino acid sequence of the polypeptide is greater than 50, the HMM based on the amino acid sequences depicted in one of FIGS. 1-18, and where a tissue of a plant produced from the plant cell has a difference in the level of protein as compared to the corresponding level in tissue of a control plant that does not comprise the exogenous nucleic acid.

In another aspect, a method of modulating the level of protein in a plant is provided. The method comprises introducing into a plant cell an exogenous nucleic acid comprising a nucleotide sequence encoding a polypeptide 208-257 amino acids in length, where the polypeptide is the amino terminus of a polypeptide having at least 500 amino acids and having an HMM bit score greater than 712, the HMM based on the amino acid sequences depicted in FIG. 15, and where a tissue of a plant produced from the plant cell has a difference in the level of protein as compared to the corresponding level in tissue of a control plant that does not comprise the exogenous nucleic acid.

In another aspect, a method of modulating the level of protein in a plant is provided. The method comprises introducing into a plant cell an exogenous nucleic acid comprising a nucleotide sequence encoding a polypeptide 330-430 amino acids in length, where the polypeptide is the carboxy terminus of a polypeptide having at least 500 amino acids and having an HMM bit score greater than 724, the HMM based on the amino acid sequences depicted in FIG. 17, and where a tissue of a plant produced from the plant cell has a difference in the level of protein as compared to the corresponding level in tissue of a control plant that does not comprise the exogenous nucleic acid.

In another aspect, a method of modulating the level of protein in a plant is provided. The method comprises introducing into a plant cell an exogenous nucleic acid comprising a nucleotide sequence encoding a polypeptide having 80 percent or greater sequence identity to an amino acid sequence selected from the group consisting of SEQ ID NOs:80-82, SEQ ID NOs:84-93, SEQ ID NOs:95-96, SEQ ID NOs:98-100, SEQ ID NOs:102-103, SEQ ID NO:105, SEQ ID NO:107, SEQ ID NOs:109-110, SEQ ID NO:112, SEQ ID NO:114, SEQ ID NOs:116-117, SEQ ID NOs:119-122, SEQ ID NOs:124-128, SEQ ID NO:130, SEQ ID NOs:132-133, SEQ ID NOs:135-139, SEQ ID NOs:141-150, SEQ ID NO:152, SEQ ID NOs:154-155, SEQ ID NOs:157-159, SEQ ID NOs:161-162, SEQ ID NO:164, SEQ ID NOs:166-169, SEQ ID NO:171, SEQ ID NO:173, SEQ ID NOs:175-178, SEQ ID NO:180, SEQ ID NOs:182-187, SEQ ID NO:189, SEQ ID NOs:191-196, SEQ ID NOs:198-203, SEQ ID NO:205, SEQ ID NO:209, SEQ ID NOs:211-212, SEQ ID NOs:214-215, SEQ ID NOs:217-218, SEQ ID NO:220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NOs:248-250, SEQ ID NO:252, SEQ ID NO:254, SEQ ID NOs:256-286, SEQ ID NO:315, SEQ ID NOs:317-328, SEQ ID NOs:330, SEQ ID NO:332, SEQ ID NO:334, SEQ ID NOs:336-337, SEQ ID NO:339, SEQ ID NO:341, and SEQ ID NOs:343-349, where a tissue of a plant produced from the plant cell has a difference in the level of protein as compared to the corresponding level in tissue of a control plant that does not comprise the exogenous nucleic acid. The nucleotide sequence can encode a polypeptide comprising an amino acid sequence corresponding to SEQ ID NO:130.

In another aspect, a method of modulating the level of protein in a plant is provided. The method comprises introducing into a plant cell an exogenous nucleic acid comprising a nucleotide sequence having 80 percent or greater sequence identity to a nucleotide sequence selected from the group consisting of SEQ ID NO:79, SEQ ID NO:83, SEQ ID NO:94, SEQ ID NO:97, SEQ ID NO:101, SEQ ID NO:104, SEQ ID NO:106, SEQ ID NO:108, SEQ ID NO:111, SEQ ID NO:113, SEQ ID NO:115, SEQ ID NO:118, SEQ ID NO:123, SEQ ID NO:129, SEQ ID NO:131, SEQ ID NO:134, SEQ ID NO:140, SEQ ID NO:151, SEQ ID NO:153, SEQ ID NO:156, SEQ ID NO:160, SEQ ID NO:163, SEQ ID NO:165, SEQ ID NO:170, SEQ ID NO:172, SEQ ID NO:174, SEQ ID NO:179, SEQ ID NO:181, SEQ ID NO:188, SEQ ID NO:190, SEQ ID NO:197, SEQ ID NO:204, SEQ ID NO:206, SEQ ID NO:208, SEQ ID NO:210, SEQ ID NO:213, SEQ ID NO:216, SEQ ID NO:219, SEQ ID NO:221, SEQ ID NO:223, SEQ ID NO:225, SEQ ID NO:227, SEQ ID NO:229, SEQ ID NO:231, SEQ ID NO:233, SEQ ID NO:235, SEQ ID NO:237, SEQ ID NO:239, SEQ ID NO:241, SEQ ID NO:243, SEQ ID NO:245, SEQ ID NO:247, SEQ ID NO:251, SEQ ID NO:253, SEQ ID NO:255, SEQ ID NOs:287-314, SEQ ID NO:316, SEQ ID NO:329, SEQ ID NO:331, SEQ ID NO:333, SEQ ID NO:335, SEQ ID NO:338, SEQ ID NO:340, and SEQ ID NO:342, where a tissue of a plant produced from the plant cell has a difference in the level of protein as compared to the corresponding level in tissue of a control plant that does not comprise the exogenous nucleic acid. The nucleotide sequence can comprise the nucleotide sequence set forth in SEQ ID NO:206.

The difference can be an increase in the level of protein. The exogenous nucleic acid can be operably linked to a regulatory region.

In another aspect, a method of modulating the level of protein in a plant is provided. The method comprises introducing into a plant cell an exogenous nucleic acid comprising a regulatory region operably linked to a polynucleotide whose transcription product is at least 30 nucleotides in length and is complementary to a nucleic acid encoding a polypeptide, where the HMM bit score of the amino acid sequence of the polypeptide is greater than 50, the HMM based on the amino acid sequences depicted in one of FIGS. 1-18, where the regulatory region modulates transcription of the polynucleotide in the plant cell, and where a tissue of a plant produced from the plant cell has a difference in the level of protein as compared to the corresponding level in tissue of a control plant that does not comprise the exogenous nucleic acid. The HMM bit score can be 100 or greater.

In another aspect, a method of modulating the level of protein in a plant is provided. The method comprises introducing into a plant cell an exogenous nucleic acid comprising a regulatory region operably linked to a polynucleotide that is transcribed into an interfering RNA effective for inhibiting expression of a polypeptide having 80 percent or greater sequence identity to an amino acid sequence selected from the group consisting of SEQ ID NOs:80-82, SEQ ID NOs:84-93, SEQ ID NOs:95-96, SEQ ID NOs:98-100, SEQ ID NOs:102-103, SEQ ID NO:105, SEQ ID NO:107, SEQ ID NOs:109-110, SEQ ID NO:112, SEQ ID NO:114, SEQ ID NOs:116-117, SEQ ID NOs:119-122, SEQ ID NOs:124-128, SEQ ID NO:130, SEQ ID NOs:132-133, SEQ ID NOs:135-139, SEQ ID NOs:141-150, SEQ ID NO:152, SEQ ID NOs:154-155, SEQ ID NOs:157-159, SEQ ID NOs:161-162, SEQ ID NO:164, SEQ ID NOs:166-169, SEQ ID NO:171, SEQ ID NO:173, SEQ ID NOs:175-178, SEQ ID NO:180, SEQ ID NOs:182-187, SEQ ID NO:189, SEQ ID NOs:191-196, SEQ ID NOs:198-203, SEQ ID NO:205, SEQ ID NO:209, SEQ ID NOs:211-212, SEQ ID NOs:214-215, SEQ ID NOs:217-218, SEQ ID NO:220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NOs:248-250, SEQ ID NO:252, SEQ ID NO:254, SEQ ID NOs:256-286, SEQ ID NO:315, SEQ ID NOs:317-328, SEQ ID NOs:330, SEQ ID NO:332, SEQ ID NO:334, SEQ ID NOs:336-337, SEQ ID NO:339, SEQ ID NO:341, and SEQ ID NOs:343-349, where the regulatory region modulates transcription of the polynucleotide in the plant cell, and where a tissue of a plant produced from the plant cell has a difference in the level of protein as compared to the corresponding level in tissue of a control plant that does not comprise the exogenous nucleic acid. The exogenous nucleic acid can further comprise a 3′ UTR operably linked to the polynucleotide. The polynucleotide can be transcribed into an interfering RNA comprising a stem-loop structure. The stem-loop structure: can comprise an inverted repeat of the 3′ UTR.

The difference can be a decrease in the level of protein. The sequence identity can be 85 percent or greater, 90 percent or greater, or 95 percent or greater. The method can further comprise the step of producing a plant from the plant cell. The introducing step can comprise introducing the nucleic acid into a plurality of plant cells. The method can further comprise the step of producing a plurality of plants from the plant cells. The method can further comprise the step of selecting one or more plants from the plurality of plants that have the difference in the level of protein. The regulatory region can be a tissue-preferential, broadly expressing, or inducible promoter.

In another aspect, a method of producing a plant tissue is provided. The method comprises growing a plant cell comprising an exogenous nucleic acid comprising a nucleotide sequence encoding a polypeptide, where the HMM bit score of the amino acid sequence of the polypeptide is greater than 50, the HMM based on the amino acid sequences depicted in one of FIGS. 1-18, and where the tissue has a difference in the level of protein as compared to the corresponding level in tissue of a control plant that does not comprise the exogenous nucleic acid.

In another aspect, a method of producing a plant tissue is provided. The method comprises growing a plant cell comprising an exogenous nucleic acid comprising a nucleotide sequence encoding a polypeptide 208-257 amino acids in length, where the polypeptide is the amino terminus of a polypeptide having at least 500 amino acids and having an HMM bit score greater than 712, the HMM based on the amino acid sequences depicted in FIG. 15, and where the tissue has a difference in the level of protein as compared to the corresponding level in tissue of a control plant that does not comprise the nucleic acid.

In another aspect, a method of producing a plant tissue is provided. The method comprises growing a plant cell comprising an exogenous nucleic acid comprising a nucleotide sequence encoding a polypeptide 330-430 amino acids in length, where the polypeptide is the carboxy terminus of a polypeptide having at least 500 amino acids and having an HMM bit score greater than 724, the HMM based on the amino acid sequences depicted in FIG. 17, and where the tissue has a difference in the level of protein as compared to the corresponding level in tissue of a control plant that does not comprise the nucleic acid.

In another aspect, a method of producing a plant tissue is provided. The method comprises growing a plant cell comprising an exogenous nucleic acid comprising a nucleotide sequence encoding a polypeptide having 80 percent or greater sequence identity to an amino acid sequence selected from the group consisting of SEQ ID NOs:80-82, SEQ ID NOs:84-93, SEQ ID NOs:95-96, SEQ ID NOs:98-100, SEQ ID NOs:102-103, SEQ ID NO:105, SEQ ID NO:107, SEQ ID NOs:109-110, SEQ ID NO:112, SEQ ID NO:114, SEQ ID NOs:116-117, SEQ ID NOs:119-122, SEQ ID NOs:124-128, SEQ ID NO:130, SEQ ID NOs:132-133, SEQ ID NOs:135-139, SEQ ID NOs:141-150, SEQ ID NO:152, SEQ ID NOs:154-155, SEQ ID NOs:157-159, SEQ ID NOs:161-162, SEQ ID NO:164, SEQ ID NOs:166-169, SEQ ID NO:171, SEQ ID NO:173, SEQ ID NOs:175-178, SEQ ID NO:180, SEQ ID NOs:182-187, SEQ ID NO:189, SEQ ID NOs:191-196, SEQ ID NOs:198-203, SEQ ID NO:205, SEQ ID NO:209, SEQ ID NOs:211-212, SEQ ID NOs:214-215, SEQ ID NOs:217-218, SEQ ID NO:220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NOs:248-250, SEQ ID NO:252, SEQ ID NO:254, SEQ ID NOs:256-286, SEQ ID NO:315, SEQ ID NOs:317-328, SEQ ID NOs:330, SEQ ID NO:332, SEQ ID NO:334, SEQ ID NOs:336-337, SEQ ID NO:339, SEQ ID NO:341, and SEQ ID NOs:343-349, where the tissue has a difference in the level of protein as compared to the corresponding level in tissue of a control plant that does not comprise the nucleic acid.

In another aspect, a method of producing a plant tissue is provided. The method comprises growing a plant cell comprising an exogenous nucleic acid comprising a nucleotide sequence having 80 percent or greater sequence identity to a nucleotide sequence selected from the group consisting of SEQ ID NO:79, SEQ ID NO:83, SEQ ID NO:94, SEQ ID NO:97, SEQ ID NO:101, SEQ ID NO:104, SEQ ID NO:106, SEQ ID NO:108, SEQ ID NO:111, SEQ ID NO:113, SEQ ID NO:115, SEQ ID NO:118, SEQ ID NO:123, SEQ ID NO:129, SEQ ID NO:131, SEQ ID NO:134, SEQ ID NO:140, SEQ ID NO:151, SEQ ID NO:153, SEQ ID NO:156, SEQ ID NO:160, SEQ ID NO:163, SEQ ID NO:165, SEQ ID NO:170, SEQ ID NO:172, SEQ ID NO:174, SEQ ID NO:179, SEQ ID NO:181, SEQ ID NO:188, SEQ ID NO:190, SEQ ID NO:197, SEQ ID NO:204, SEQ ID NO:206, SEQ ID NO:208, SEQ ID NO:210, SEQ ID NO:213, SEQ ID NO:216, SEQ ID NO:219, SEQ ID NO:221, SEQ ID NO:223, SEQ ID NO:225, SEQ ID NO:227, SEQ ID NO:229, SEQ ID NO:231, SEQ ID NO:233, SEQ ID NO:235, SEQ ID NO:237, SEQ ID NO:239, SEQ ID NO:241, SEQ ID NO:243, SEQ ID NO:245, SEQ ID NO:247, SEQ ID NO:251, SEQ ID NO:253, SEQ ID NO:255, SEQ ID NOs:287-314, SEQ ID NO:316, SEQ ID NO:329, SEQ ID NO:331, SEQ ID NO:333, SEQ ID NO:335, SEQ ID NO:338, SEQ ID NO:340, and SEQ ID NO:342, where the tissue has a difference in the level of protein as compared to the corresponding level in tissue of a control plant that does not comprise the nucleic acid.

In another aspect, a method of producing a plant tissue is provided. The method comprises growing a plant cell comprising an exogenous nucleic acid comprising a regulatory region operably linked to a polynucleotide whose transcription product is at least 30 nucleotides in length and is complementary to a nucleic acid encoding a polypeptide, where the HMM bit score of the amino acid sequence of the polypeptide is greater than 50, the HMM based on the amino acid sequences depicted in one of FIGS. 1-18, where the regulatory region modulates transcription of the polynucleotide in the plant cell, and where the tissue has a difference in the level of protein as compared to the corresponding level in tissue of a control plant that does not comprise the nucleic acid.

In another aspect, a method of producing a plant tissue is provided. The method comprises growing a plant cell comprising an exogenous nucleic acid comprising a regulatory region operably linked to a polynucleotide that is transcribed into an interfering RNA effective for inhibiting expression of a polypeptide having 80 percent or greater sequence identity to an amino acid sequence selected from the group consisting of SEQ ID NOs:80-82, SEQ ID NOs:84-93, SEQ ID NOs:95-96, SEQ ID NOs:98-100, SEQ ID NOs:102-103, SEQ ID NO:105, SEQ ID NO:107, SEQ ID NOs:109-110, SEQ ID NO:112, SEQ ID NO:114, SEQ ID NOs:116-117, SEQ ID NOs:119-122, SEQ ID NOs:124-128, SEQ ID NO:130, SEQ ID NOs:132-133, SEQ ID NOs:135-139, SEQ ID NOs:141-150, SEQ ID NO:152, SEQ ID NOs:154-155, SEQ ID NOs:157-159, SEQ ID NOs:161-162, SEQ ID NO:164, SEQ ID NOs:166-169, SEQ ID NO:171, SEQ ID NO:173, SEQ ID NOs:175-178, SEQ ID NO:180, SEQ ID NOs:182-187, SEQ ID NO:189, SEQ ID NOs:191-196, SEQ ID NOs:198-203, SEQ ID NO:205, SEQ ID NO:209, SEQ ID NOs:211-212, SEQ ID NOs:214-215, SEQ ID NOs:217-218, SEQ ID NO:220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NOs:248-250, SEQ ID NO:252, SEQ ID NO:254, SEQ ID NOs:256-286, SEQ ID NO:315, SEQ ID NOs:317-328, SEQ ID NOs:330, SEQ ID NO:332, SEQ ID NO:334, SEQ ID NOs:336-337, SEQ ID NO:339, SEQ ID NO:341, and SEQ ID NOs:343-349, where the regulatory region modulates transcription of the polynucleotide in the plant cell, and where the tissue has a difference in the level of protein as compared to the corresponding level in tissue of a control plant that does not comprise the nucleic acid.

The plant can be a dicot. The plant can be a species selected from the group consisting of Beta vulgaris (sugarbeet), Brassica napus (canola), Glycine max (soybean), Helianthus annuus (sunflower), Lupinus albus (lupin), or Medicago sativa (alfalfa). The plant can be a monocot. The plant can be a species selected from the group consisting of Oryza sativa (rice), Pennisetum glaucum (pearl millet), Triticum aestivum, (wheat), or Zea mays (corn). The tissue can be seed tissue.

In another aspect, a plant cell is provided. The plant cell comprises an exogenous nucleic acid comprising a nucleotide sequence encoding a polypeptide, where the HMM bit score of the amino acid sequence of the polypeptide is greater than 50, the HMM based on the amino acid sequences depicted in one of FIGS. 1-18, and where a tissue of a plant produced from the plant cell has a difference in the level of protein as compared to the corresponding level in tissue of a control plant that does not comprise the nucleic acid.

In another aspect, a plant cell is provided. The plant cell comprises an exogenous nucleic acid comprising a nucleotide sequence encoding a polypeptide 208-257 amino acids in length, where the polypeptide is the amino terminus of a polypeptide having at least 500 amino acids and having an HMM bit score greater than 712, the HMM based on the amino acid sequences depicted in FIG. 15, and where a tissue of a plant produced from the plant cell has a difference in the level of protein as compared to the corresponding level in tissue of a control plant that does not comprise the nucleic acid.

In another aspect, a plant cell is provided. The plant cell comprises an exogenous nucleic acid comprising a nucleotide sequence encoding a polypeptide 330-430 amino acids in length, where the polypeptide is the carboxy terminus of a polypeptide having at least 500 amino acids and having an HMM bit score greater than 724, the HMM based on the amino acid sequences depicted in FIG. 17, and where a tissue of a plant produced from the plant cell has a difference in the level of protein as compared to the corresponding level in tissue of a control plant that does not comprise the nucleic acid.

In another aspect, a plant cell is provided. The plant cell comprises an exogenous nucleic acid comprising a nucleotide sequence encoding a polypeptide having 80 percent or greater sequence identity to an amino acid sequence selected from the group consisting of SEQ ID NOs:80-82, SEQ ID NOs:84-93, SEQ ID NOs:95-96, SEQ ID NOs:98-100, SEQ ID NOs:102-103, SEQ ID NO:105, SEQ ID NO:107, SEQ ID NOs:109-110, SEQ ID NO:112, SEQ ID NO:114, SEQ ID NOs:116-117, SEQ ID NOs:119-122, SEQ ID NOs:124-128, SEQ ID NO:130, SEQ ID NOs:132-133, SEQ ID NOs:135-139, SEQ ID NOs:141-150, SEQ ID NO:152, SEQ ID NOs:154-155, SEQ ID NOs:157-159, SEQ ID NOs:161-162, SEQ ID NO:164, SEQ ID NOs:166-169, SEQ ID NO:171, SEQ ID NO:173, SEQ ID NOs:175-178, SEQ ID NO:180, SEQ ID NOs:182-187, SEQ ID NO:189, SEQ ID NOs:191-196, SEQ ID NOs:198-203, SEQ ID NO:205, SEQ ID NO:209, SEQ ID NOs:211-212, SEQ ID NOs:214-215, SEQ ID NOs:217-218, SEQ ID NO:220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NOs:248-250, SEQ ID NO:252, SEQ ID NO:254, SEQ ID NOs:256-286, SEQ ID NO:315, SEQ ID NOs:317-328, SEQ ID NOs:330, SEQ ID NO:332, SEQ ID NO:334, SEQ ID NOs:336-337, SEQ ID NO:339, SEQ ID NO:341, and SEQ ID NOs:343-349, where a tissue of a plant produced from the plant cell has a difference in the level of protein as compared to the corresponding level in tissue of a control plant that does not comprise the nucleic acid.

In another aspect, a plant cell is provided. The plant cell comprises an exogenous nucleic acid comprising a nucleotide sequence having 80 percent or greater sequence identity to a nucleotide sequence selected from the group consisting of SEQ ID NO:79, SEQ ID NO:83, SEQ ID NO:94, SEQ ID NO:97, SEQ ID NO:101, SEQ ID NO:104, SEQ ID NO:106, SEQ ID NO:108, SEQ ID NO:111, SEQ ID NO:113, SEQ ID NO:115, SEQ ID NO:118, SEQ ID NO:123, SEQ ID NO:129, SEQ ID NO:131, SEQ ID NO:134, SEQ ID NO:140, SEQ ID NO:151, SEQ ID NO:153, SEQ ID NO:156, SEQ ID NO:160, SEQ ID NO:163, SEQ ID NO:165, SEQ ID NO:170, SEQ ID NO:172, SEQ ID NO:174, SEQ ID NO:179, SEQ ID NO:181, SEQ ID NO:188, SEQ ID NO:190, SEQ ID NO:197, SEQ ID NO:204, SEQ ID NO:206, SEQ ID NO:208, SEQ ID NO:210, SEQ ID NO:213, SEQ ID NO:216, SEQ ID NO:219, SEQ ID NO:221, SEQ ID NO:223, SEQ ID NO:225, SEQ ID NO:227, SEQ ID NO:229, SEQ ID NO:231, SEQ ID NO:233, SEQ ID NO:235, SEQ ID NO:237, SEQ ID NO:239, SEQ ID NO:241, SEQ ID NO:243, SEQ ID NO:245, SEQ ID NO:247, SEQ ID NO:251, SEQ ID NO:253, SEQ ID NO:255, SEQ ID NOs:287-314, SEQ ID NO:316, SEQ ID NO:329, SEQ ID NO:331, SEQ ID NO:333, SEQ ID NO:335, SEQ ID NO:338, SEQ ID NO:340, and SEQ ID NO:342, where a tissue of a plant produced from the plant cell has a difference in the level of protein as compared to the corresponding level in tissue of a control plant that does not comprise the nucleic acid.

In another aspect, a plant cell is provided. The plant cell comprises an exogenous nucleic acid comprising a regulatory region operably linked to a polynucleotide whose transcription product is at least 30 nucleotides in length and is complementary to a nucleic acid encoding a polypeptide, where the HMM bit score of the amino acid sequence of the polypeptide is greater than 50, the HMM based on the amino acid sequences depicted in one of FIGS. 1-18, where the regulatory region modulates transcription of the polynucleotide in the plant cell, and where a tissue of a plant produced from the plant cell has a difference in the level of protein as compared to the corresponding level in tissue of a control plant that does not comprise the nucleic acid.

In another aspect, a plant cell is provided. The plant cell comprises an exogenous nucleic acid comprising a regulatory region operably linked to a polynucleotide that is transcribed into an interfering RNA effective for inhibiting expression of a polypeptide having 80 percent or greater sequence identity to an amino acid sequence selected from the group consisting of SEQ ID NOs:80-82, SEQ ID NOs:84-93, SEQ ID NOs:95-96, SEQ ID NOs:98-100, SEQ ID NOs:102-103, SEQ ID NO:105, SEQ ID NO:107, SEQ ID NOs:109-110, SEQ ID NO:112, SEQ ID NO:114, SEQ ID NOs:116-117, SEQ ID NOs:119-122, SEQ ID NOs:124-128, SEQ ID NO:130, SEQ ID NOs:132-133, SEQ ID NOs:135-139, SEQ ID NOs:141-150, SEQ ID NO:152, SEQ ID NOs:154-155, SEQ ID NOs:157-159, SEQ ID NOs:161-162, SEQ ID NO:164, SEQ ID NOs:166-169, SEQ ID NO:171, SEQ ID NO:173, SEQ ID NOs:175-178, SEQ ID NO:180, SEQ ID NOs:182-187, SEQ ID NO:189, SEQ ID NOs:191-196, SEQ ID NOs:198-203, SEQ ID NO:205, SEQ ID NO:209, SEQ ID NOs:211-212, SEQ ID NOs:214-215, SEQ ID NOs:217-218, SEQ ID NO:220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NOs:248-250, SEQ ID NO:252, SEQ ID NO:254, SEQ ID NOs:256-286, SEQ ID NO:315, SEQ ID NOs:317-328, SEQ ID NOs:330, SEQ ID NO:332, SEQ ID NO:334, SEQ ID NOs:336-337, SEQ ID NO:339, SEQ ID NO:341, and SEQ ID NOs:343-349, where the regulatory region modulates transcription of the polynucleotide in the plant cell, and where a tissue of a plant produced from the plant cell has a difference in the level of protein as compared to the corresponding level in tissue of a control plant that does not comprise the nucleic acid.

The plant can be a dicot. The plant can be a species selected from the group consisting of Beta vulgaris (sugarbeet), Brassica napus (canola), Glycine max (soybean), Helianthus annuus (sunflower), Lupinus albus (lupin), or Medicago saliva (alfalfa). The plant can be a monocot. The plant can be a species selected from the group consisting of Oryza saliva (rice), Pennisetum glaucum (pearl millet), Triticum aestivum, (wheat), or Zea mays (corn). The tissue can be seed tissue.

A transgenic plant is also provided. The transgenic plant comprises any of the plant cells described above. Progeny of the plant are also provided. The progeny has a difference in the level of protein as compared to the level of protein in a corresponding control plant that does not comprise the exogenous nucleic acid. Seed, vegetative tissue, and fruit from the transgenic plant are also provided.

In another aspect, an isolated nucleic acid is provided. The isolated nucleic acid comprises a nucleotide sequence having 95% or greater sequence identity to a nucleotide sequence selected from the group consisting of SEQ ID NO:79, SEQ ID NO:83, SEQ ID NO:94, SEQ ID NO:97, SEQ ID NO:101, SEQ ID NO:104, SEQ ID NO:106, SEQ ID NO:108, SEQ ID NO:11, SEQ ID NO:113, SEQ ID NO:115, SEQ ID NO:118, SEQ ID NO:123, SEQ ID NO:129, SEQ ID NO:131, SEQ ID NO:134, SEQ ID NO:140, SEQ ID NO:151, SEQ ID NO:153, SEQ ID NO:156, SEQ ID NO:160, SEQ ID NO:163, SEQ ID NO:165, SEQ ID NO:170, SEQ ID NO:172, SEQ ID NO:174, SEQ ID NO:179, SEQ ID NO:181, SEQ ID NO:188, SEQ ID NO:190, SEQ ID NO:197, SEQ ID NO:204, SEQ ID NO:206, SEQ ID NO:208, SEQ ID NO:210, SEQ ID NO:213, SEQ ID NO:216, SEQ ID NO:219, SEQ ID NO:221, SEQ ID NO:223, SEQ ID NO:225, SEQ ID NO:227, SEQ ID NO:229, SEQ ID NO:231, SEQ ID NO:233, SEQ ID NO:235, SEQ ID NO:237, SEQ ID NO:239, SEQ ID NO:241, SEQ ID NO:243, SEQ ID NO:245, SEQ ID NO:247, SEQ ID NO:251, SEQ ID NO:253, SEQ ID NO:255, SEQ ID NOs:287-314, SEQ ID NO:316, SEQ ID NO:329, SEQ ID NO:331, SEQ ID NO:333, SEQ ID NO:335, SEQ ID NO:338, SEQ ID NO:340, and SEQ ID NO:342.

In another aspect, an isolated nucleic acid is provided. The isolated nucleic acid comprises a nucleotide sequence encoding a polypeptide having 80% or greater sequence identity to an amino acid sequence selected from the group consisting of SEQ ID NOs:80-82, SEQ ID NO:84, SEQ ID NO:89, SEQ ID NO:95, SEQ ID NO:98, SEQ ID NO:100, SEQ ID NOs:102-103, SEQ ID NO:105, SEQ ID NO:107, SEQ ID NOs:109-110, SEQ ID NO:112, SEQ ID NO:114, SEQ ID NOs:116-117, SEQ ID NOs:119-120, SEQ ID NO:122, SEQ ID NOs:124-127, SEQ ID NO:130, SEQ ID NOs:132-133, SEQ ID NOs:135-136, SEQ ID NOs:138-139, SEQ ID NO:141, SEQ ID NO:149, SEQ ID NO:152, SEQ ID NO:154, SEQ ID NOs:157-158, SEQ ID NO:161, SEQ ID NO:164, SEQ ID NOs:166-167, SEQ ID NO:171, SEQ ID NO:173, SEQ ID NOs:175-178, SEQ ID NO:180, SEQ ID NOs:182-185, SEQ ID NO:187, SEQ ID NO:189, SEQ ID NO:191, SEQ ID NO:193, SEQ ID NO:198, SEQ ID NO:205, SEQ ID NO:209, SEQ ID NOs:211-212, SEQ ID NOs:214-215, SEQ ID NOs:217-218, SEQ ID NO:220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NOs:248-250, SEQ ID NO:252, SEQ ID NO:254, SEQ ID NO:256, SEQ ID NO:315, SEQ ID NO:317, SEQ ID NOs:322, SEQ ID NOs:325-326, SEQ ID NO:330, SEQ ID NO:332, SEQ ID NO:334, SEQ ID NOs:336-337, SEQ ID NO:339, SEQ ID NO:341, SEQ ID NO:343, and SEQ ID NOs:346-349.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is an alignment of CLONE 33780 (SEQ ID NO:80) with homologous and/or orthologous amino acid sequences CLONE 1082418 (SEQ ID NO:81), CLONE 1058516 (SEQ ID NO:82), and CLONE 1808721 (SEQ ID NO:224). FIG. 1 and the other alignment figures provided herein were generated using the program MUSCLE version 3.52 (Edgar, Nucleic Acids Res, 32(5):1792-97 (2004); World Wide Web at drive5.com/muscle).

FIG. 2 is an alignment of cDNA 7089429 (SEQ ID NO:84) with homologous and/or orthologous amino acid sequences GI 58201026 (SEQ ID NO:92), GI 14422402 (SEQ ID NO:93), ANNOT 1457156 (SEQ ID NO:214), CLONE 1811354 (SEQ ID NO:226), CLONE 1894727 (SEQ ID NO:240), CLONE 470181 (SEQ ID NO:248), CLONE 753701 (SEQ ID NO:254), GI 115473007 (SEQ ID NO:257), GI 116060748 (SEQ ID NO:258), GI 121145 (SEQ ID NO:259), GI 13431546 (SEQ ID NO:261), GI 13431547 (SEQ ID NO:262), GI 17352451 (SEQ ID NO:263), GI 18146809 (SEQ ID NO:264), GI 20386368 (SEQ ID NO:265), GI 34484306 (SEQ ID NO:267), GI 3885426 (SEQ ID NO:268), GI 41059107 (SEQ ID NO:269), GI 4322331 (SEQ ID NO:270), GI 462-41274 (SEQ ID NO:271), GI 4958918 (SEQ ID NO:272), GI 56122554 (SEQ ID NO:273), GI 6277254 (SEQ ID NO:274), GI 6277256 (SEQ ID NO:275), GI 6449052 (SEQ ID NO:276), GI 75250205 (SEQ ID NO:277), GI 82547882 (SEQ ID NO:279), GI 87299435 (SEQ ID NO:280), GI 88910043 (SEQ ID NO:281), GI 90289577 (SEQ ID NO:282), GI 92868507 (SEQ ID NO:284), and GI 9971808 (SEQ ID NO:286).

FIG. 3 is an alignment of CLONE 285705 (SEQ ID NO:95) with homologous and/or orthologous amino acid sequences GI 50918655 (SEQ ID NO:96), ANNOT 1505632 (SEQ ID NO:98), GI 16323464 (SEQ ID NO:99), and CLONE 1812252 (SEQ ID NO:228).

FIG. 4 is an alignment of CLONE 42577 (SEQ ID NO:102) with homologous and/or orthologous amino acid sequences CLONE 1439269 (SEQ ID NO:103), ANNOT 1493706 (SEQ ID NO:107), CLONE 645909 (SEQ ID NO:10), and CLONE 1834121 (SEQ ID NO:230).

FIG. 5 is an alignment of ANNOT 840247 (SEQ ID NO:114) with homologous and/or orthologous amino acid sequences ANNOT 1453934 (SEQ ID NO:116) and CLONE 512894 (SEQ ID NO:117).

FIG. 6 is an alignment of CLONE 400568 (SEQ ID NO:119) with homologous and/or orthologous amino acid sequences GI 37718893 (SEQ ID NO:121), CLONE 937503 (SEQ ID NO:122), ANNOT 1503141 (SEQ ID NO:124), CLONE 625275 (SEQ ID NO:125), GI 111994767 (SEQ ID NO:128), CLONE 1719600 (SEQ ID NO:220), and CLONE 1838546 (SEQ ID NO:232).

FIG. 7 is an alignment of ANNOT 574310 (SEQ ID NO:130) with homologous and/or orthologous amino acid sequences ANNOT 1522260 (SEQ ID NO:132), CLONE 625135 (SEQ ID NO:133), GI 50927857 (SEQ ID NO:137), CLONE 843076 (SEQ ID NO:138), CLONE 296774 (SEQ ID NO:139), and CLONE 1999828 (SEQ ID NO:244).

FIG. 8 is an alignment of CLONE 1103471 (SEQ ID NO:141) with homologous and/or orthologous amino acid sequences GI 21618143 (SEQ ID NO:142), GI 4666360 (SEQ ID NO:144), GI 33771374 (SEQ ID NO:145), GI 439493 (SEQ ID NO:146), GI 71979887 (SEQ ID NO:147), GI 33331578 (SEQ ID NO:148), CLONE 1240096 (SEQ ID NO:149), GI 7228329 (SEQ ID NO:150), ANNOT 1496702 (SEQ ID NO:152), GI 32441471 (SEQ ID NO:155), ANNOT 1470888 (SEQ ID NO:157), and GI 55734108 (SEQ ID NO:159).

FIG. 9 is an alignment of ANNOT 543117 (SEQ ID NO:161) with homologous and/or orthologous amino acid sequences ANNOT 1464138 (SEQ ID NO:164), CLONE 481263 (SEQ ID NO:167), GI 50929499 (SEQ ID NO:168), CLONE 1806767 (SEQ ID NO:222), CLONE 378258 (SEQ ID NO:246), GI 90657540 (SEQ ID NO:283), and GI 92894700 (SEQ ID NO:285).

FIG. 10 is an alignment of ANNOT 546661 (SEQ ID NO:171) with homologous and/or orthologous amino acid sequence ANNOT 1467926 (SEQ ID NO:173).

FIG. 11 is an alignment of ANNOT 570373 (SEQ ID NO:175) with homologous and/or orthologous amino acid sequence CLONE 1607448 (SEQ ID NO:176).

FIG. 12 is an alignment of CLONE 531679 (SEQ ID NO:182) with homologous and/or orthologous amino acid sequences CLONE 1054809 (SEQ ID NO:185), G178191452 (SEQ ID NO:186), CLONE 244926 (SEQ ID NO:187), ANNOT 1586846 (SEQ ID NO:189), CLONE 1841382 (SEQ ID NO:236), and GI 125563536 (SEQ ID NO:260).

FIG. 13 is an alignment of CLONE 558363 (SEQ ID NO:191) with homologous and/or orthologous amino acid sequences GI 3413322 (SEQ ID NO:192), GI 41529571 (SEQ ID NO:194), ANNOT 1540806 (SEQ ID NO:198), GI 6714530 (SEQ ID NO:199), and GI 27902548 (SEQ ID NO:200).

FIG. 14 is an alignment of ANNOT 830572 (SEQ ID NO:209) with homologous and/or orthologous amino acid sequences ANNOT 1497025 (SEQ ID NO:211) and CLONE 1659056 (SEQ ID NO:212).

FIG. 15 is an alignment of LOCUS AT2G35155 (SEQ ID NO:349) with homologous and/or orthologous amino acid sequences ANNOT 1527550 (SEQ ID NO:315), GI 38344253 (SEQ ID NO:318), and GI 124359654 (SEQ ID NO:320).

FIG. 16 is an alignment of LOCUS AT2G35155·T (SEQ ID NO:348) with homologous and/or orthologous amino acid sequences GI 125561508·T (SEQ ID NO:323), ANNOT 1527550·T (SEQ ID NO:325), and GI 124359654·T (SEQ ID NO:327).

FIG. 17 is an alignment of LOCUS ATIG78230 (SEQ ID NO:337) with homologous and/or orthologous amino acid sequences ANNOT 1451858 (SEQ ID NO:330), CLONE 1574720 (SEQ ID NO:332), CLONE 1862739 (SEQ ID NO:334), CLONE 546776 (SEQ ID NO:336), CLONE 1928737 (SEQ ID NO:343), and GI 115481758 (SEQ ID NO:344).

FIG. 18 is an alignment of LOCUS AT1G78230·T (SEQ ID NO:256) with homologous and/or orthologous amino acid sequences ANNOT 1451858·T (SEQ ID NO:346), CLONE 1574720·T (SEQ ID NO:347), CLONE 1928737·T (SEQ ID NO:86), GI 1115481758·T (SEQ ID NO:183), CLONE 1813489·T (SEQ ID NO:249), and CLONE 546776·T (SEQ ID NO:252).

### DETAILED DESCRIPTION

The invention features methods and materials related to modulating (e.g., increasing or decreasing) protein levels in plants. In some embodiments, the plants may also have modulated levels of oil. The methods can include transforming a plant cell with a nucleic acid encoding a protein-modulating polypeptide, wherein expression of the polypeptide results in a modulated level of protein. Plant cells produced using such methods can be grown to produce plants having an increased or decreased protein content. Such plants, and the seeds of such plants, may be used to produce, for example, foodstuffs and animal, feed having an increased protein content and nutritional value.

### Polypeptides

The term “polypeptide” as used herein refers to a compound of two or more subunit amino acids, amino acid analogs, or other peptidomimetics, regardless of post-translational modification, e.g., phosphorylation or glycosylation. The subunits may be linked by peptide bonds or other bonds such as, for example, ester or ether bonds. The term “amino acid” refers to natural and/or unnatural or synthetic amino acids, including D/L optical isomers. Full-length proteins, analogs, mutants, and fragments thereof are encompassed by this definition.

Polypeptides described herein include protein-modulating polypeptides. Protein-modulating polypeptides can be effective to modulate protein levels when expressed in a plant or plant cell. Modulation of the level of protein can be either an increase or a decrease in the level of protein relative to the corresponding level in control plants.

A protein-modulating polypeptide can contain a polyprenyl_synt domain characteristic of a polyprenyl synthetase polypeptide, such as a geranylgeranyl pyrophosphate synthase polypeptide. Geranylgeranyl pyrophosphate synthase is a key enzyme in plant terpenoid, or isoprenoid, biosynthesis that catalyzes the synthesis of geranylgeranyl pyrophosphate by the addition of isopentenyl pyrophosphate to an allylic pyrophosphate. SEQ ID NO:84 sets forth the amino acid sequence of an Arabidopsis clone, identified herein as Ceres cDNA ID no. 7089429 (SEQ ID NO:83), that is predicted to encode a geranylgeranyl pyrophosphate synthase polypeptide containing a polyprenyl_synt domain.

A protein-modulating polypeptide can comprise the amino acid sequence set forth in SEQ ID NO:84. Alternatively, a protein-modulating polypeptide can be a homolog, ortholog, or variant of the polypeptide having the amino acid sequence set forth in SEQ ID NO:84. For example, a protein-modulating polypeptide can have an amino acid sequence with at least 40% sequence identity, e.g., 41%, 45%, 50%, 55%, 61%, 65%, 70%, 75%, 80%, 85%, 90%, 95%, 97%, 98%, or 99% sequence identity, to the amino acid sequence set forth in SEQ ID NO:84.

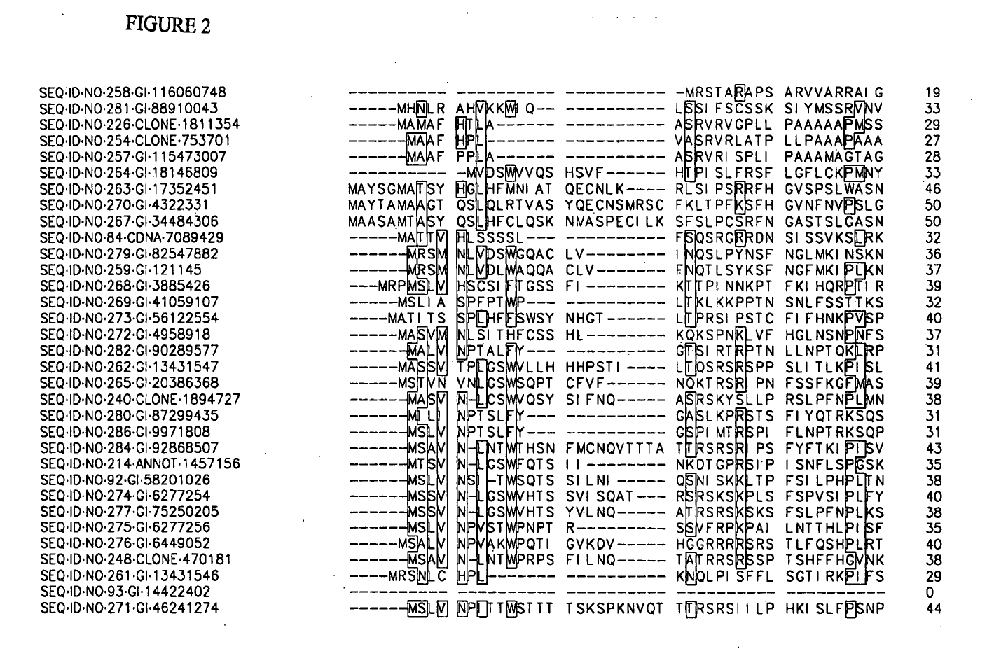

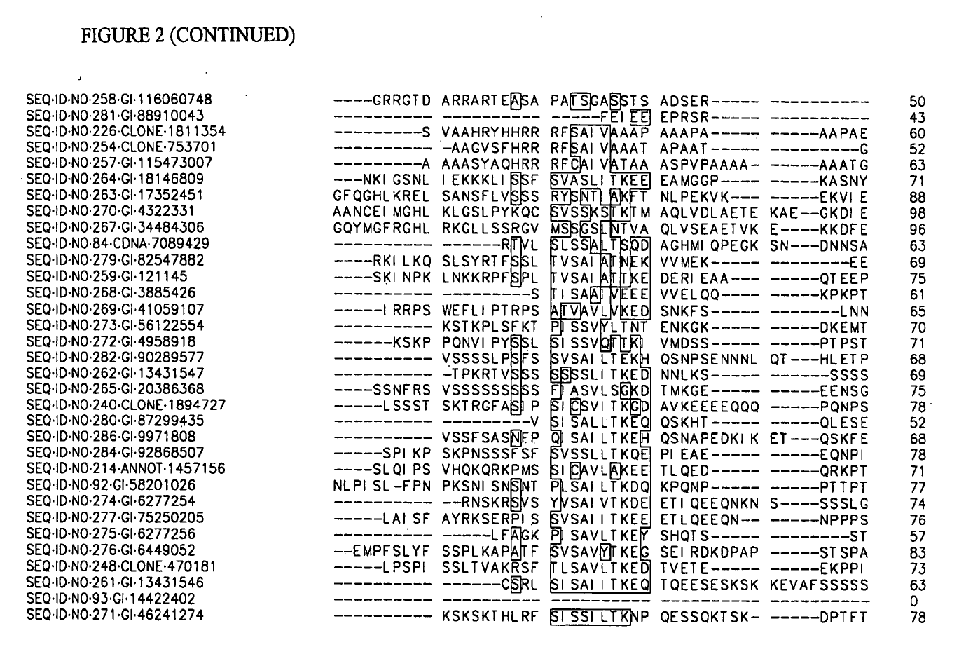

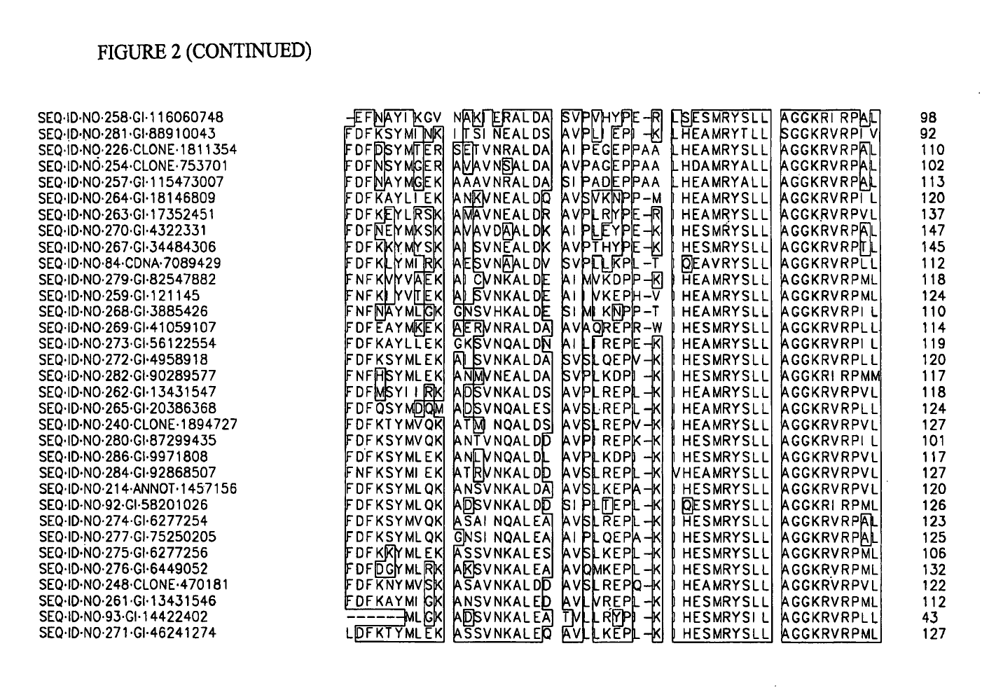

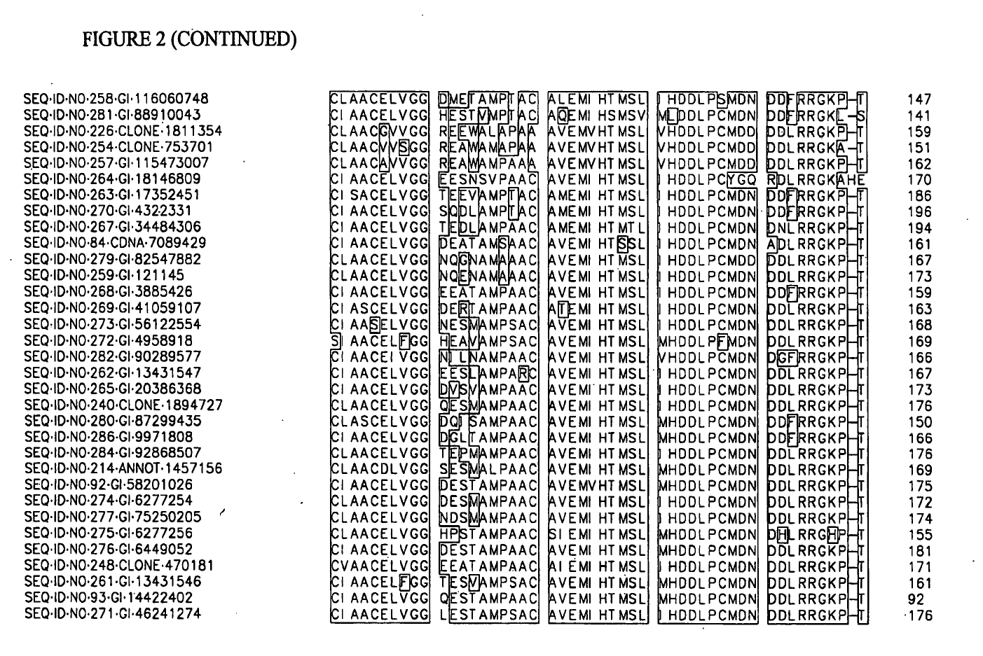

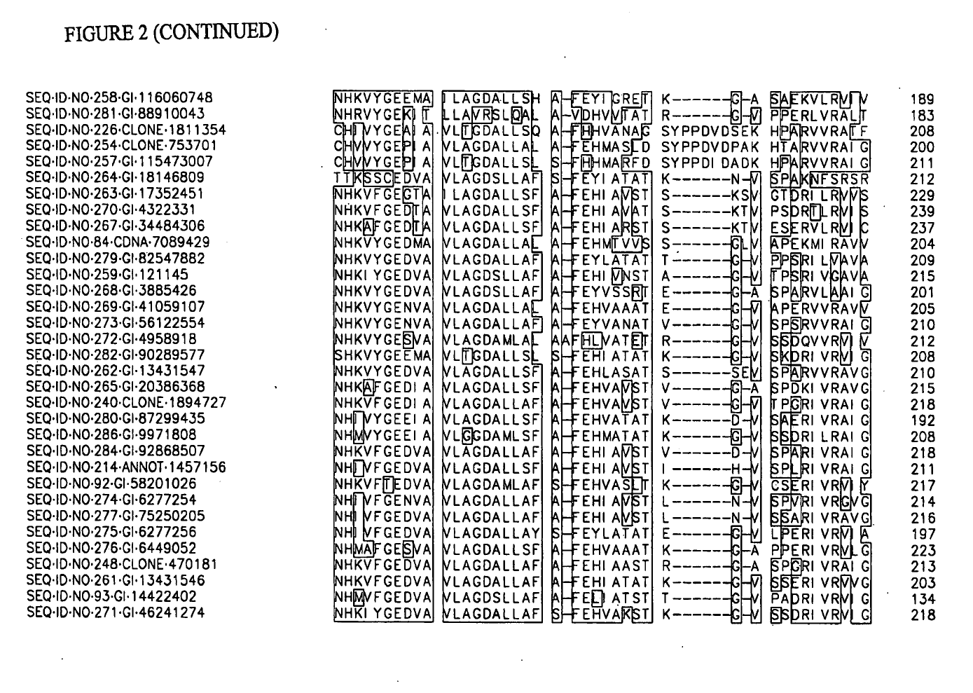

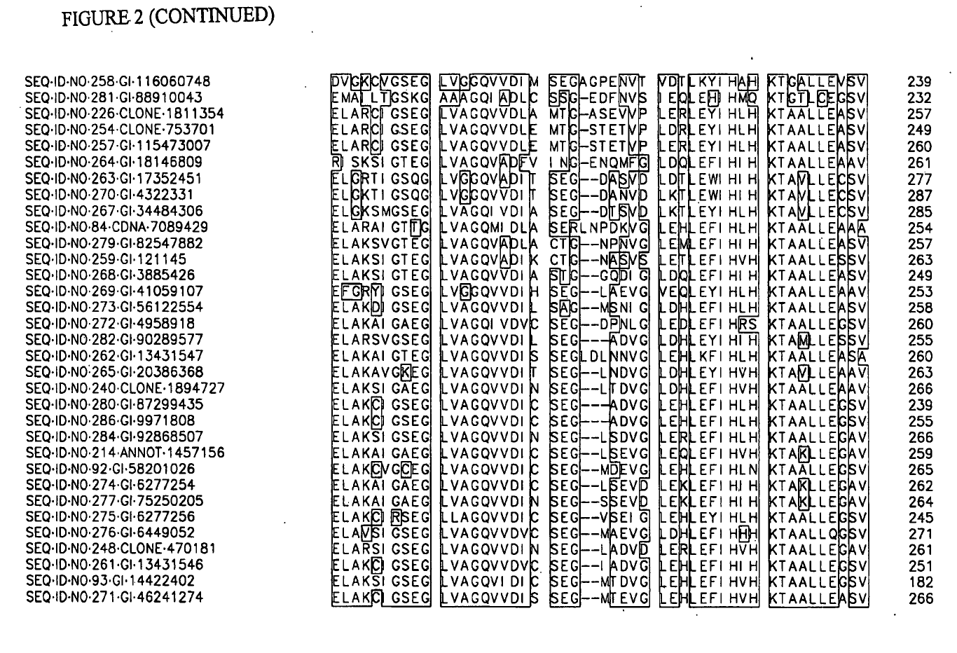

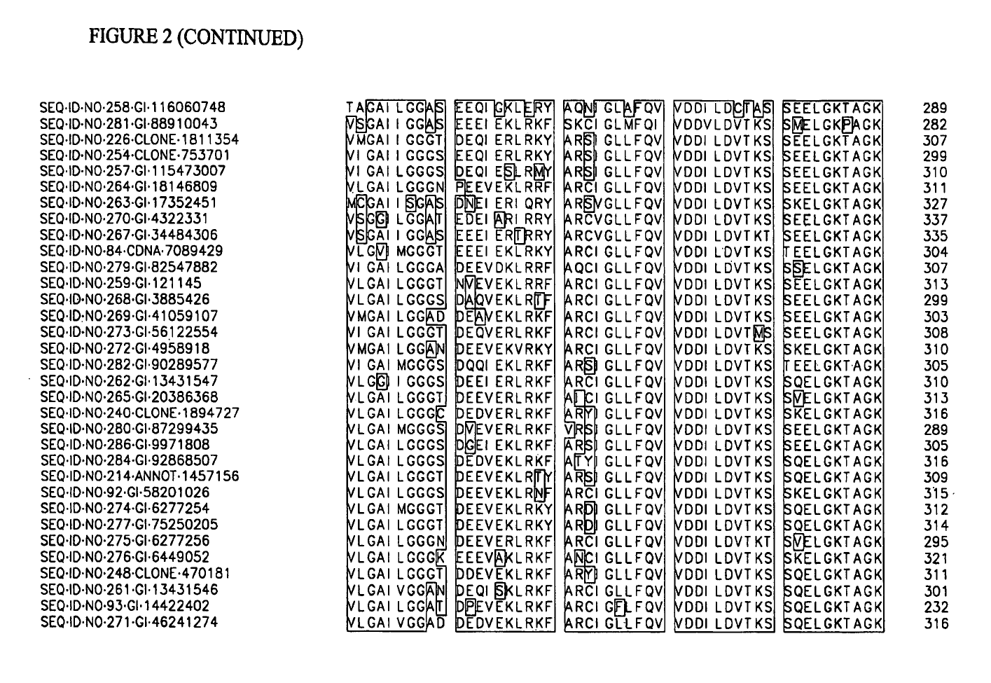

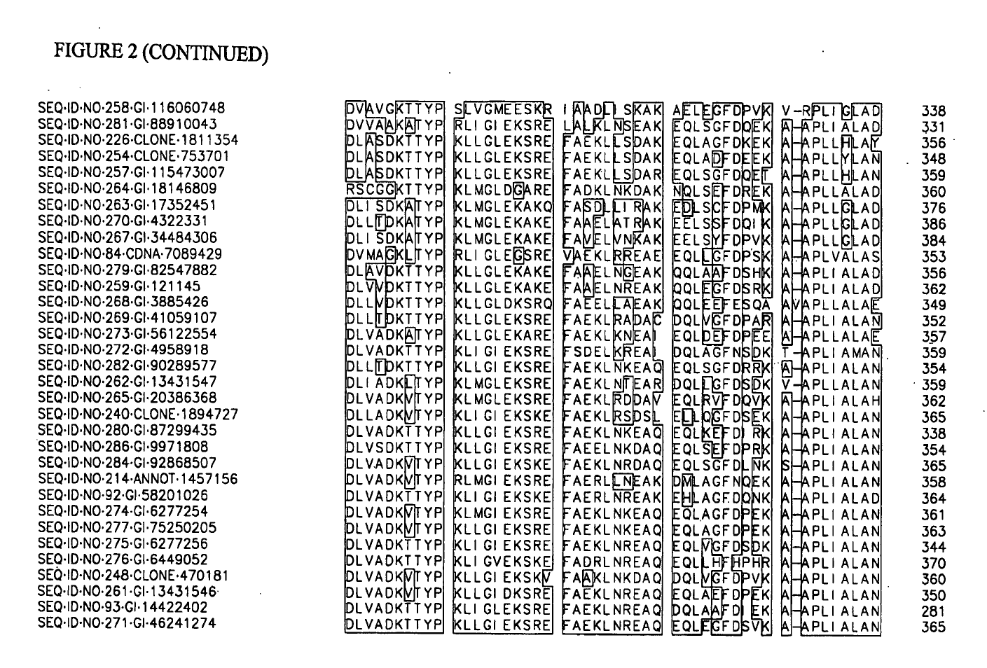

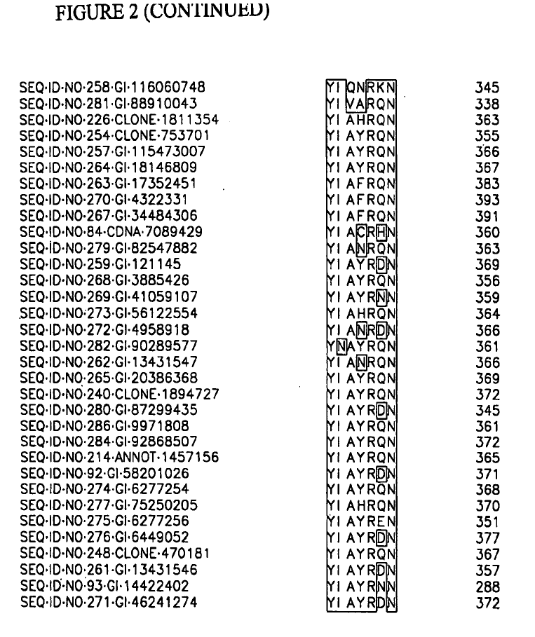

Amino acid sequences of homologs and/or orthologs of the polypeptide having the amino acid sequence set forth in SEQ ID NO:84 are provided in FIG. 2. The alignment in FIG. 2 provides the amino acid sequences of cDNA 7089429 (SEQ ID NO:84), GI 58201026 (SEQ ID NO:92), GI 14422402 (SEQ ID NO:93), ANNOT 1457156 (SEQ ID NO:214), CLONE 1811354 (SEQ ID NO:226), CLONE 1894727 (SEQ ID NO:240), CLONE 470181 (SEQ ID NO:248), CLONE 753701 (SEQ ID NO:254), GI 115473007 (SEQ ID NO:257), GI 116060748 (SEQ ID NO:258), GI 121145 (SEQ ID NO:259), GI 13431546 (SEQ ID NO:261), GI 13431547 (SEQ ID NO:262), G117352451 (SEQ ID NO:263), GI 18146809 (SEQ ID NO:264), GI 20386368 (SEQ ID NO:265), GI 34484306 (SEQ ID NO:267), GI 3885426 (SEQ ID NO:268), GI 41059107 (SEQ ID NO:269), GI 4322331 (SEQ ID NO:270), GI 462-41274 (SEQ ID NO:271), GI 4958918 (SEQ ID NO:272), GI 56122554 (SEQ ID NO:273), GI 6277254 (SEQ ID NO:274), G16277256 (SEQ ID NO:275), GI 6449052 (SEQ ID NO:276), GI 75250205 (SEQ ID NO:277), GI 82547882 (SEQ ID NO:279), GI 87299435 (SEQ ID NO:280), GI 88910043 (SEQ ID NO:281), GI 90289577 (SEQ ID NO:282), GI 92868507 (SEQ ID NO:284), and GI 9971808 (SEQ ID NO:286). Other homologs and/or orthologs include Public GI no. 26450928 (SEQ ID NO:85), Public GI no. 21592547 (SEQ ID NO:87), Public GI no. 11994525 (SEQ ID NO:88), Ceres CLONE ID no. 117906 (SEQ ID NO:89), Public GI no. 50253560 (SEQ ID NO:90), Public GI no. 62320250 (SEQ ID NO:91), Ceres ANNOT ID no. 1487885 (SEQ ID NO:217), Public GI ID no. 22535957 (SEQ ID NO:266), and Public GI ID no. 79154586 (SEQ ID NO:278).

In some cases, a protein-modulating polypeptide includes a polypeptide having at least 80% sequence identity, e.g., 80%, 83%, 85%, 86%, 87%, 88%, 89%, 90%, 91%, 92%, 93%, 94%, 95%, 96%, 97%, 98%, or 99% sequence identity, to an amino acid sequence corresponding to SEQ ID NO:85, SEQ ID NO:87, SEQ ID NO:88, SEQ ID NO:89, SEQ ID NO:90, SEQ ID NO:91, SEQ ID NO:92, SEQ ID NO:93, SEQ ID NO:214, SEQ ID NO:217, SEQ ID NO:226, SEQ ID NO:240, SEQ ID NO:248, SEQ ID NO:254, SEQ ID NO:257, SEQ ID NO:258, SEQ ID NO:259, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, SEQ ID NO:282, SEQ ID NO:284, or SEQ ID NO:286.

A protein-modulating polypeptide can contain a WD-40 repeat. WD-40 repeats, also known as WD or beta-transducin repeats, are motifs consisting of about 40 amino acids that often terminate in a Trp-Asp (W-D) dipeptide. Polypeptides containing WD repeats have 4 to 16 repeating units, which are thought to form a circularized beta-propeller structure. WD-repeat polypeptides serve as an assembly platform for multiprotein complexes in which the repeating units serve as a rigid scaffold for polypeptide interactions. Examples of such complexes include G protein complexes, the beta subunits of which are beta-propellers; TAFII transcription factor complexes; and E3 ubiquitin ligase complexes. WD-repeat polypeptides form a large family of eukaryotic polypeptides implicated in a variety of functions ranging from signal transduction and transcription regulation to cell cycle control and apoptosis. SEQ ID NO:95 sets forth the amino acid sequence of a Zea mays clone, identified herein as Ceres CLONE ID no. 285705 (SEQ ID NO:94), that is predicted to encode a WD-repeat polypeptide.

A protein-modulating polypeptide can comprise the amino acid sequence set forth in SEQ ID NO:95. Alternatively, a protein-modulating polypeptide can be a homolog, ortholog, or variant of the polypeptide having the amino acid sequence set forth in SEQ ID NO:95. For example, a protein-modulating polypeptide can have an amino acid sequence with at least 45% sequence identity, e.g., 46%, 50%, 55%, 60%, 65%, 70%, 75%, 80%, 85%, 90%, 95%, 97%, 98%, or 99% sequence identity, to the amino acid sequence set forth in SEQ ID NO:95.

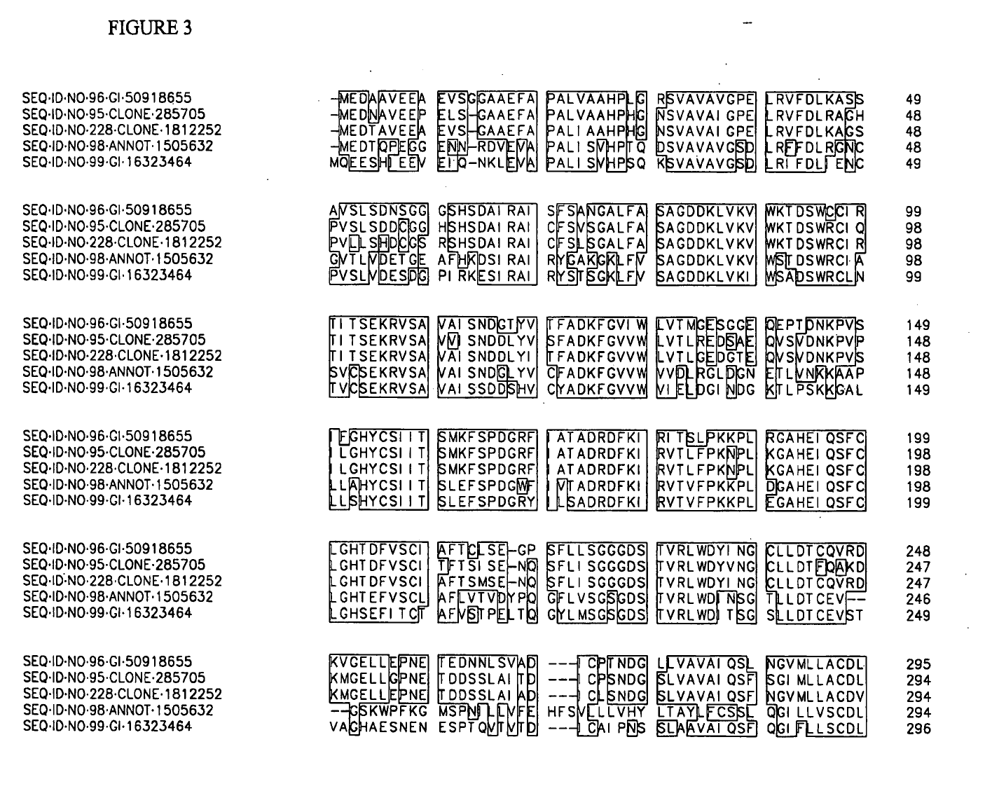

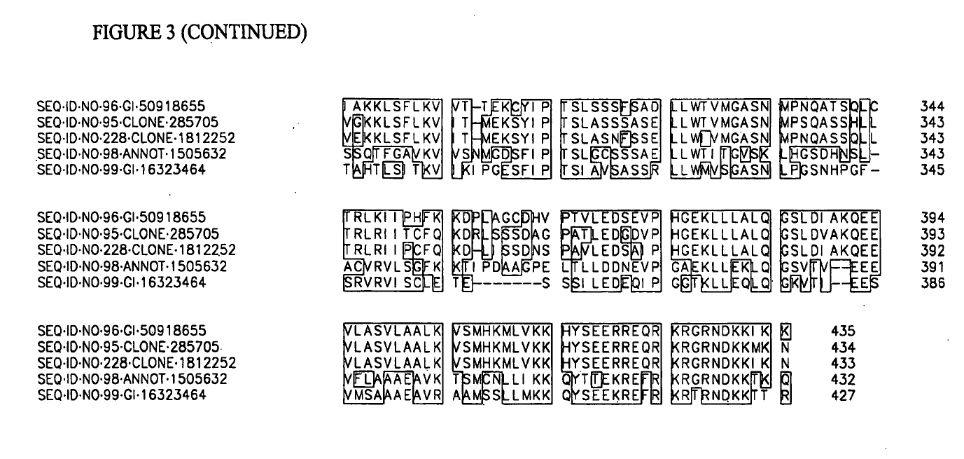

Amino acid sequences of homologs and/or orthologs of the polypeptide having the amino acid sequence set forth in SEQ ID NO:95 are provided in FIG. 3. The alignment in FIG. 3 provides the amino acid sequences of CLONE 285705 (SEQ ID NO:95), GI 50918655 (SEQ ID NO:96), ANNOT 1505632 (SEQ ID NO:98), GI 16323464 (SEQ ID NO:99), and CLONE 1812252 (SEQ ID NO:228). Other homologs and/or orthologs include Ceres CLONE ID no. 3297 (SEQ ID NO:100).

In some cases, a protein-modulating polypeptide includes a polypeptide having at least 80% sequence identity, e.g., 80%, 83%, 85%, 86%, 87%, 88%, 89%, 90%, 91%, 92%, 93%, 94%, 95%, 96%, 97%, 98%, or 99% sequence identity, to an amino acid sequence corresponding to SEQ ID NO:96, SEQ ID NO:98, SEQ ID NO:99, SEQ ID NO:100, or SEQ ID NO:228.

A protein-modulating polypeptide can contain a leucine-rich repeat, such as LRR_{—}1. Leucine-rich repeats (LRR) consist of 2-45 motifs of 20-30 amino acids that generally fold into an arc or horseshoe shape and are often flanked by cysteine rich domains. Each LRR is composed of a beta-alpha unit. LRRs appear to provide a structural framework for the formation of protein-protein interactions. Polypeptides containing LRRs include tyrosine kinase receptors, cell-adhesion molecules, virulence factors, and extracellular matrix-binding glycoproteins that are involved in a variety of biological processes, including signal transduction, cell adhesion, DNA repair, recombination, transcription, RNA processing, and disease resistance. SEQ ID NO:112 sets forth the amino acid sequence of an Arabidopsis clone, identified herein as Ceres cDNA ID no. 12720115 (SEQ ID NO:111), that is predicted to encode a polypeptide containing a leucine-rich repeat.

A protein-modulating polypeptide can comprise the amino acid sequence set forth in SEQ ID NO:112. Alternatively, a protein-modulating polypeptide can be a homolog, ortholog, or variant of the polypeptide having the amino acid sequence set forth in SEQ ID NO:112. For example, a protein-modulating polypeptide can have an amino acid sequence with at least 40% sequence identity, e.g., 41%, 45%, 50%, 55%, 60%, 65%, 70%, 75%, 80%, 85%, 90%, 95%, 97%, 98%, or 99% sequence identity, to the amino acid sequence set forth in SEQ ID NO:112.

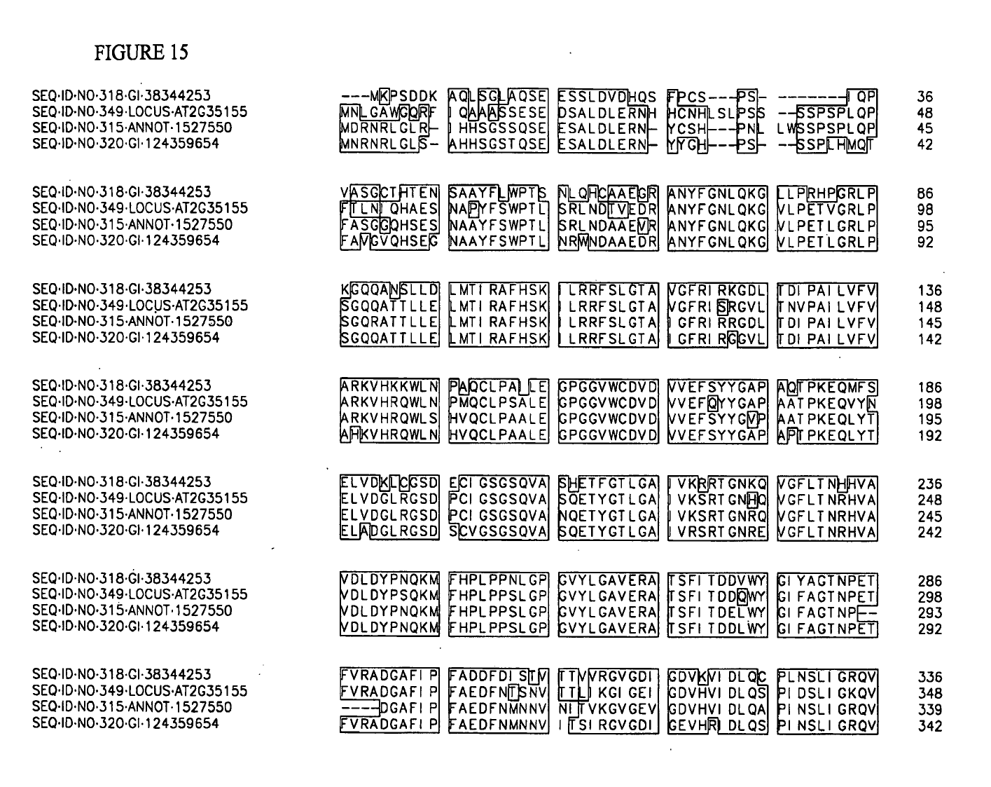

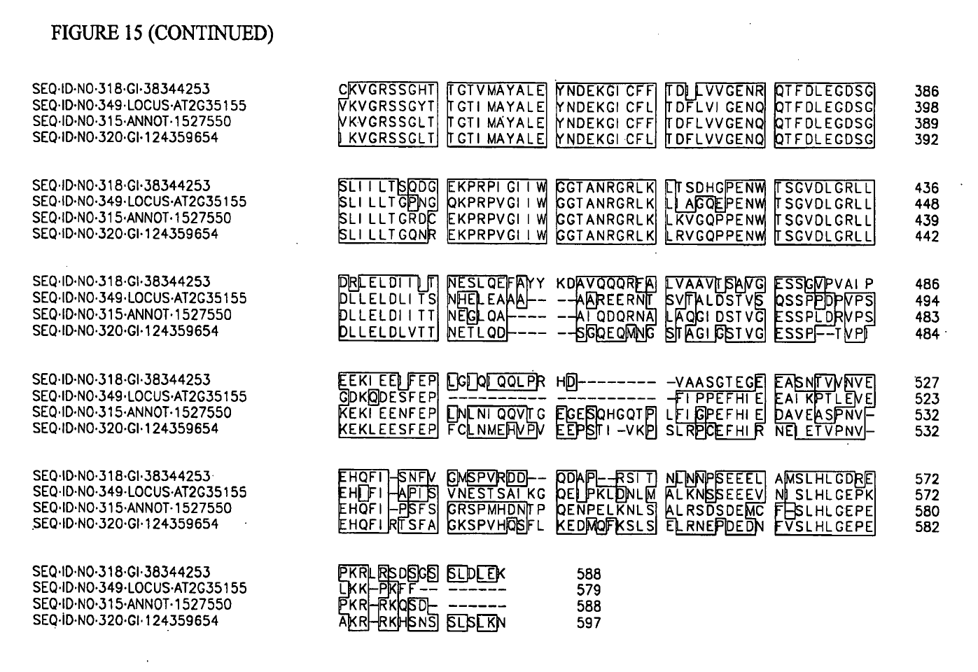

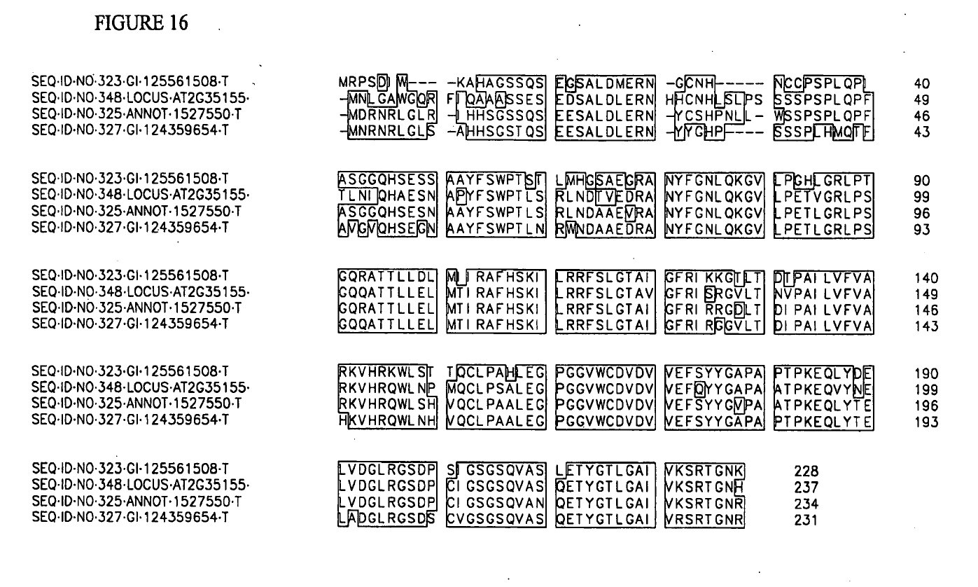

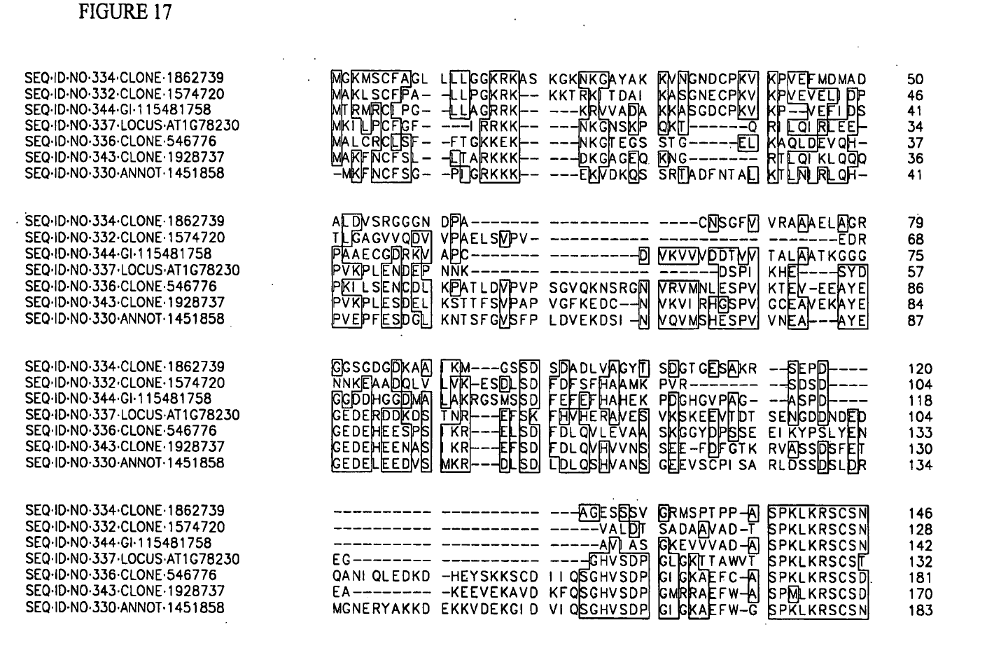

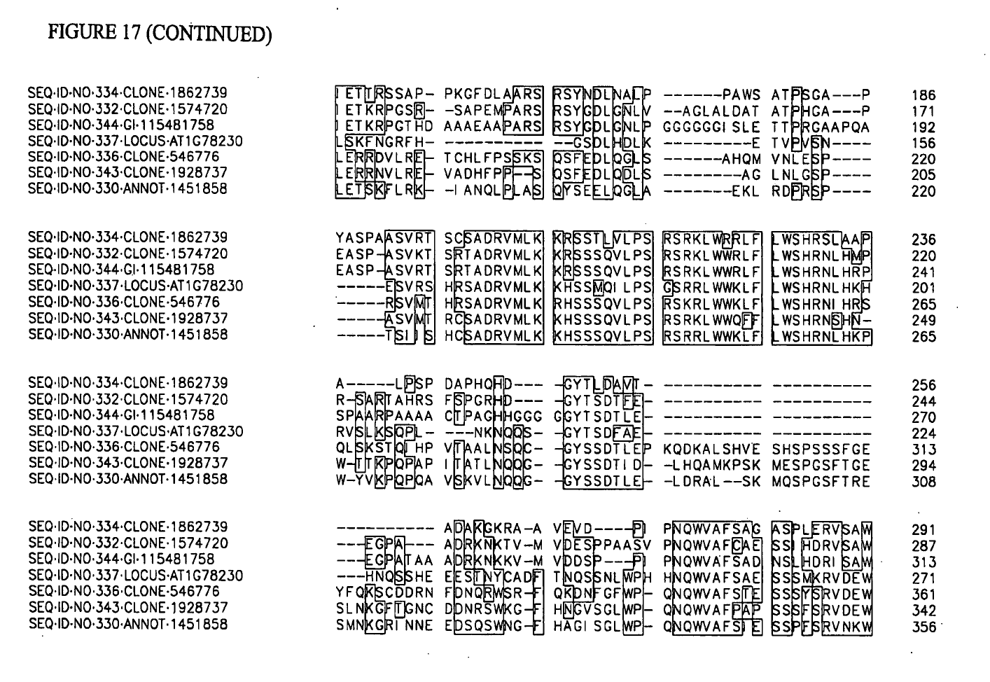

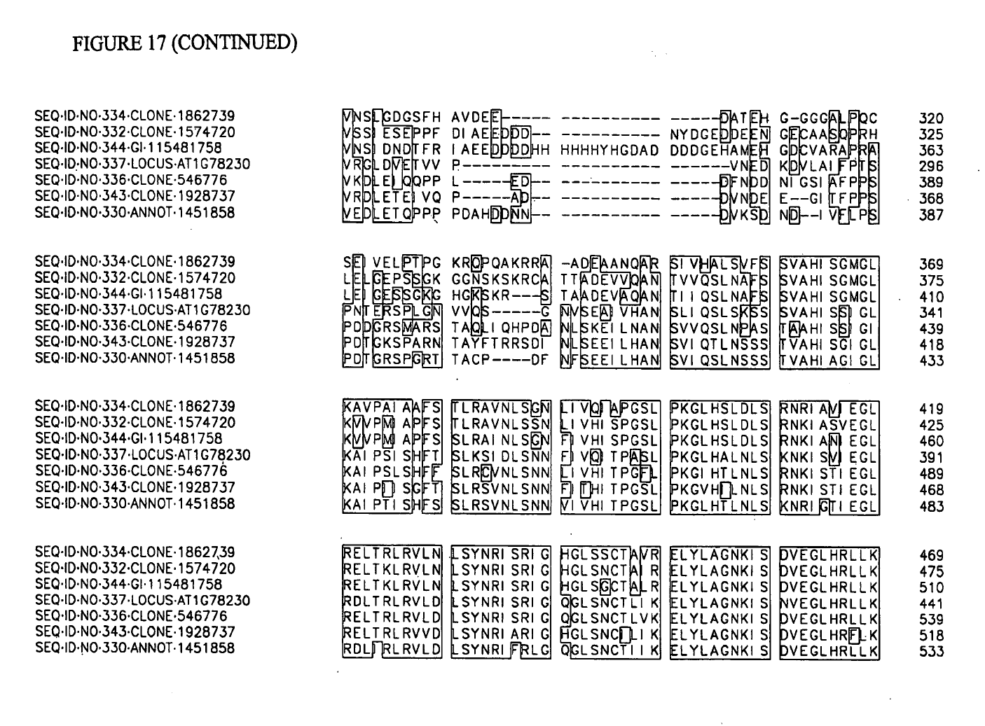

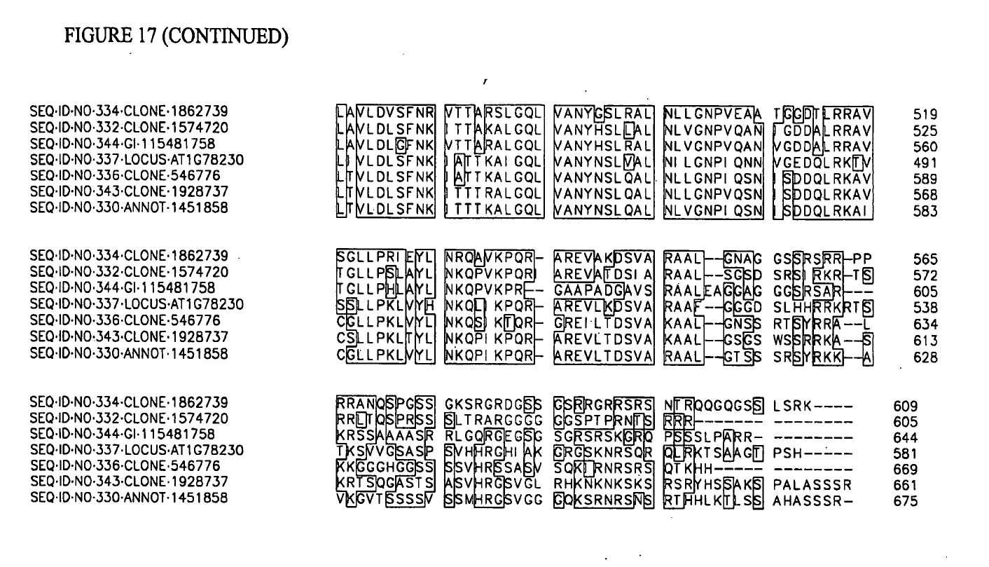

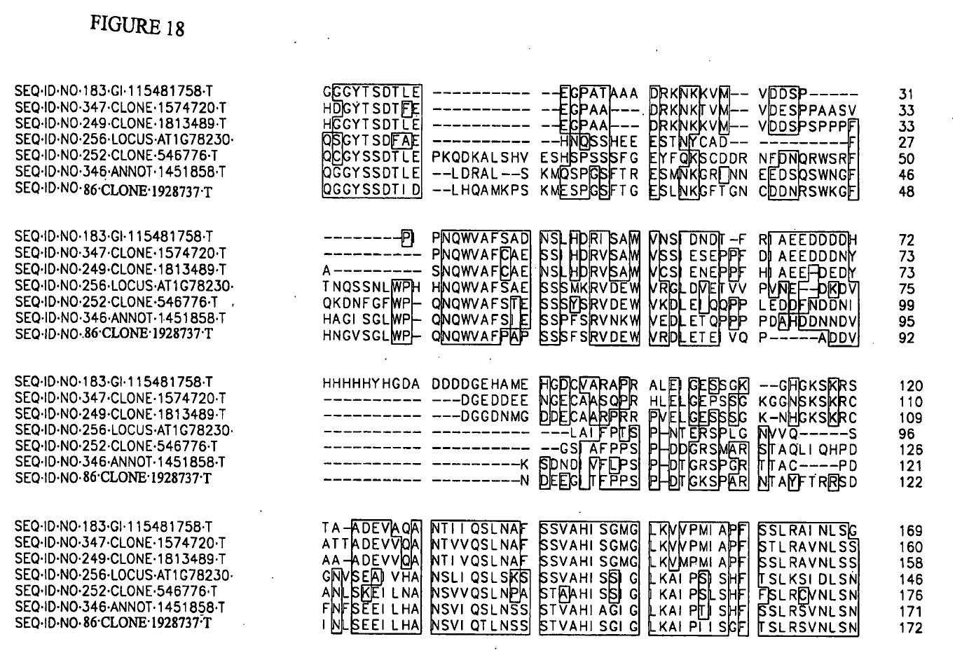

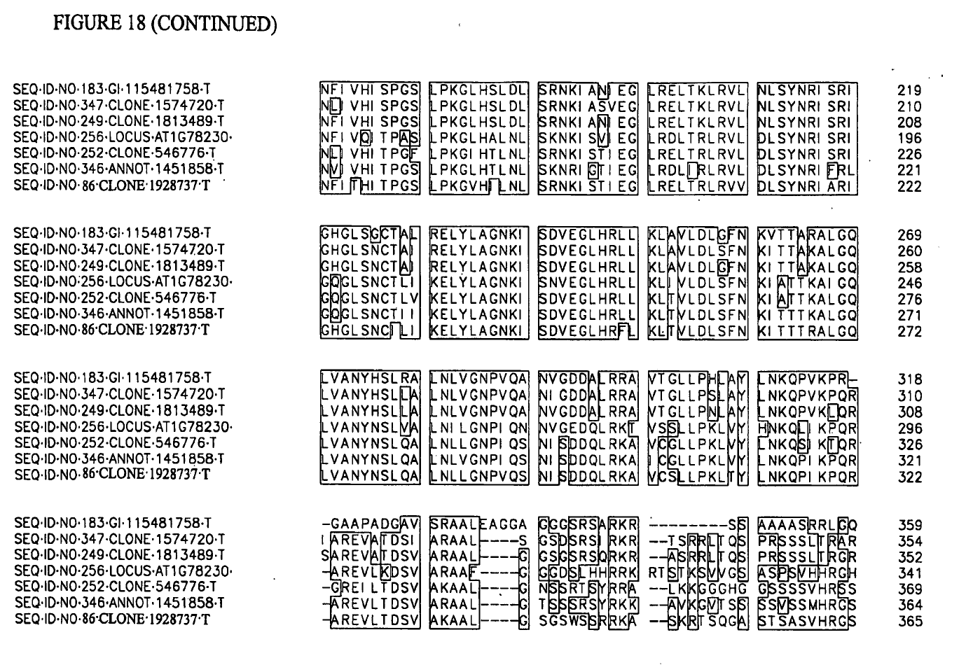

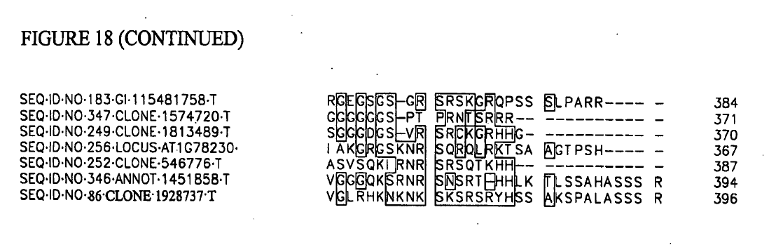

Amino acid sequences of homologs and/or orthologs of the polypeptide having the amino acid sequence set forth in SEQ ID NO:112 are provided in FIGS. 15, 16, 17, and 18. The alignment in FIG. 15 provides the amino acid sequences of LOCUS AT2G35155 (SEQ ID NO:349), ANNOT 1527550 (SEQ ID NO:315), GI 38344253 (SEQ ID NO:318), and GI 124359654 (SEQ ID NO:320). The alignment in FIG. 16 provides the amino acid sequences of LOCUS AT2G35155·T (SEQ ID NO:348), GI 125561508·T (SEQ ID NO:323), ANNOT 1527550·T (SEQ ID NO:325), and GI 124359654·T (SEQ ID NO:327). The alignment in FIG. 17 provides the amino acid sequences of LOCUS ATlG78230 (SEQ ID NO:337), ANNOT 1451858 (SEQ ID NO:330), CLONE 1574720 (SEQ ID NO:332), CLONE 1862739 (SEQ ID NO:334), CLONE 546776 (SEQ ID NO:336), CLONE 1928737 (SEQ ID NO:343), and GI 115481758 (SEQ ID NO:344). The alignment in FIG. 18 provides the amino acid sequences of LOCUS AT1G78230·T (SEQ ID NO:256), ANNOT 1451858·T (SEQ ID NO:346), CLONE 1574720·T (SEQ ID NO:347), CLONE 1928737·T (SEQ ID NO:86), GI 115481758·T (SEQ ID NO:183), CLONE 1813489·T (SEQ ID NO:249), and CLONE 546776·T (SEQ ID NO:252).

Other homologs and/or orthologs include Ceres GI ID no. 125574597_T (SEQ ID NO:215), Ceres CLONE ID no. 1407377_T (SEQ ID NO:218), Ceres CLONE ID no. 1862739_T (SEQ ID NO:250), Ceres ANNOT ID no. 1537493 (SEQ ID NO:317), Public GI ID no. 115476358 (SEQ ID NO:319), Public GI ID no. 125561508 (SEQ ID NO:321), Public GI ID no. 115476358_T (SEQ ID NO:324), Ceres ANNOT ID no. 1537493_T (SEQ ID NO:326), Public GI ID no. 38344253_T (SEQ ID NO:328), Ceres CLONE ID no. 1407377 (SEQ ID NO:339), Ceres CLONE ID no. 1813489 (SEQ ID NO:341), and Public GI ID no. 125574597 (SEQ ID NO:345).

In some cases, a protein-modulating polypeptide includes a polypeptide having at least 80% sequence identity, e.g., 80%, 83%, 85%, 86%, 87%, 88%, 89%, 90%, 91%, 92%, 93%, 94%, 95%, 96%, 97%, 98%, or 99% sequence identity, to an amino acid sequence corresponding to any of SEQ ID NO:86, SEQ ID NO:183, SEQ ID NO:215, SEQ ID NO:218, SEQ ID NO:249, SEQ ID NO:250, SEQ ID NO:252, SEQ ID NO:256, SEQ ID NO:315, SEQ ID NO:317, SEQ ID NO:318, SEQ ID NO:319, SEQ ID NO:320, SEQ ID NO:321, SEQ ID NO:323, SEQ ID NO:324, SEQ ID NO:325, SEQ ID NO:326, SEQ ID NO:327, SEQ ID NO:328, SEQ ID NO:330, SEQ ID NO:332, SEQ ID NO:334, SEQ ID NO:336, SEQ ID NO:337, SEQ ID NO:339, SEQ ID NO:341, SEQ ID NO:343, SEQ ID NO:344, SEQ ID NO:345, SEQ ID NO:346, SEQ ID NO:347, SEQ ID NO:348, or SEQ ID NO:349.

A protein-modulating polypeptide can be a kinase polypeptide, such as a 3-phosphoinositide-dependent protein kinase-1 polypeptide. A 3-phosphoinositide-dependent protein kinase-1 polypeptide catalyzes the following reaction: ATP+a protein=ADP+a phosphoprotein. The activity of a 3-phosphoinositide-dependent protein kinase-1 polypeptide is dependent on the presence of a 3-phosphoinositide lipid. A plant homologue of mammalian 3-phosphoinositide-dependent protein kinase-1 has been identified in Arabidopsis and rice which is reported to display 40% overall identity to human 3-phosphoinositide-dependent protein kinase-1. Like the mammalian 3-phosphoinositide-dependent protein kinase-1, Arabidopsis 3-phosphoinositide-dependent protein kinase-1 and rice 3-phosphoinositide-dependent protein kinase-1 possess an N-terminal kinase domain and a C-terminal pleckstrin homology domain. Arabidopsis 3-phosphoinositide-dependent protein kinase-1 can rescue lethality in Saccharomyces cerevisiae caused by disruption of genes encoding yeast 3-phosphoinositide-dependent protein kinase-1 homologues. Arabidopsis 3-phosphoinositide-dependent protein kinase-1 interacts via its pleckstrin homology domain with phosphatidic acid, PtdIns3P, PtdIns(3,4,5)P3 and PtdIns(3,4)P2 and to a lesser extent with PtdIns(4,5)P2 and PtdIns4P. Arabidopsis 3-phosphoinositide-dependent protein kinase-1 is able to activate human protein kinase B alpha (PKB/AKT) in the presence of PtdIns(3,4,5)P3. SEQ ID NO:114 sets forth the amino acid sequence of an Arabidopsis clone, identified herein as Ceres cDNA ID no. 23416880 (SEQ ID NO:113), that is predicted to encode a 3-phosphoinositide-dependent protein kinase-1 polypeptide.

A protein-modulating polypeptide can comprise the amino acid sequence set forth in SEQ ID NO:114. Alternatively, a protein-modulating polypeptide can be a homolog, ortholog, or variant of the polypeptide having the amino acid sequence set forth in SEQ ID NO:114. For example, a protein-modulating polypeptide can have an amino acid sequence with at least 40% sequence identity, e.g., 40%, 45%, 50%, 55%, 60%, 65%, 70%, 75%, 80%, 85%, 90%, 95%, 97%, 98%, or 99% sequence identity, to the amino acid sequence set forth in SEQ ID NO:114.

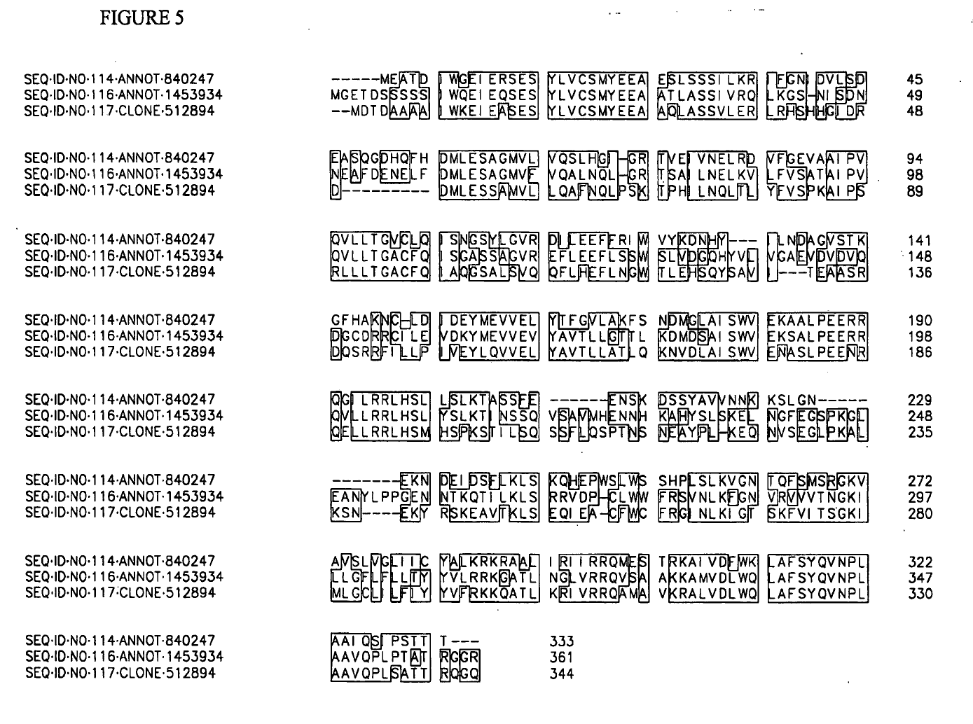

Amino acid sequences of homologs and/or orthologs of the polypeptide having the amino acid sequence set forth in SEQ ID NO:114 are provided in FIG. 5. The alignment in FIG. 5 provides the amino acid sequences of ANNOT 840247 (SEQ ID NO:114), ANNOT 1453934 (SEQ ID NO:116) and CLONE 512894 (SEQ ID NO:117).

In some cases, a protein-modulating polypeptide includes a polypeptide having at least 80% sequence identity, e.g., 80%, 83%, 85%, 86%, 87%, 88%, 89%, 90%, 91%, 92%, 93%, 94%, 95%, 96%, 97%, 98%, or 99% sequence identity, to an amino acid sequence corresponding to SEQ ID NO:116 or SEQ ID NO:117.

A protein-modulating polypeptide can contain a zf-CCHC domain characteristic of a zinc knuckle polypeptide. The zinc knuckle is a zinc binding motif with the sequence CX2CX4HX4C, where X can be any amino acid. The motifs are common to the nucleocapsid proteins of retroviruses, and the prototype structure is from HIV. The zinc knuckle family also contains members involved in eukaryotic gene regulation. A zinc knuckle is found in eukaryotic proteins involved in RNA binding or single strand DNA binding. SEQ ID NO:130 sets forth the amino acid sequence of an Arabidopsis clone, identified herein as Ceres cDNA ID no. 13579142 (SEQ ID NO:129), that is predicted to encode a polypeptide having a zf-CCHC domain.

A protein-modulating polypeptide can comprise the amino acid sequence set forth in SEQ ID NO:130. Alternatively, a protein-modulating polypeptide can be a homolog, ortholog, or variant of the polypeptide having the amino acid sequence set forth in SEQ ID NO:130. For example, a protein-modulating polypeptide can have an amino acid sequence with at least 45% sequence identity, e.g., 46%, 50%, 55%, 60%, 65%, 70%, 75%, 80%, 85%, 90%, 95%, 97%, 98%, or 99% sequence identity, to the amino acid sequence set forth in SEQ ID NO:130.

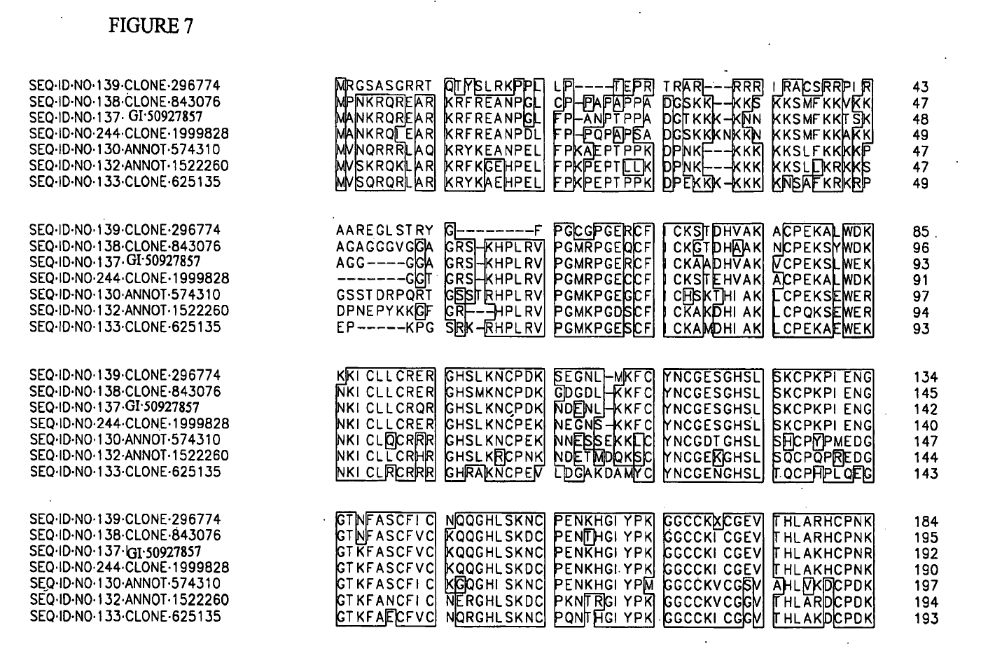

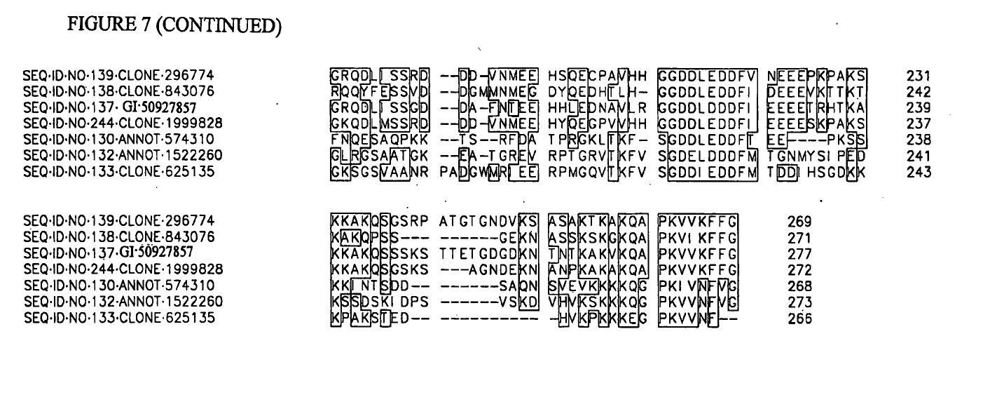

Amino acid sequences of homologs and/or orthologs of the polypeptide having the amino acid sequence set forth in SEQ ID NO:130 are provided in FIG. 7. The alignment in FIG. 7 provides the amino acid sequences of ANNOT 574310 (SEQ ID NO:130), ANNOT 1522260 (SEQ ID NO:132), CLONE 625135 (SEQ ID NO:133), GI 50927857(SEQ ID NO:137), CLONE 843076(SEQ ID NO:138), CLONE 296774 (SEQ ID NO:139), and CLONE 1999828 (SEQ ID NO:244). Other homologs and/or orthologs include Ceres GDNA ANNOT ID no. 1527806 (SEQ ID NO:135) and Ceres CLONE ID no. 463860 (SEQ ID NO:136).

In some cases, a protein-modulating polypeptide includes a polypeptide having at least 80% sequence identity, e.g., 80%, 83%, 85%, 86%, 87%, 88%, 89%, 90%, 91%, 92%, 93%, 94%, 95%, 96%, 97%, 98%, or 99% sequence identity, to an amino acid sequence corresponding to SEQ ID NO:132, SEQ ID NO:133, SEQ ID NO:135, SEQ ID NO:136, SEQ ID NO:137, SEQ ID NO:138, SEQ ID NO:139, or SEQ ID NO:244.

A protein-modulating polypeptide can contain a zf-C2H2 domain characteristic of C2H2 type zinc finger transcription factor polypeptides. Zinc finger domains are nucleic acid-binding polypeptide structures. The C2H2 zinc finger is the classical zinc finger domain. The two conserved cysteines and histidines coordinate a zinc ion. The following pattern describes the zinc finger: #X-C-X(1-5)-C-X3-#-X5-#-X2-H-X(3-6)-[H/C], where X can be any amino acid, the numbers in brackets indicate the number of residues, and the positions marked # are those that are important for the stable fold of the zinc finger. The final position can be either a histidine or cysteine residue. The C2H2 zinc finger is composed of two short beta strands followed by an alpha helix. The amino terminal part of the helix binds the major groove in DNA binding zinc fingers. C2H2 zinc finger family polypeptides play important roles in plant development including floral organogenesis, leaf initiation, lateral shoot initiation, gametogenesis, and seed development. SEQ ID NO:141 sets forth the amino acid sequence of a Brassica napus clone, identified herein as Ceres CLONE ID no. 1103471 (SEQ ID NO:140), that is predicted to encode a C2H2 zinc finger family polypeptide.

A protein-modulating polypeptide can comprise the amino acid sequence set forth in SEQ ID NO:141. Alternatively, a protein-modulating polypeptide can be a homolog, ortholog, or variant of the polypeptide having the amino acid sequence set forth in SEQ ID NO:141. For example, a protein-modulating polypeptide can have an amino acid sequence with at least 50% sequence identity, e.g., 51%, 55%, 60%, 65%, 70%, 75%, 80%, 85%, 90%, 95%, 97%, 98%, or 99% sequence identity, to the amino acid sequence set forth in SEQ ID NO:141.

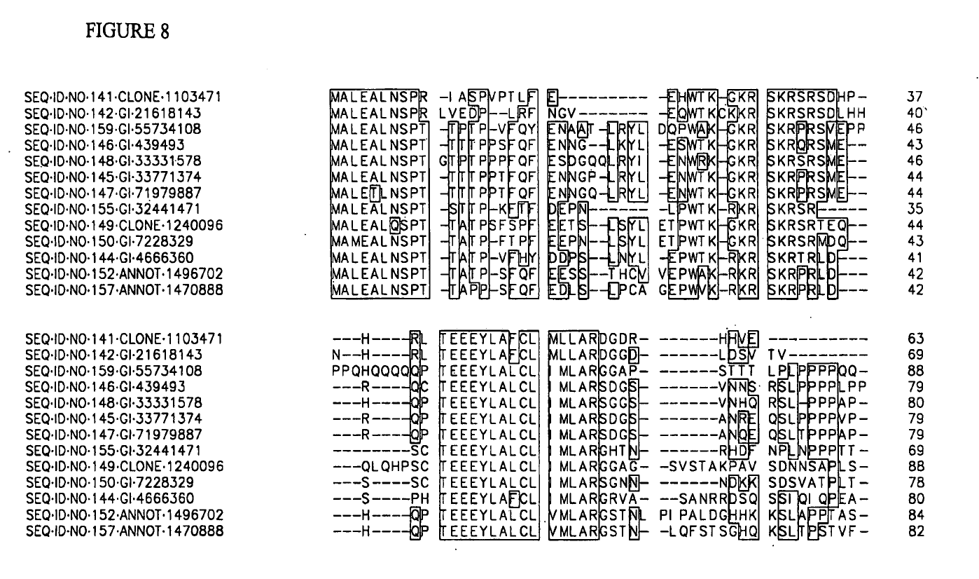

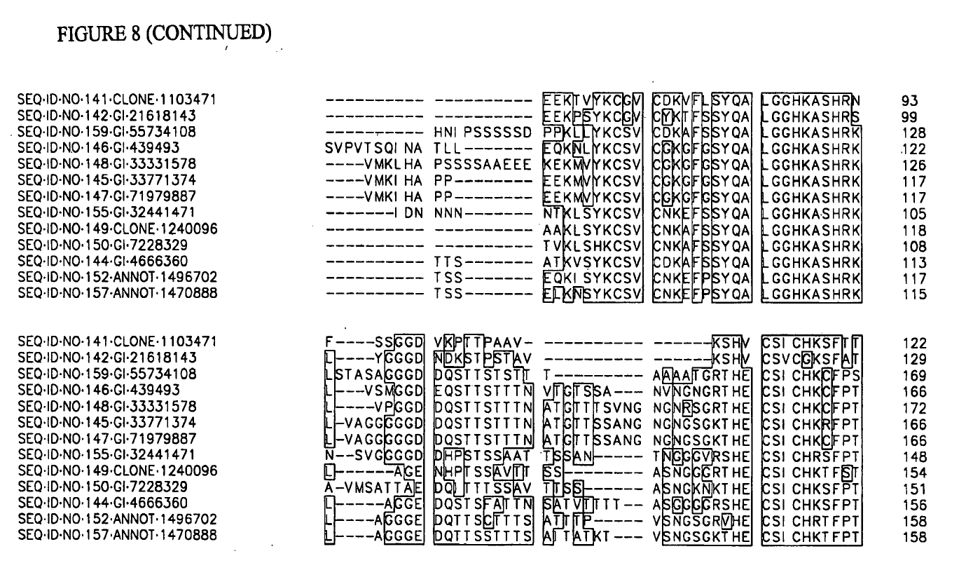

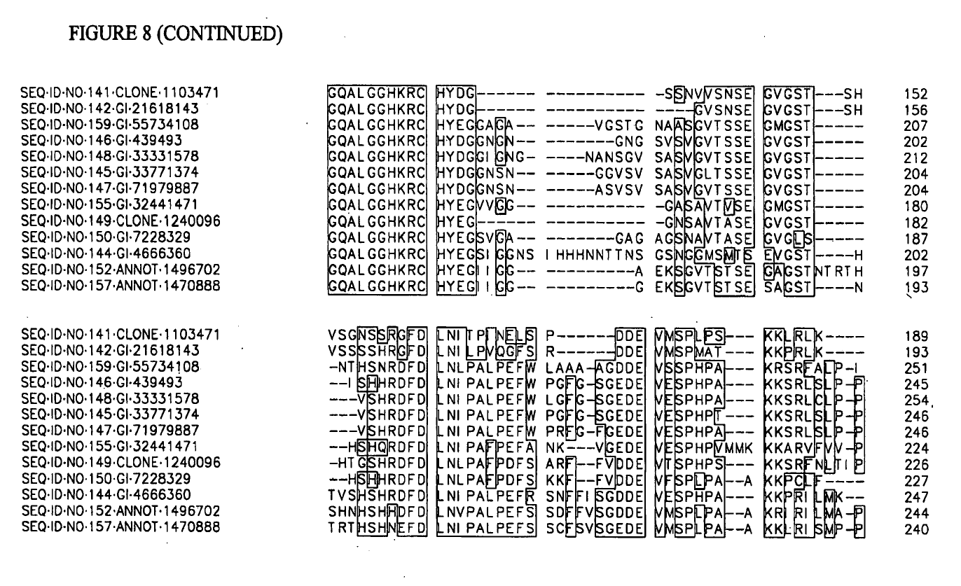

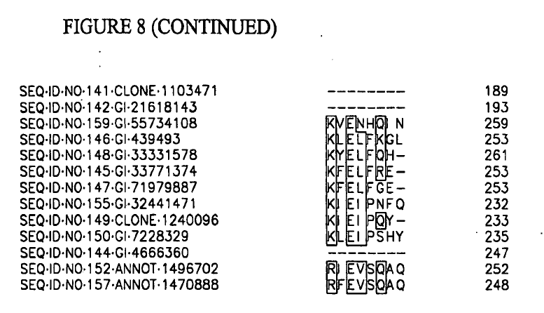

Amino acid sequences of homologs and/or orthologs of the polypeptide having the amino acid sequence set forth in SEQ ID NO:141 are provided in FIG. 8. The alignment in FIG. 8 provides the amino acid sequences of CLONE 1103471 (SEQ ID NO:141), GI 21618143 (SEQ ID NO:142), GI 4666360 (SEQ ID NO:144), GI 33771374 (SEQ ID NO:145), GI 439493 (SEQ ID NO:146), GI 71979887 (SEQ ID NO:147), GI 33331578 (SEQ ID NO:148), CLONE 1240096 (SEQ ID NO:149), GI 7228329 (SEQ ID NO:150), ANNOT 1496702 (SEQ ID NO:152), GI 32441471 (SEQ ID NO:155), ANNOT 1470888 (SEQ ID NO:157), and GI 55734108 (SEQ ID NO:159). Other homologs and/or orthologs include Public GI no. 6009889 (SEQ ID NO:143), Ceres GDNA ANNOT ID no. 1443763 (SEQ ID NO:154), and Ceres CLONE ID no. 1619683 (SEQ ID NO:158).

In some cases, a protein-modulating polypeptide includes a polypeptide having at least 80% sequence identity, e.g., 80%, 83%, 85%, 86%, 87%, 88%, 89%, 90%, 91%, 92%, 93%, 94%, 95%, 96%, 97%, 98%, or 99% sequence identity, to an amino acid sequence corresponding to SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:152, SEQ ID NO:154, SEQ ID NO:155, SEQ ID NO:157, SEQ ID NO:158, or SEQ ID NO:159.

A protein-modulating polypeptide can have a PI3_PI4_kinase domain characteristic of phosphatidylinositol 3- and 4-kinase polypeptides. Phosphatidylinositol 3-kinase (PI3-kinase) is an enzyme that phosphorylates phosphoinositides on the 3-hydroxyl group of the inositol ring. The three products of PI3-kinase, PI-3-P, PI-3,4-P(2), and PI-3,4,5-P(3), function as secondary messengers in cell signaling. Phosphatidylinositol 4-kinase (PI4-kinase) is an enzyme that acts on phosphatidylinositol (PI) in the first committed step in the production of the secondary messenger inositol-1,4,5,-trisphosphate. A PI3_PI4_kinase domain is also present in a wide range of protein kinases involved in diverse cellular functions, such as control of cell growth, regulation of cell cycle progression, regulation of the DNA damage checkpoint, recombination, and maintenance of telomere length. SEQ ID NO:161 sets forth the amino acid sequence of an Arabidopsis clone, identified herein as Ceres ANNOT ID no. 543117 (SEQ ID NO:160), that is predicted to encode a polypeptide containing a PI3_PI4_kinase domain.

A protein-modulating polypeptide can comprise the amino acid sequence set forth in SEQ ID NO:161. Alternatively, a protein-modulating polypeptide can be a homolog, ortholog, or variant of the polypeptide having the amino acid sequence set forth in SEQ ID NO:161. For example, a protein-modulating polypeptide can have an amino acid sequence with at least 50% sequence identity, e.g., 51%, 55%, 60%, 65%, 70%, 75%, 80%, 85%, 90%, 95%, 97%, 98%, or 99% sequence identity, to the amino acid sequence set forth in SEQ ID NO:161.

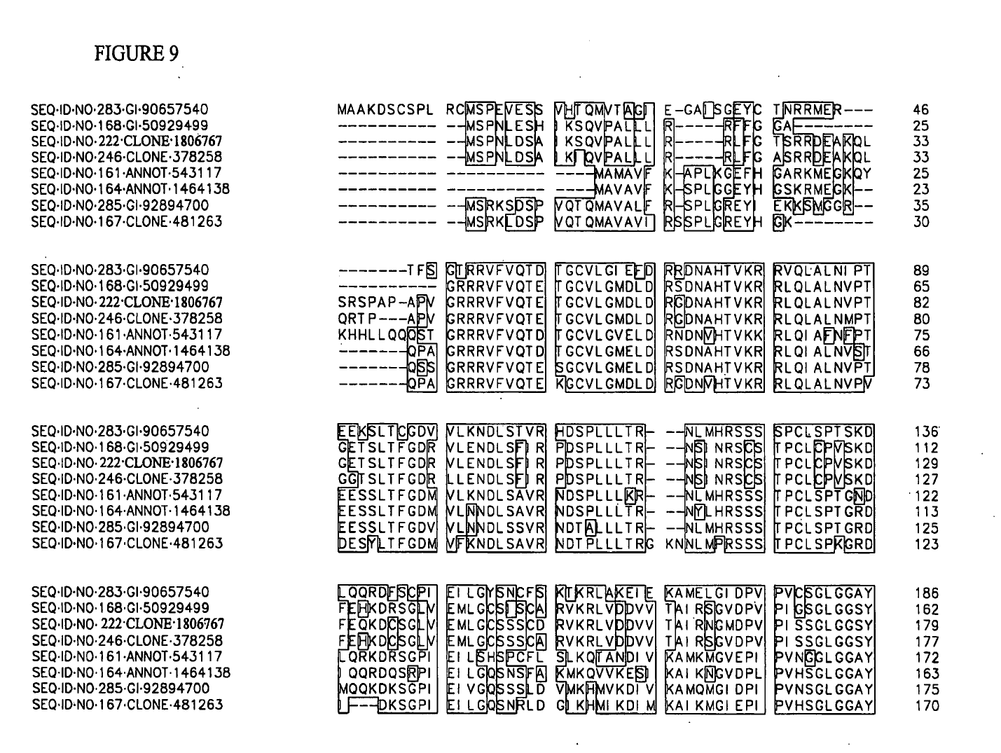

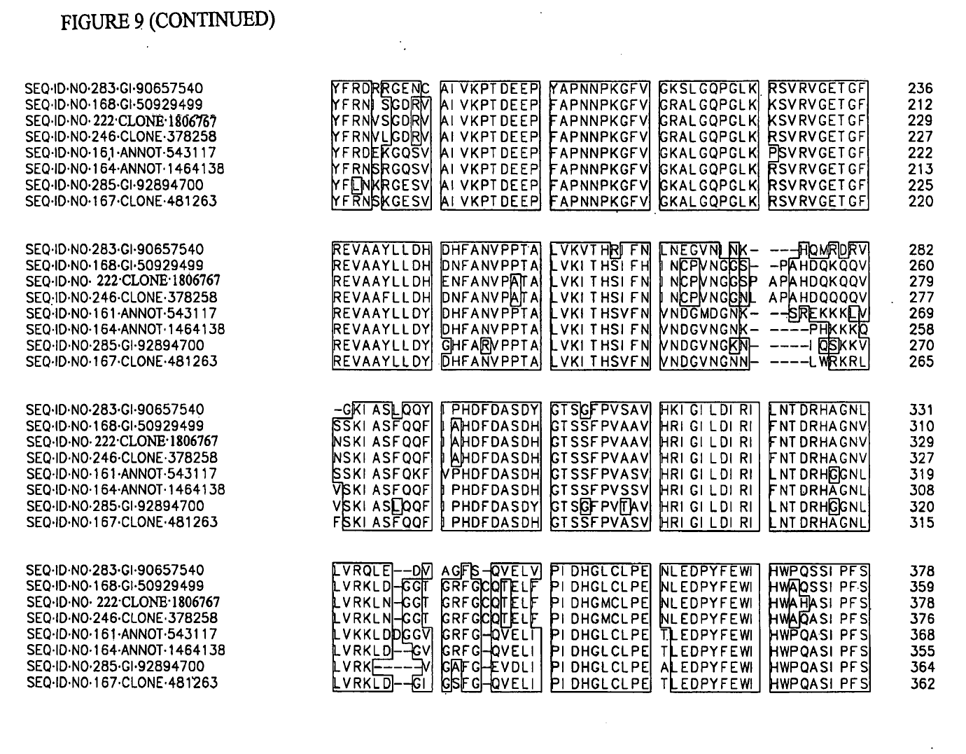

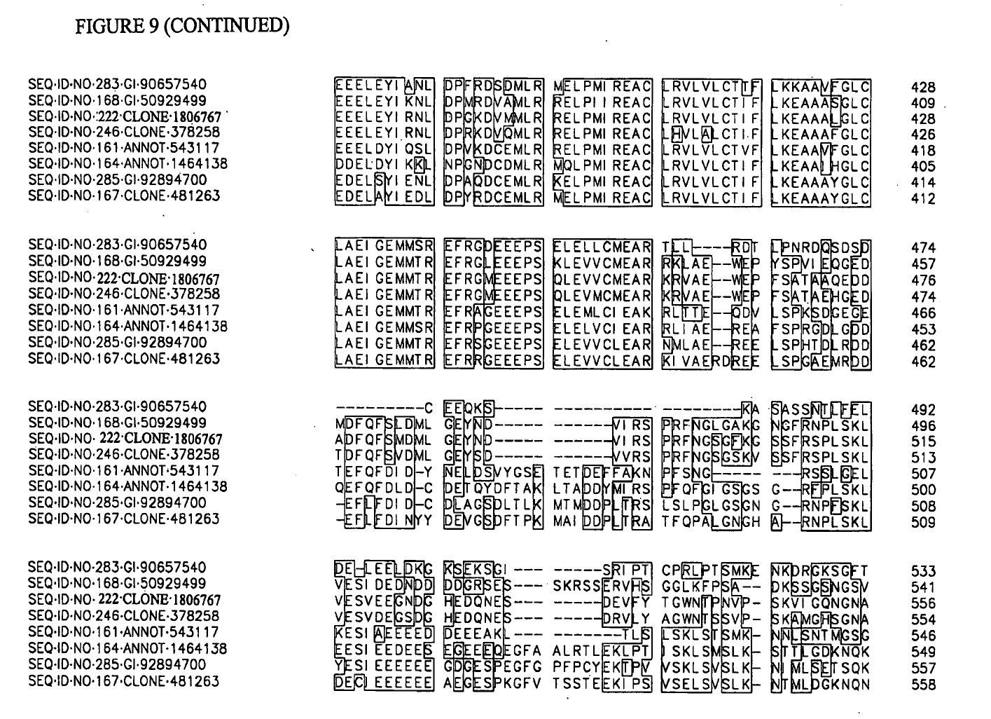

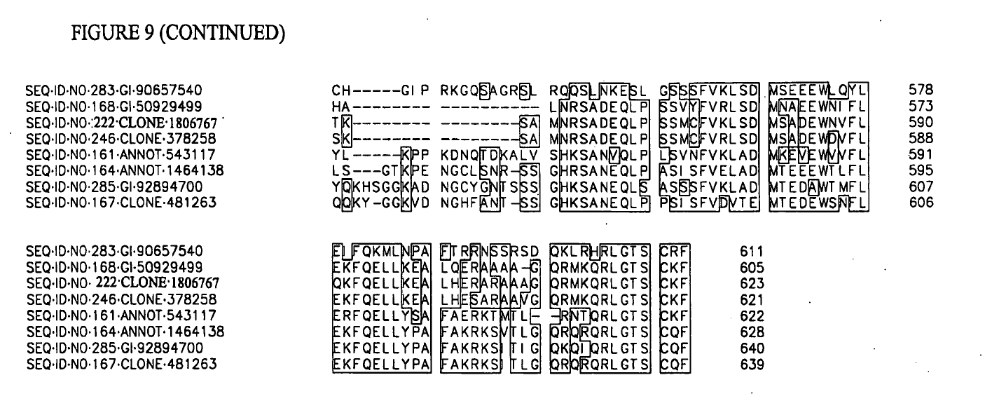

Amino acid sequences of homologs and/or orthologs of the polypeptide having the amino acid sequence set forth in SEQ ID NO:161 are provided in FIG. 9. The alignment in FIG. 9 provides the amino acid sequences of ANNOT 543117 (SEQ ID NO:161), ANNOT 1464138 (SEQ ID NO:164), CLONE 481263 (SEQ ID NO:167), GI 50929499 (SEQ ID NO:168), CLONE 1806767 (SEQ ID NO:222), CLONE 378258 (SEQ ID NO:246), GI 90657540 (SEQ ID NO:283), and GI 92894700 (SEQ ID NO:285). Other homologs and/or orthologs include Public GI no. 20198186 (SEQ ID NO:162), Ceres GDNA ANNOT ID no. 1512068 (SEQ ID NO:166), and Public GI no. 50726629 (SEQ ID NO:169).

In some cases, a protein-modulating polypeptide includes a polypeptide having at least 80% sequence identity, e.g., 80%, 83%, 85%, 86%, 87%, 88%, 89%, 90%, 91%, 92%, 93%, 94%, 95%, 96%, 97%, 98%, or 99% sequence identity, to an amino acid sequence corresponding to SEQ ID NO:162, SEQ ID NO:164, SEQ ID NO:166, SEQ ID NO:167, SEQ ID NO:168, SEQ ID NO:169, SEQ ID NO:222, SEQ ID NO:246, SEQ ID NO:283, or SEQ ID NO:285.

A protein-modulating polypeptide can have a Ribosomal_L36 domain characteristic of a ribosomal protein L36. About ⅔ of the mass of a ribosome consists of RNA and ⅓ consists of protein. The proteins are named according to the subunit of the ribosome to which they belong. Small ribosomal subunits are designated S1 to S31, while large ribosomal subunits are designated L1 to L44. Many ribosomal proteins, particularly those of the large subunit, are composed of a globular, surface-exposed domain with long finger-like projections that extend into the rRNA core to stabilize its structure. Most of the proteins interact with multiple RNA elements, often from different domains. SEQ ID NO:175 sets forth the amino acid sequence of an Arabidopsis clone, identified herein as Ceres ANNOT ID no. 570373 (SEQ ID NO:174), that is predicted to encode a polypeptide containing a Ribosomal_L36 domain.

A protein-modulating polypeptide can comprise the amino acid sequence set forth in SEQ ID NO:175. Alternatively, a protein-modulating polypeptide can be a homolog, ortholog, or variant of the polypeptide having the amino acid sequence set forth in SEQ ID NO:175. For example, a protein-modulating polypeptide can have an amino acid sequence with at least 45% sequence identity, e.g., 46%, 50%, 55%, 60%, 65%, 70%, 75%, 80%, 85%, 90%, 95%, 97%, 98%, or 99% sequence identity, to the amino acid sequence set forth in SEQ ID NO:175.

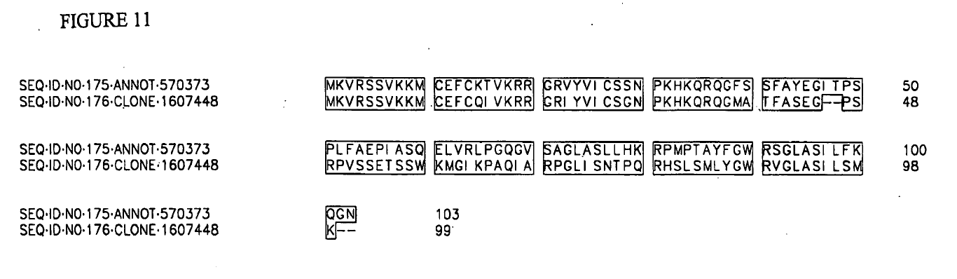

Amino acid sequences of homologs and/or orthologs of the polypeptide having the amino acid sequence set forth in SEQ ID NO:175 are provided in FIG. 11. The alignment in FIG. 11 provides the amino acid sequences of ANNOT 570373 (SEQ ID NO:175) and CLONE 1607448 (SEQ ID NO:176). Other homologs and/or orthologs include Ceres CLONE ID no. 1043684 (SEQ ID NO:177) and Ceres CLONE ID no. 723341 (SEQ ID NO:178).

In some cases, a protein-modulating polypeptide includes a polypeptide having at least 80% sequence identity, e.g., 80%, 83%, 85%, 86%, 87%, 88%, 89%, 90%, 91%, 92%, 93%, 94%, 95%, 96%, 97%, 98%, or 99% sequence identity, to an amino acid sequence corresponding to SEQ ID NO:176, SEQ ID NO:177, or SEQ ID NO:178.

A protein-modulating polypeptide can have an RNA recognition motif. RNA recognition motifs, also known as RRM, RBD, or RNP domains, are found in a variety of RNA binding polypeptides, including heterogeneous nuclear ribonucleoproteins (hnRNPs), polypeptides implicated in regulation of alternative splicing, and polypeptide components of small nuclear ribonucleoproteins (snRNPs). The RRM motif also appears in a few single stranded DNA binding proteins. The RRM structure consists of four strands and two helices arranged in an alpha/beta sandwich, with a third helix present during RNA binding in some cases. SEQ ID NO:180 sets forth the amino acid sequence of an Arabidopsis clone, identified herein as Ceres CLONE ID no. 4595 (SEQ ID NO:179), that is predicted to encode a polypeptide containing an RNA recognition motif.

A protein-modulating polypeptide can comprise the amino acid sequence set forth in SEQ ID NO:180. Alternatively, a protein-modulating polypeptide can be a homolog, ortholog, or variant of the polypeptide having the amino acid sequence set forth in SEQ ID NO:180. For example, a protein-modulating polypeptide can have an amino acid sequence with at least 40% sequence identity, e.g., 41%, 45%, 50%, 55%, 60%, 65%, 70%, 75%, 80%, 85%, 90%, 95%, 97%, 98%, or 99% sequence identity, to the amino acid sequence set forth in SEQ ID NO:180.

A protein-modulating polypeptide can have a Glyco_hydro_{—}28 domain characteristic of a glycosyl hydrolase family 28 polypeptide. Glycosyl hydrolases hydrolyze the glycosidic bond between two or more carbohydrates, or between a carbohydrate and a non-carbohydrate moiety. Glycoside hydrolase family 28 comprises enzymes with several known activities, including polygalacturonase, exo-polygalacturonase, and rhamnogalacturonase. The fold of glycosyl hydrolase polypeptides is better conserved than the sequence of glycosyl hydrolase polypeptides. SEQ ID NO:191 sets forth the amino acid sequence of a Glycine max clone, identified herein as Ceres CLONE ID no. 558363 (SEQ ID NO:190), that is predicted to encode a polypeptide containing a Glyco_hydro_{—}28 domain.

A protein-modulating polypeptide can comprise the amino acid sequence set forth in SEQ ID NO:191. Alternatively, a protein-modulating polypeptide can be a homolog, ortholog, or variant of the polypeptide having the amino acid sequence set forth in SEQ ID NO:191. For example, a protein-modulating polypeptide can have an amino acid sequence with at least 45% sequence identity, e.g., 46%, 50%, 55%, 60%, 65%, 70%, 75%, 80%, 85%, 90%, 95%, 97%, 98%, or 99% sequence identity, to the amino acid sequence set forth in SEQ ID NO:191.

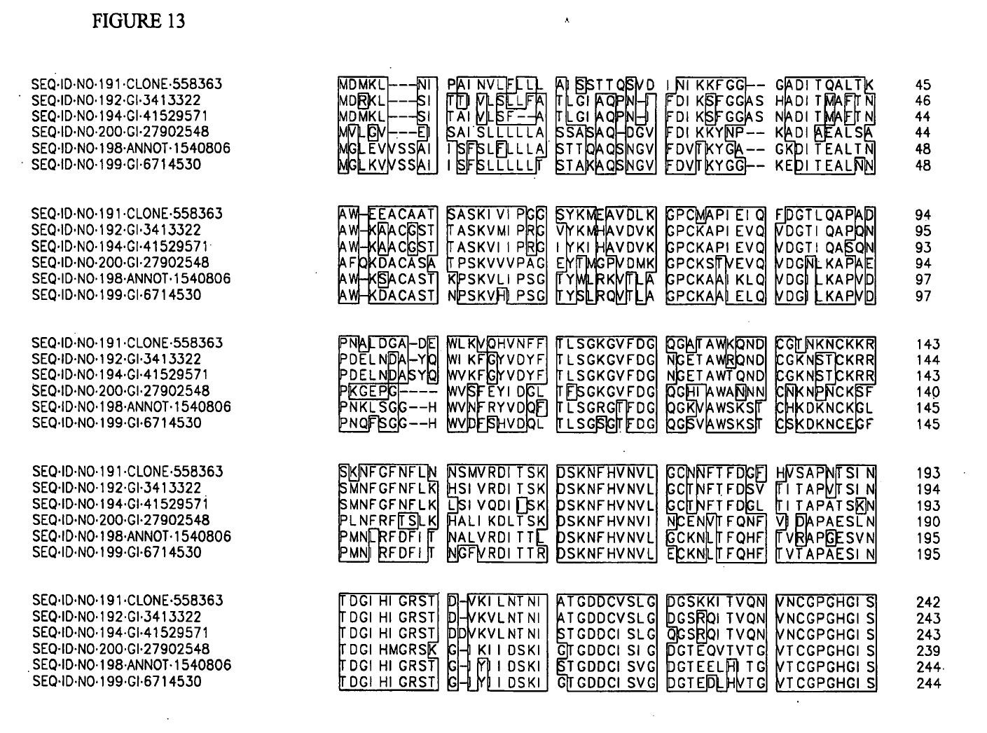

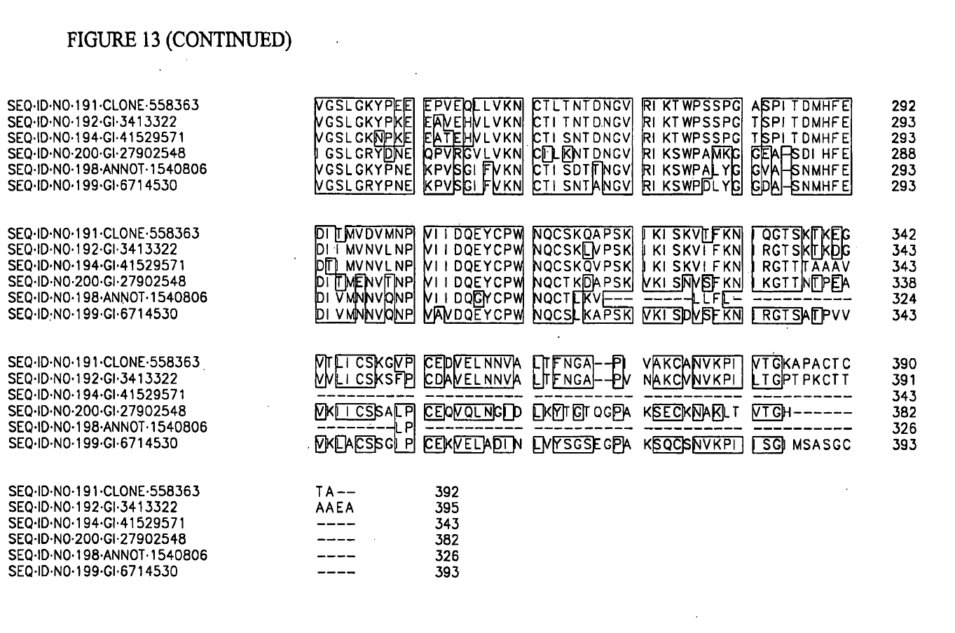

Amino acid sequences of homologs and/or orthologs of the polypeptide having the amino acid sequence set forth in SEQ ID NO:191 are provided in FIG. 13. The alignment in FIG. 13 provides the amino acid sequences of CLONE 558363 (SEQ ID NO:191), GI 3413322 (SEQ ID NO:192), GI 41529571 (SEQ ID NO:194), ANNOT 1540806 (SEQ ID NO:198), GI 6714530 (SEQ ID NO:199), and GI 27902548 (SEQ ID NO:200). Other homologs and/or orthologs include Ceres CLONE ID no. 522929 (SEQ ID NO:193), Public GI no. 29123382 (SEQ ID NO:195), Public GI no. 668998 (SEQ ID NO:196), Public GI no. 6714526 (SEQ ID NO:201), Public GI no. 6714524 (SEQ ID NO:202), and Public GI no. 6714528 (SEQ ID NO:203).

In some cases, a protein-modulating polypeptide includes a polypeptide having at least 80% sequence identity, e.g., 80%, 83%, 85%, 86%, 87%, 88%, 89%, 90%, 91%, 92%, 93%, 94%, 95%, 96%, 97%, 98%, or 99% sequence identity, to an amino acid sequence corresponding to SEQ ID NO:192, SEQ ID NO:193, SEQ ID NO:194, SEQ ID NO:195, SEQ ID NO:196, SEQ ID NO:198, SEQ ID NO:199, SEQ ID NO:200, SEQ ID NO:201, SEQ ID NO:202, or SEQ ID NO:203.

SEQ ID NO:80, SEQ ID NO:102, SEQ ID NO:119, SEQ ID NO:171, SEQ ID NO:182, SEQ ID NO:205, and SEQ ID NO:209 set forth the amino acid sequences of DNA clones, identified herein as Ceres CLONE ID no. 33780 (SEQ ID NO:79), Ceres CLONE ID no. 42577 (SEQ ID NO:101), Ceres CLONE ID no. 400568 (SEQ ID NO:118), Ceres ANNOT ID no. 546661 (SEQ ID NO:170), Ceres CLONE ID no. 531679 (SEQ ID NO:181), Ceres CLONE ID no. 8161 (SEQ ID NO:204), and Ceres cDNA ID no. 36509475 (SEQ ID NO:208), respectively, each of which is predicted to encode a polypeptide that does not have homology to an existing protein family based on Pfam analysis.

A protein-modulating polypeptide can comprise the amino acid sequence set forth in SEQ ID NO:80, SEQ ID NO:102, SEQ ID NO:119, SEQ ID NO:171, SEQ ID NO:182, SEQ ID NO:205, or SEQ ID NO:209. Alternatively, a protein-modulating polypeptide can be a homolog, ortholog, or variant of the polypeptide having the amino acid sequence set forth in SEQ ID NO:80, SEQ ID NO:102, SEQ ID NO:119, SEQ ID NO:171, SEQ ID NO:182, SEQ ID NO:205, or SEQ ID NO:209. For example, a protein-modulating polypeptide can have an amino acid sequence with at least 40% sequence identity, e.g., 40%, 45%, 50%, 55%, 60%, 65%, 70%, 75%, 80%, 85%, 90%, 95%, 97%, 98%, or 99% sequence identity, to the amino acid sequence set forth in SEQ ID NO:80, SEQ ID NO:102, SEQ ID NO:119, SEQ ID NO:171, SEQ ID NO:182, SEQ ID NO:205, or SEQ ID NO:209.

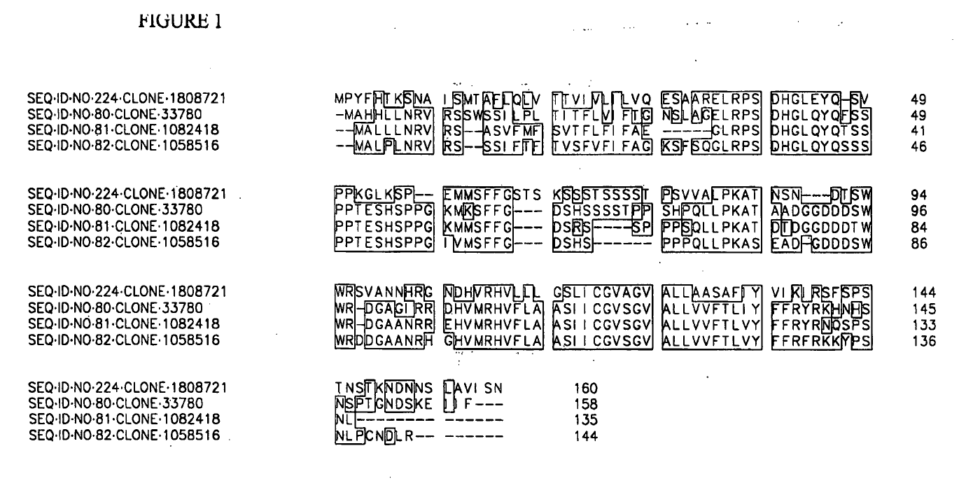

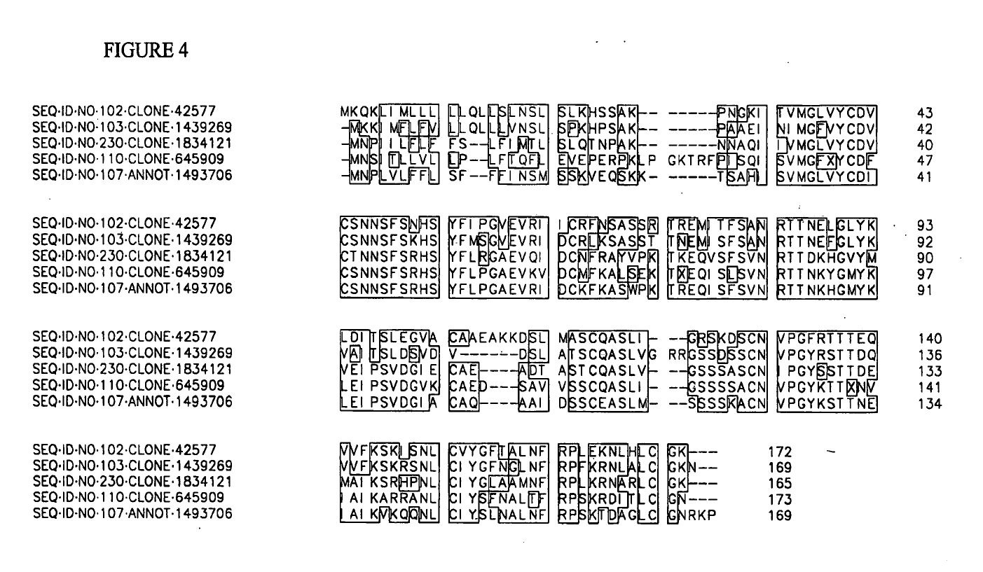

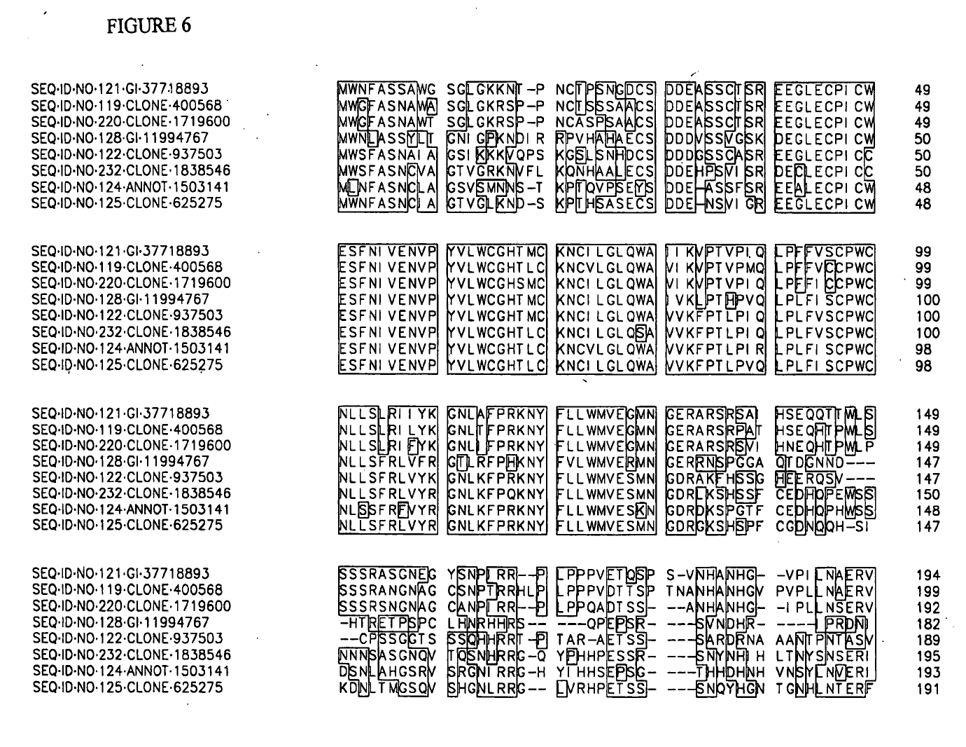

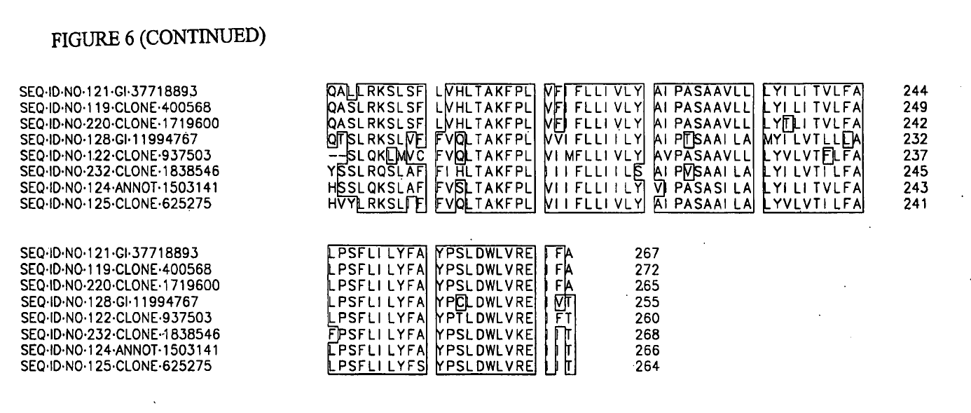

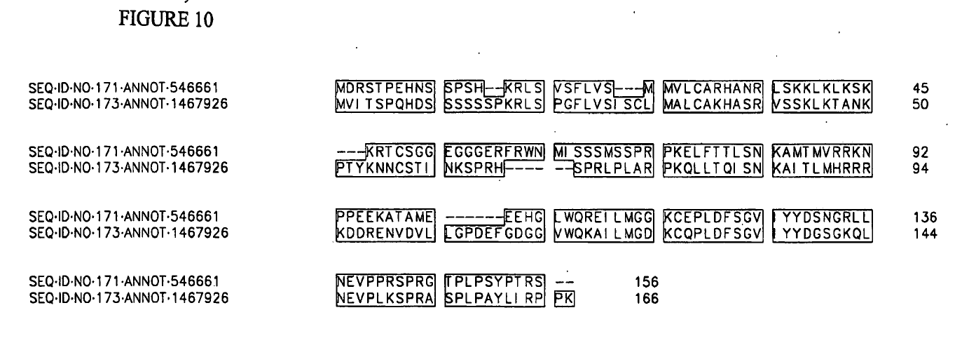

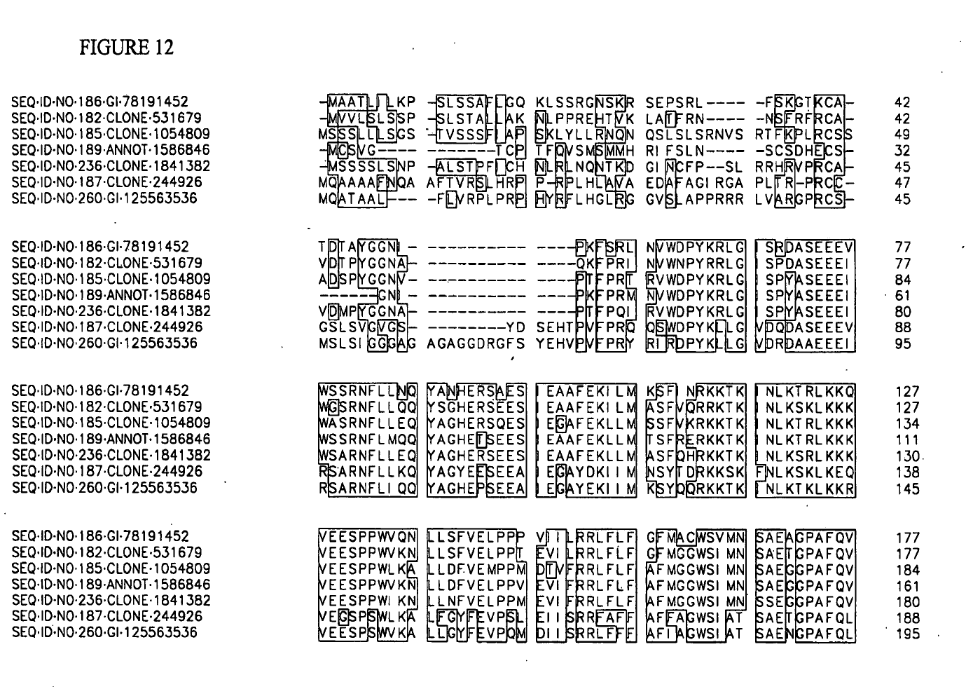

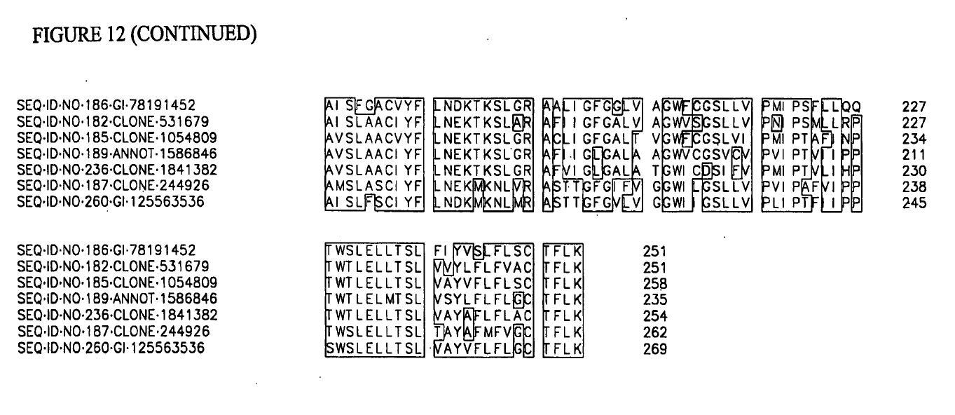

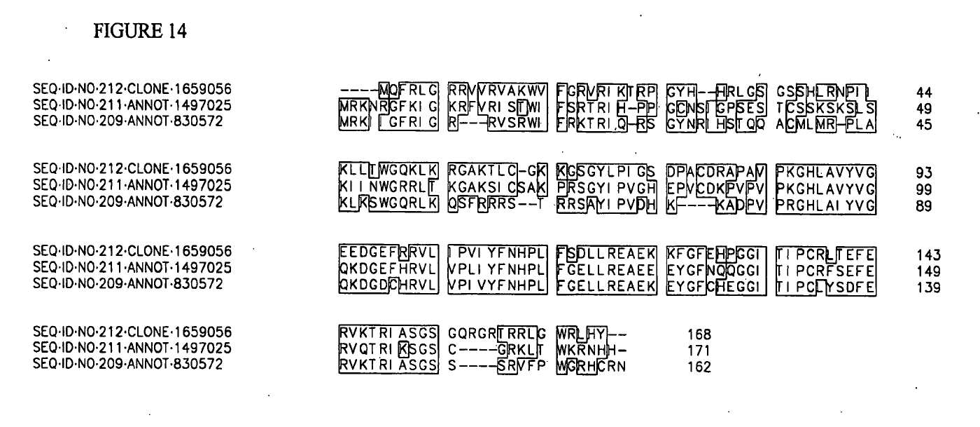

Amino acid sequences of homologs and/or orthologs of the polypeptide having the amino acid sequence set forth in SEQ ID NO:80, SEQ ID NO:102, SEQ ID NO:119, SEQ ID NO:171, SEQ ID NO:182, and SEQ ID NO:209 are provided in FIG. 1, FIG. 4, FIG. 6, FIG. 10, FIG. 12, and FIG. 14, respectively.

The alignment in FIG. 1 provides the amino acid sequences of CLONE 33780 (SEQ ID NO:80), CLONE 1082418 (SEQ ID NO:81), CLONE 1058516 (SEQ ID NO:82), and CLONE 1808721 (SEQ ID NO:224).

The alignment in FIG. 4 provides the amino acid sequences of CLONE 42577 (SEQ ID NO:102), CLONE 1439269 (SEQ ID NO:103), ANNOT 1493706 (SEQ ID NO:107), CLONE 645909 (SEQ ID NO:110), and CLONE 1834121 (SEQ ID NO:230). Other homologs and/or orthologs include Ceres ANNOT ID no. 1440825 (SEQ ID NO:105), Ceres ANNOT ID no. 1485758 (SEQ ID NO:109), and Ceres CLONE ID no. 1838785 (SEQ ID NO:234).

The alignment in FIG. 6 provides the amino acid sequences of CLONE 400568 (SEQ ID NO:119), GI 37718893 (SEQ ID NO:121), CLONE 937503 (SEQ ID NO:122), ANNOT 1503141 (SEQ ID NO:124), CLONE 625275 (SEQ ID NO:125), GI 11994767 (SEQ ID NO:128), CLONE 1719600 (SEQ ID NO:220), and CLONE 1838546 (SEQ ID NO:232). Other homologs and/or orthologs include Ceres CLONE ID no. 1549251 (SEQ ID NO:120), Ceres CLONE ID no. 1371622 (SEQ ID NO:126), Ceres CLONE ID no. 511038 (SEQ ID NO:127), Ceres CLONE ID no. 1845447 (SEQ ID NO:238), and Ceres CLONE ID no. 1935338 (SEQ ID NO:242).

The alignment in FIG. 10 provides the amino acid sequences of ANNOT 546661 (SEQ ID NO:171) and ANNOT 1467926 (SEQ ID NO:173).

The alignment in FIG. 12 provides the amino acid sequences of CLONE 531679 (SEQ ID NO:182), CLONE 1054809 (SEQ ID NO:185), GI 78191452 (SEQ ID NO:186), CLONE 244926 (SEQ ID NO:187), ANNOT 1586846 (SEQ ID NO:189), CLONE 1841382 (SEQ ID NO:236), and GI 125563536 (SEQ ID NO:260). Other homologs and/or orthologs include Ceres CLONE ID no. 100141 (SEQ ID NO:184).

The alignment in FIG. 14 provides the amino acid sequences of ANNOT 830572 (SEQ ID NO:209), ANNOT 1497025 (SEQ ID NO:211), and CLONE 1659056 (SEQ ID NO:212).

In some cases, a protein-modulating polypeptide includes a polypeptide having at least 80% sequence identity, e.g., 80%, 83%, 85%, 86%, 87%, 88%, 89%, 90%, 91%, 92%, 93%, 94%, 95%, 96%, 97%, 98%, or 99% sequence identity, to an amino acid sequence corresponding to SEQ ID NO:81, SEQ ID NO:82, SEQ ID NO:103, SEQ ID NO:105, SEQ ID NO:107, SEQ ID NO:109, SEQ ID NO:110, SEQ ID NO:120, SEQ ID NO:121, SEQ ID NO:122, SEQ ID NO:124, SEQ ID NO:125, SEQ ID NO:126, SEQ ID NO:127, SEQ ID NO:128, SEQ ID NO:173, SEQ ID NO:184, SEQ ID NO:185, SEQ ID NO:186, SEQ ID NO:187, SEQ ID NO:189, SEQ ID NO:211, SEQ ID NO:212, SEQ ID NO:220, SEQ ID NO:224, SEQ ID NO:230, SEQ ID NO:232, SEQ ID NO:234, SEQ ID NO:236, SEQ ID NO:238, SEQ ID NO:242, or SEQ ID NO:260.

A protein-modulating polypeptide encoded by a recombinant nucleic acid can be a native protein-modulating polypeptide, i.e., one or more additional copies of the coding sequence for a protein-modulating polypeptide that is naturally present in the cell. Alternatively, a protein-modulating polypeptide can be heterologous to the cell, e.g., a transgenic Lycopersicon plant can contain the coding sequence for a kinase polypeptide from a Glycine plant.

A protein-modulating polypeptide can include additional amino acids that are not involved in protein modulation, and thus can be longer than would otherwise be the case. For example, a protein-modulating polypeptide can include an amino acid sequence that functions as a reporter. Such a protein-modulating polypeptide can be a fusion protein in which a green fluorescent protein (GFP) polypeptide is fused to, e.g., SEQ ID NO:102, or in which a yellow fluorescent protein (YFP) polypeptide is fused to, e.g., SEQ ID NO:141. In some embodiments, a protein-modulating polypeptide includes a purification tag, a chloroplast transit peptide, a mitochondrial transit peptide, or a leader sequence added to the amino or carboxy terminus.

Protein-modulating polypeptide candidates suitable for use in the invention can be identified by analysis of nucleotide and polypeptide sequence alignments. For example, performing a query on a database of nucleotide or polypeptide sequences can identify homologs and/or orthologs of protein-modulating polypeptides. Sequence analysis can involve BLAST, Reciprocal BLAST, or PSI-BLAST analysis of nonredundant databases using known protein-modulating polypeptide amino acid sequences. Those polypeptides in the database that have greater than 40% sequence identity can be identified as candidates for further evaluation for suitability as a protein-modulating polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains suspected of being present in protein-modulating polypeptides, e.g., conserved functional domains.

The identification of conserved regions in a template or subject polypeptide can facilitate production of variants of wild type protein-modulating polypeptides. Conserved regions can be identified by locating a region within the primary amino acid sequence of a template polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains at sanger.ac.uk/Pfam and genome.wustl.edu/Pfam. A description of the information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999). Amino acid residues corresponding to Pfam domains included in protein-modulating polypeptides provided herein are set forth in the sequence listing. For example, amino acid residues 93 to 356 of the amino acid sequence set forth in SEQ ID NO:84 correspond to a polyprenyl_synt domain, as indicated in fields <222> and <223> for SEQ ID NO:84 in the sequence listing.

Conserved regions also can be determined by aligning sequences of the same or related polypeptides from closely related species. Closely related species preferably are from the same family. In some embodiments, alignment of sequences from two different species is adequate. For example, sequences from Arabidopsis and Zea mays can be used to identify one or more conserved regions.

Typically, polypeptides that exhibit at least about 40% amino acid sequence identity are useful to identify conserved regions. Conserved regions of related polypeptides can exhibit at least 45% amino acid sequence identity (e.g., at least 50%, at least 60%, at least 70%, at least 80%, or at least 90% amino acid sequence identity). In some embodiments, a conserved region of target and template polypeptides exhibit at least 92%, 94%, 96%, 98%, or 99% amino acid sequence identity. Amino acid sequence identity can be deduced from amino acid or nucleotide sequences. In certain cases, highly conserved domains have been identified within protein-modulating polypeptides. These conserved regions can be useful in identifying functionally similar (orthologous) protein-modulating polypeptides.

In some instances, suitable protein-modulating polypeptides can be synthesized on the basis of consensus functional domains and/or conserved regions in polypeptides that are homologous protein-modulating polypeptides. Domains are groups of substantially contiguous amino acids in a polypeptide that can be used to characterize protein families and/or parts of proteins. Such domains have a “fingerprint” or “signature” that can comprise conserved (1) primary sequence, (2) secondary structure, and/or (3) three-dimensional conformation. Generally, domains are correlated with specific in vitro and/or in vivo activities. A domain can have a length of from 10 amino acids to 400 amino acids, e.g., 10 to 50 amino acids, or 25 to 100 amino acids, or 35 to 65 amino acids, or 35 to 55 amino acids, or 45 to 60 amino acids, or 200 to 300 amino acids, or 300 to 400 amino acids.

Representative homologs and/or orthologs of protein-modulating polypeptides are shown in FIGS. 1-18. Each Figure represents an alignment of the amino acid sequence of a protein-modulating polypeptide with the amino acid sequences of corresponding homologs and/or orthologs. Amino acid sequences of protein-modulating polypeptides and their corresponding homologs and/or orthologs have been aligned to identify conserved amino acids, as shown in FIGS. 1-18. A dash in an aligned sequence represents a gap, i.e., a lack of an amino acid at that position. Identical amino acids or conserved amino acid substitutions among aligned sequences are identified by boxes. Each conserved region contains a sequence of contiguous amino acid residues.

Useful polypeptides can be constructed based on the conserved regions in FIG. 1, FIG. 2, FIG. 3, FIG. 4, FIG. 5, FIG. 6, FIG. 7, FIG. 8, FIG. 9, FIG. 10, FIG. 11, FIG. 12, FIG. 13, FIG. 14, FIG. 15, FIG. 16, FIG. 17, or FIG. 18. Such a polypeptide includes the conserved regions arranged in the order depicted in the Figure from amino-terminal end to carboxyterminal end. Such a polypeptide may also include zero, one, or more than one amino acid in positions marked by dashes. When no amino acids are present at positions marked by dashes, the length of such a polypeptide is the sum of the amino acid residues in all conserved regions. When amino acids are present at all positions marked by dashes, such a polypeptide has a length that is the sum of the amino acid residues in all conserved regions and all dashes.

Conserved regions can be identified by homologous polypeptide sequence analysis as described above. The suitability of polypeptides for use as protein-modulating polypeptides can be evaluated by functional complementation studies.

Useful polypeptides can also be identified based on the polypeptides set forth in any of FIGS. 1-18 using algorithms designated as Hidden Markov Models. A “Hidden Markov Model (HMM)” is a statistical model of a consensus sequence for a group of homologous and/or orthologous polypeptides. See, Durbin et al., Biological Sequence Analysis Probabilistic Models of proteins and Nucleic Acids, Cambridge University Press, Cambridge, UK (1998). An HMM is generated by the program HMMER 2.3.2 using the multiple sequence alignment of the group of homologous and/or orthologous sequences as input and the default program parameters. The multiple sequence alignment is generated by ProbCons (Do et al., Genome Res., 15(2):330-40 (2005)) version 1.11 using a set of default parameters: -c, —consistency REPS of 2; -ir, —iterative-refinement REPS of 100; -pre, —pre-training REPS of 0. ProbCons is a public domain software program provided by Stanford University.

The default parameters for building an HMM (hmmbuild) are as follows: the default “architecture prior” (archpri) used by MAP architecture construction is 0.85, and the default cutoff threshold (idlevel) used to determine the effective sequence number is 0.62. The HMMER 2.3.2 package was released Oct. 3, 2003 under a GNU general public license, and is available from various sources on the World Wide Web such as hmmerjanelia.org, hmmer.wustl.edu, and fr.com/hmmer232/. Hmmbuild outputs the model as a text file.

The HMM for a group of homologous and/or orthologous polypeptides can be used to determine the likelihood that a subject polypeptide sequence is a better fit to that particular HMM than to a null HMM generated using a group of sequences that are not homologous and/or orthologous. The likelihood that a subject polypeptide sequence is a better fit to an HMM than to a null HMM is indicated by the HMM bit score, a number generated when the subject sequence is fitted to the HMM profile using the HMMER hmmsearch program. The following default parameters are used when running hmmsearch: the default E-value cutoff (E) is 10.0, the default bit score cutoff (T) is negative infinity, the default number of sequences in a database (Z) is the real number of sequences in the database, the default E-value cutoff for the per-domain ranked hit list (domE) is infinity, and the default bit score cutoff for the per-domain ranked hit list (domT) is negative infinity. A high HMM bit score indicates a greater likelihood that the subject sequence carries out one or more of the biochemical or physiological function(s) of the polypeptides used to generate the HMM. A high HMM bit score is at least 20, and often is higher.

A protein-modulating polypeptide can fit an HMM provided herein with an HMM bit score greater than 20 (e.g., greater than 30, 40, 50, 60, 70, 80, 90, 100, 200, 300, 400, or 500). In some cases, a protein-modulating polypeptide can fit an HMM provided herein with an HMM bit score that is about 50%, 60%, 70%, 80%, 90%, or 95% of the HMM bit score of any homologous and/or orthologous polypeptide provided in any of Tables 29-46. In some cases, a protein-modulating polypeptide can fit an HMM described herein with an HMM bit score greater than 20, and can have a conserved domain, e.g., a PFAM domain, or a conserved region having 70% or greater sequence identity (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity) to a conserved domain or region present in a protein-modulating polypeptide disclosed herein.

For example, a protein-modulating polypeptide can fit an HMM generated using the amino acid sequences set forth in FIG. 1 with an HMM bit score that is greater than about 150 (e.g., greater than about 160, 170, 180, 190, 200, 225, 250, 275, 300, 325, 350, 375, or 400). In some cases, a protein-modulating polypeptide can fit an HMM generated using the amino acid sequences set forth in FIG. 2 with an HMM bit score that is greater than about 300 (e.g., greater than about 325, 350, 375, 400, 425, 450, 475, 500, 525, 550, 575, 600, 625, 650, 675, 700, 725, 750, 775, or 800). In some cases, a protein-modulating polypeptide can fit an HMM generated using the amino acid sequences set forth in FIG. 3 with an HMM bit score that is greater than about 300 (e.g., greater than about 350, 400, 450, 500, 550, 575, 600, 650, 700, 750, 800, 850, 900, 950, 1000, 1050, 1100, 1150, or 1200). In some cases, a protein-modulating polypeptide can fit an HMM generated using the amino acid sequences set forth in FIG. 4 with an HMM bit score that is greater than about 150 (e.g., greater than about 175, 200, 225, 250, 275, 300, 325, 350, 375, or 400). In some cases, a protein-modulating polypeptide can fit an HMM generated using the amino acid sequences set forth in FIG. 5 with an HMM bit score that is greater than about 400 (e.g., greater than about 450, 500, 550, 600, 650, 700, 750, 800, 850, 900, 950, or 1000). In some cases, a protein-modulating polypeptide can fit an HMM generated using the amino acid sequences set forth in FIG. 6 with an HMM bit score that is greater than about 150 (e.g., greater than about 175, 200, 225, 250, 275, 300, 325, 350, 400, 425, 450, 475, 500, 525, 550, 575, or 600). In some cases, a protein-modulating polypeptide can fit an HMM generated using the amino acid sequences set forth in FIG. 7 with an HMM bit score that is greater than about 250 (e.g., greater than about 275, 300, 350, 400, 450, 500, 550, 600, 650, 700, or 725). In some cases, a protein-modulating polypeptide can fit an HMM generated using the amino acid sequences set forth in FIG. 8 with an HMM bit score that is greater than about 100 (e.g., greater than about 125, 150, 175, 200, 225, 250, 275, 300, 325, 350, 375, 400, or 425). In some cases, a protein-modulating polypeptide can fit an HMM generated using the amino acid sequences set forth in FIG. 9 with an HMM bit score that is greater than about 500 (e.g., greater than about 525, 550, 600, 650, 700, 750, 800, 850, 900, 950, 1000, 1050, 1100, 1150, 1200, 1250, 1300, 1350, 1400, or 1425). In some cases, a protein-modulating polypeptide can fit an HMM generated using the amino acid sequences set forth in FIG. 10 with an HMM bit score that is greater than about 175 (e.g., greater than about 200, 225, 250, 275, 300, 325, 350, 375, 400, 425, 450, or 475). In some cases, a protein-modulating polypeptide can fit an HMM generated using the amino acid sequences set forth in FIG. 11 with an HMM bit score that is greater than about 100 (e.g., greater than about 125, 150, 175, 200, 225, 250, 275, or 300). In some cases, a protein-modulating polypeptide can fit an HMM generated using the amino acid sequences set forth in FIG. 12 with an HMM bit score that is greater than about 250 (e.g., greater than about 275, 300, 325, 350, 375, 400, 425, 450, 475, 500, 525, 550, 575, 600, 625, or 650). In some cases, a protein-modulating polypeptide can fit an HMM generated using the amino acid sequences set forth in FIG. 13 with an HMM bit score that is greater than about 350 (e.g., greater than about 375, 400, 450, 500, 550, 600, 650, 700, 750, 800, 850, 900, 950, or 1000). In some cases, a protein-modulating polypeptide can fit an HMM generated using the amino acid sequences set forth in FIG. 14 with an HMM bit score that is greater than about 200 (e.g., greater than about 225, 250, 275, 300, 325, 350, 375, 400, 425, 450, 475, or 500). In some cases, a protein-modulating polypeptide can fit an HMM generated using the amino acid sequences set forth in FIG. 15 with an HMM bit score that is greater than about 600 (e.g., greater than about 650, 700, 750, 800, 850, 900, 950, 1000, 1050, 1100, 1150, 1200, 1250, 1300, 1350, 1400, or 1450). In some cases, a protein-modulating polypeptide can fit an HMM generated using the amino acid sequences set forth in FIG. 16 with an HMM bit score that is greater than about 200 (e.g., greater than about 225, 250, 275, 300, 325, 350, 375, 400, 425, 450, 475, 500, 525, 550, 575, or 600). In some cases, a protein-modulating polypeptide can fit an HMM generated using the amino acid sequences set forth in FIG. 17 with an HMM bit score that is greater than about 450 (e.g., greater than about 475, 500, 550, 600, 650, 700, 750, 800, 850, 900, 950, 1000, 1100, 1200, 1300, 1400, 1500, or 1600). In some cases, a protein-modulating polypeptide can fit an HMM generated using the amino acid sequences set forth in FIG. 18 with an HMM bit score that is greater than about 250 (e.g., greater than about 300, 350, 400, 450, 500, 550, 600, 650, 700, 750, 800, 850, or 900).

### Nucleic Acids

The terms “nucleic acid” and “polynucleotide” are used interchangeably herein, and refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, plasmids, vectors, isolated DNA of any sequence, isolated. RNA of any sequence, nucleic acid probes, and primers, as well as nucleic acid analogs.

Nucleic acids described herein include protein-modulating nucleic acids. Protein-modulating nucleic acids can be effective to modulate protein levels when transcribed in a plant or plant cell. SEQ ID NO:206 sets forth the nucleotide sequence of a DNA clone identified herein as Ceres cDNA ID no. 23698270. A protein-modulating nucleic acid can comprise the nucleotide sequence set forth in SEQ ID NO:206. Alternatively, a protein-modulating nucleic acid can be a variant of the nucleic acid having the nucleotide sequence set forth in SEQ ID NO:206. For example, a protein-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:206.

An “isolated” nucleic acid can be, for example, a naturally-occurring DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus, or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

Isolated nucleic acid molecules can be produced by standard techniques. For example, polymerase chain reaction (PCR) techniques can be used to obtain an isolated nucleic acid containing a nucleotide sequence described herein. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring DNA.

As used herein, the term “percent sequence identity” refers to the degree of identity between any given query sequence and a subject sequence. A subject sequence typically has a length that is more than 80 percent, e.g., more than 82, 85, 87, 89, 90, 93, 95, 97, 99, 100, 105, 110, 115, or 120 percent, of the length of the query sequence. A query nucleic acid or amino acid sequence is aligned to one or more subject nucleic acid or amino acid sequences using the computer program ClustalW (version 1.83, default parameters), which allows alignments of nucleic acid or protein sequences to be carried out across their entire length (global alignment). Chema et al., Nucleic Acids Res., 31(13):3497-500 (2003).

ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The output is a sequence alignment that reflects the relationship between sequences. ClustalW can be run, for example, at the Baylor College of Medicine Search Launcher site (searchlauncher.bcm.tmc.edu/multi-align/multi-align.html) and at the European Bioinformatics Institute site on the World Wide Web (ebi.ac.uk/clustalw).

To determine a percent identity between a query sequence and a subject sequence, ClustalW divides the number of identities in the best alignment by the number of residues compared (gap positions are excluded), and multiplies the result by 100. The output is the percent identity of the subject sequence with respect to the query sequence. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

The term “exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. It will be appreciated that an exogenous nucleic acid may have been introduced into a progenitor and not into the cell under consideration. For example, a transgenic plant containing an exogenous nucleic acid can be the progeny of a cross between a stably transformed plant and a non-transgenic plant. Such progeny are considered to contain the exogenous nucleic acid.

Recombinant constructs are also provided herein and can be used to transform plants or plant cells in order to modulate protein levels. A recombinant nucleic acid construct can comprise a nucleic acid encoding a protein-modulating polypeptide as described herein, operably linked to a regulatory region suitable for expressing the protein-modulating polypeptide in the plant or cell. Thus, a nucleic acid can comprise a coding sequence that encodes any of the protein-modulating polypeptides as set forth in SEQ ID NOs:80-82, SEQ ID NOs:84-93, SEQ ID NOs:95-96, SEQ ID NOs:98-100, SEQ ID NOs:102-103, SEQ ID NO:105, SEQ ID NO:107, SEQ ID NOs:109-110, SEQ ID NO:112, SEQ ID NO:114, SEQ ID NOs:116-117, SEQ ID NOs:119-122, SEQ ID NOs:124-128, SEQ ID NO:130, SEQ ID NOs:132-133, SEQ ID NOs:135-139, SEQ ID NOs:141-150, SEQ ID NO:152, SEQ ID NOs:154-155, SEQ ID NOs:157-159, SEQ ID NOs:161-162, SEQ ID NO:164, SEQ ID NOs:166-169, SEQ ID NO:171, SEQ ID NO:173, SEQ ID NOs:175-178, SEQ ID NO:180, SEQ ID NOs:182-187, SEQ ID NO:189, SEQ ID NOs:191-196, SEQ ID NOs:198-203, SEQ ID NO:205, SEQ ID NO:209, SEQ ID NOs:211-212, SEQ ID NOs:214-215, SEQ ID NOs:217-218, SEQ ID NO:220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NOs:248-250, SEQ ID NO:252, SEQ ID NO:254, SEQ ID NOs:256-286, SEQ ID NO:315, SEQ ID NOs:317-328, SEQ ID NOs:330, SEQ ID NO:332, SEQ ID NO:334, SEQ ID NOs:336-337, SEQ ID NO:339, SEQ ID NO:341, or SEQ ID NOs:343-349.

Examples of nucleic acids encoding protein-modulating polypeptides are set forth in SEQ ID NO:79, SEQ ID NO:83, SEQ ID NO:94, SEQ ID NO:97, SEQ ID NO:101, SEQ ID NO:104, SEQ ID NO:106, SEQ ID NO:108, SEQ ID NO:111, SEQ ID NO:113, SEQ ID NO:115, SEQ ID NO:118, SEQ ID NO:123, SEQ ID NO:129, SEQ ID NO:131, SEQ ID NO:134, SEQ ID NO:140, SEQ ID NO:151, SEQ ID NO:153, SEQ ID NO:156, SEQ ID NO:160, SEQ ID NO:163, SEQ ID NO:165, SEQ ID NO:170, SEQ ID NO:172, SEQ ID NO:174, SEQ ID NO:179, SEQ ID NO:181, SEQ ID NO:188, SEQ ID NO:190, SEQ ID NO:197, SEQ ID NO:204, SEQ ID NO:206, SEQ ID NO:208, SEQ ID NO:210, SEQ ID NO:213, SEQ ID NO:216, SEQ ID NO:219, SEQ ID NO:221, SEQ ID NO:223, SEQ ID NO:225, SEQ ID NO:227, SEQ ID NO:229, SEQ ID NO:231, SEQ ID NO:233, SEQ ID NO:235, SEQ ID NO:237, SEQ ID NO:239, SEQ ID NO:241, SEQ ID NO:243, SEQ ID NO:245, SEQ ID NO:247, SEQ ID NO:251, SEQ ID NO:253, SEQ ID NO:255, SEQ ID NOs:287-314, SEQ ID NO:316, SEQ ID NO:329, SEQ ID NO:331, SEQ ID NO:333, SEQ ID NO:335, SEQ ID NO:338, SEQ ID NO:340, and SEQ ID NO:342.

In some cases, a recombinant nucleic acid construct can include a nucleic acid comprising less than the full-length of a coding sequence. For example, a recombinant nucleic acid construct can comprise a protein-modulating nucleic acid having the nucleotide sequence set forth in SEQ ID NO:206. Typically, such a construct also includes a regulatory region operably linked to the protein-modulating nucleic acid.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given protein-modulating polypeptide can be modified such that optimal expression in a particular plant species is obtained, using appropriate codon bias tables for that species.

Vectors containing nucleic acids such as those described herein also are provided A “vector” is a replicon, such as a plasmid, phage, or cosmid, into which another DNA segment may be inserted so as to bring about the replication of the inserted segment.

Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes a regulatory region. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The vectors provided herein also can include, for example, origins of replication, scaffold attachment regions (SARs), and/or markers. A marker gene can confer a selectable phenotype on a plant cell. For example, a marker can confer biocide resistance, such as resistance to an antibiotic (e.g., kanamycin, G418, bleomycin, or hygromycin), or an herbicide (e.g., chlorosulfuron or phosphinothricin). In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

### Regulatory Regions

The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

As used herein, the term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site, or about 2,000 nucleotides upstream of the transcription start site. A promoter typically comprises at least a core (basal) promoter. A promoter also may include at least one control element, such as an enhancer sequence, an upstream element or an upstream activation region (UAR). For example, a suitable enhancer is a cis-regulatory element (−212 to −154) from the upstream region of the octopine synthase (ocs) gene. Fromm et al., The Plant Cell, 1:977-984 (1989). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Some suitable promoters initiate transcription only, or predominantly, in certain cell types. For example, a promoter that is active predominantly in a reproductive tissue (e.g., fruit, ovule, pollen, pistils, female gametophyte, egg cell, central cell, nucellus, suspensor, synergid cell, flowers, embryonic tissue, embryo sac, embryo, zygote, endosperm, integument, or seed coat) can be used. Thus, as used herein a cell type- or tissue-preferential promoter is one that drives expression preferentially in the target tissue, but may also lead to some expression in other cell types or tissues as well. Methods for identifying and characterizing promoter regions in plant genomic DNA include, for example, those described in the following references: Jordano et al., Plant Cell, 1:855-866 (1989); Bustos et al., Plant Cell, 1:839-854 (1989); Green et al., EMBO J., 7:4035-4044 (1988); Meier et al., Plant Cell, 3:309-316 (1991); and Zhang et al., Plant Physiology, 110:1069-1079 (1996).

Examples of various classes of promoters are described below. Some of the promoters indicated below as well as additional promoters are described in more detail in U.S. Patent Application Ser. Nos. 60/505,689; 60/518,075; 60/544,771; 60/558,869; 60/583,691; 60/619,181; 60/637,140; 60/757,544; 60/776,307; 10/957,569; 11/058,689; 11/172,703; 11/208,308; 11/274,890; 60/583,609; 60/612,891; 11/097,589; 11/233,726; 10/950,321; PCT/US05/011105; PCT/US05/034308; and PCT/US05/23639. Nucleotide sequences of promoters are set forth in SEQ ID NOs:1-78. It will be appreciated that a promoter may meet criteria for one classification based on its activity in one plant species, and yet meet criteria for a different classification based on its activity in another plant species.

### Broadly Expressing Promoters

A promoter can be said to be “broadly expressing” when it promotes transcription in many, but not necessarily all, plant tissues. For example, a broadly expressing promoter can promote transcription of an operably linked sequence in one or more of the shoot, shoot tip (apex), and leaves, but weakly or not at all in tissues such as roots or stems. As another example, a broadly expressing promoter can promote transcription of an operably linked sequence in one or more of the stem, shoot, shoot tip (apex), and leaves, but can promote transcription weakly or not at all in tissues such as reproductive tissues of flowers and developing seeds. Non-limiting examples of broadly expressing promoters that can be included in the nucleic acid constructs provided herein include the p326 (SEQ ID NO:76), YP0144 (SEQ ID NO:55), YP0190 (SEQ ID NO:59), p13879 (SEQ ID NO:75), YP0050 (SEQ ID NO:35), p32449 (SEQ ID NO:77), 21876 (SEQ ID NO:1), YP0158 (SEQ ID NO:57), YP0214 (SEQ ID NO:61), YP0380 (SEQ ID NO:70), PT0848 (SEQ ID NO:26), and PT0633 (SEQ ID NO:7) promoters. Additional examples include the cauliflower mosaic virus (CaMV) 35S promoter, the mannopine synthase (MAS) promoter, the 1′ or 2′ promoters derived from T-DNA of Agrobacterium tumefaciens, the figwort mosaic virus 34S promoter, actin promoters such as the rice actin promoter, and ubiquitin promoters such as the maize ubiquitin-1 promoter. In some cases, the CaMV 35S promoter is excluded from the category of broadly expressing promoters.

Root Promoters

Root-active promoters confer transcription in root tissue, e.g., root endodermis, root epidermis, or root vascular tissues. In some embodiments, root-active promoters are root-preferential promoters, i.e., confer transcription only or predominantly in root tissue. Root-preferential promoters include the YP0128 (SEQ ID NO:52), YP0275 (SEQ ID NO:63), PT0625 (SEQ ID NO:6), PT0660 (SEQ ID NO:9), PT0683 (SEQ ID NO:14), and PT0758 (SEQ ID NO:22) promoters. Other root-preferential promoters include the PT0613 (SEQ ID NO:5), PT0672 (SEQ ID NO:111), PT0688 (SEQ ID NO:15), and PT0837 (SEQ ID NO:24) promoters, which drive transcription primarily in root tissue and to a lesser extent in ovules and/or seeds. Other examples of root-preferential promoters include the root-specific subdomains of the CaMV 35S promoter (Lam et al., Proc. Natl. Acad. Sci. USA, 86:7890-7894 (1989)), root cell specific promoters reported by Conkling et al., Plant Physiol., 93:1203-1211 (1990), and the tobacco RD2 promoter.

Maturing Endosperm Promoters

In some embodiments, promoters that drive transcription in maturing endosperm can be useful. Transcription from a maturing endosperm promoter typically begins after fertilization and occurs primarily in endosperm tissue during seed development and is typically highest during the cellularization phase. Most suitable are promoters that are active predominantly in maturing endosperm, although promoters that are also active in other tissues can sometimes be used. Non-limiting examples of maturing endosperm promoters that can be included in the nucleic acid constructs provided herein include the napin promoter, the Arcelin-5 promoter, the phaseolin promoter (Bustos et al., Plant Cell, 1(9):839-853 (1989)), the soybean trypsin inhibitor promoter (Riggs et al., Plant Cell, 1(6):609-621 (1989)), the ACP promoter (Baerson et al., Plant Mol. Biol., 22(2):255-267 (1993)), the stearoyl-ACP desaturase promoter (Slocombe et al., Plant Physiol., 104(4):167-176 (1994)), the soybean α subunit of β-conglycinin promoter (Chen et al., Proc. Natl. Acad. Sci. USA, 83:8560-8564 (1986)), the oleosin promoter (Hong et al., Plant Mol. Biol., 34(3):549-555 (1997)), and zein promoters, such as the 15 kD zein promoter, the 16 kD zein promoter, 19 kD zein promoter, 22 kD zein promoter and 27 kD zein promoter. Also suitable are the Osgt-1 promoter from the rice glutelin-1 gene (Zheng et al., Mol. Cell Biol., 13:5829-5842 (1993)), the beta-amylase promoter, and the barley hordein promoter. Other maturing endosperm promoters include the YP0092 (SEQ ID NO:38), PT0676 (SEQ ID NO:12), and PT0708 (SEQ ID NO:17) promoters.

Ovary Tissue Promoters

Promoters that are active in ovary tissues such as the ovule wall and mesocarp can also be useful, e.g., a polygalacturonidase promoter, the banana TRX promoter, and the melon actin promoter. Examples of promoters that are active primarily in ovules include YP0007 (SEQ ID NO:30), YP0111 (SEQ ID NO:46), YP0092 (SEQ ID NO:38), YP0103 (SEQ ID NO:43), YP0028 (SEQ ID NO:33), YP0121 (SEQ ID NO:51), YP0008 (SEQ ID NO:31), YP0039 (SEQ ID NO:34), YP0115 (SEQ ID NO:47), YP0119 (SEQ ID NO:49), YP0120 (SEQ ID NO:50), and YP0374 (SEQ ID NO:68).

Embryo Sac/Early Endosperm Promoters

To achieve expression in embryo sac/early endosperm, regulatory regions can be used that are active in polar nuclei and/or the central cell, or in precursors to polar nuclei, but not in egg cells or precursors to egg cells. Most suitable are promoters that drive expression only or predominantly in polar nuclei or precursors thereto and/or the central cell. A pattern of transcription that extends from polar nuclei into early endosperm development can also be found with embryo sac/early endosperm-preferential promoters, although transcription typically decreases significantly in later endosperm development during and after the cellularization phase. Expression in the zygote or developing embryo typically is not present with embryo sac/early endosperm promoters.

Promoters that may be suitable include those derived from the following genes: Arabidopsis viviparous-1 (see, GenBank® No. U93215); Arabidopsis atmycl (see, Urao (1996) Plant Mol. Biol., 32:571-57; Conceicao (1994) Plant, 5:493-505); Arabidopsis FIE (GenBank: No. AF129516); Arabidopsis MEA; Arabidopsis FIS2 (GenBank No. AF096096); and FIE 1.1 (U.S. Pat. No. 6,906,244). Other promoters that may be suitable include those derived from the following genes: maize MAC1 (see, Sheridan (1996) Genetics, 142:1009-1020); maize Cat3 (see, GenBank No. L05934; Abler (1993) Plant Mol. Biol., 22:10131-1038). Other promoters include the following Arabidopsis promoters: YP0039 (SEQ ID NO:34), YP0101 (SEQ ID NO:41), YP0102 (SEQ ID NO:42), YP0110 (SEQ ID NO:45), YP0117 (SEQ ID NO:48), YP0119 (SEQ ID NO:49), YP0137 (SEQ ID NO:53), DME, YP0285 (SEQ ID NO:64), and YP0212 (SEQ ID NO:60). Other promoters that may be useful include the following rice promoters: p530c10, pOsFIE2-2, pOsMEA, pOsYp102, and pOsYp285.

Embryo Promoters

Regulatory regions that preferentially drive transcription in zygotic cells following fertilization can provide embryo-preferential expression. Most suitable are promoters that preferentially drive transcription in early stage embryos prior to the heart stage, but expression in late stage and maturing embryos is also suitable. Embryo-preferential promoters include the barley lipid transfer protein (Ltpl) promoter (Plant Cell Rep (2001) 20:647-654), YP0097 (SEQ ID NO:40), YP0107 (SEQ ID NO:44), YP0088 (SEQ ID NO:37), YP0143 (SEQ ID NO:54), YP0156 (SEQ ID NO:56), PT0650 (SEQ ID NO:8), PT0695 (SEQ ID NO:16), PT0723 (SEQ ID NO:19), PT0838 (SEQ ID NO:25), PT0879 (SEQ ID NO:28), and PT0740 (SEQ ID NO:20).

Photosynthetic Tissue Promoters

Promoters active in photosynthetic tissue confer transcription in green tissues such as leaves and stems. Most suitable are promoters that drive expression only or predominantly in such tissues. Examples of such promoters include the ribulose-1,5-bisphosphate carboxylase (RbcS) promoters such as the RbcS promoter from eastern larch (Larix laricina), the pine cab6 promoter (Yamamoto et al., Plant Cell Physiol., 35:773-778 (1994)), the Cab-1 promoter from wheat (Fejes et al., Plant Mol. Biol., 15:921-932 (1990)), the CAB-1 promoter from spinach (Lubberstedt et al., Plant Physiol., 104:997-1006 (1994)), the cab1R promoter from rice (Luan et al., Plant Cell, 4:971-981 (1992)), the pyruvate orthophosphate dikinase (PPDK) promoter from corn (Matsuoka et al., Proc. Natl. Acad. Sci. USA, 90:9586-9590 (1993)), the tobacco Lhcb1*2 promoter (Cerdan et al., Plant Mol. Biol., 33:245-255 (1997)), the Arabidopsis thaliana SUC2 sucrose-H+ symporter promoter (Truemit et al., Planta, 196:564-570 (1995)), and thylakoid membrane protein promoters from spinach (psaD, psaF, psaE, PC, FNR, atpC, atpD, cab, rbcS). Other photosynthetic tissue promoters include PT0535 (SEQ ID NO:3), PT0668 (SEQ ID NO:2), PT0886 (SEQ ID NO:29), YP0144 (SEQ ID NO:55), YP0380 (SEQ ID NO:70), and PT0585 (SEQ ID NO:4).

Vascular Tissue Promoters

Examples of promoters that have high or preferential activity in vascular bundles include YP0087, YP0093, YP0108, YP0022, and YP0080. Other vascular tissue-preferential promoters include the glycine-rich cell wall protein GRP 1.8 promoter (Keller and Baumgartner, Plant Cell, 3(10):1051-1061 (1991)), the Commelina yellow mottle virus (CoYMV) promoter (Medberry et al., Plant Cell, 4(2):185-192 (1992)), and the rice tungro bacilliform virus (RTBV) promoter (Dai et al., Proc. Natl. Acad. Sci. USA, 101(2):687-692 (2004)).

Inducible Promoters

Inducible promoters confer transcription in response to external stimuli such as chemical agents or environmental stimuli. For example, inducible promoters can confer transcription in response to hormones such as giberellic acid or ethylene, or in response to light or drought. Examples of drought-inducible promoters include YP0380 (SEQ ID NO:70), PT0848 (SEQ ID NO:26), YP0381 (SEQ ID NO:71), YP0337 (SEQ ID NO:66), PT0633 (SEQ ID NO:7), YP0374 (SEQ ID NO:68), PT0710 (SEQ ID NO:18), YP0356 (SEQ ID NO:67), YP0385 (SEQ ID NO:73), YP0396 (SEQ ID NO:74), YP0388, YP0384 (SEQ ID NO:72), PT0688 (SEQ ID NO:15), YP0286 (SEQ ID NO:65), YP0377 (SEQ ID NO:69), PD1367 (SEQ ID NO:78), PDO901, and PD0898. Nitrogen-inducible promoters include PT0863 (SEQ ID NO:27), PT0829 (SEQ ID NO:23), PT0665 (SEQ ID NO:10), and PT0886 (SEQ ID NO:29).

Basal Promoters

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

Other Promoters

Other classes of promoters include, but are not limited to, leaf-preferential, stem/shoot-preferential, callus-preferential, guard cell-preferential such as PT0678 (SEQ ID NO:13), and senescence-preferential promoters. Promoters designated YP0086 (SEQ ID NO:36), YP0188 (SEQ ID NO:58), YP0263 (SEQ ID NO:62), PT0758 (SEQ ID NO:22), PT0743 (SEQ ID NO:21), PT0829 (SEQ ID NO:23), YP0119 (SEQ ID NO:49), and YP0096 (SEQ ID NO:39), as described in the above-referenced patent applications, may also be useful.

Other Regulatory Regions

A 5′ untranslated region (UTR) can be included in nucleic acid constructs described herein. A 5′ UTR is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences, e.g., a nopaline synthase termination sequence.

It will be understood that more than one regulatory region may be present in a recombinant polynucleotide, e.g., introns, enhancers, upstream activation regions, transcription terminators, and inducible elements. Thus, more than one regulatory region can be operably linked to the sequence of a polynucleotide encoding a protein-modulating polypeptide.

Regulatory regions, such as promoters for endogenous genes, can be obtained by chemical synthesis or by subcloning from a genomic DNA that includes such a regulatory region. A nucleic acid comprising such a regulatory region can also include flanking sequences that contain restriction enzyme sites that facilitate subsequent manipulation.

### Transgenic Plants and Plant Cells

The invention also features transgenic plant cells and plants comprising at least one recombinant nucleic acid construct described herein. A plant or plant cell can be transformed by having a construct integrated into its genome, i.e., can be stably transformed. Stably transformed cells typically retain the introduced nucleic acid with each cell division. A plant or plant cell can also be transiently transformed such that the construct is not integrated into its genome. Transiently transformed cells typically lose all or some portion of the introduced nucleic acid construct with each cell division such that the introduced nucleic acid cannot be detected in daughter cells after a sufficient number of cell divisions. Both transiently transformed and stably transformed transgenic plants and plant cells can be useful in the methods described herein.

Transgenic plant cells used in methods described herein can constitute part or all of a whole plant. Such plants can be grown in a manner suitable for the species under consideration, either in a growth chamber, a greenhouse, or in a field. Transgenic plants can be bred as desired for a particular purpose, e.g., to introduce a recombinant nucleic acid into other lines, to transfer a recombinant nucleic acid to other species, or for further selection of other desirable traits. Alternatively, transgenic plants can be propagated vegetatively for those species amenable to such techniques. As used herein, a transgenic plant also refers to progeny of an initial transgenic plant. Progeny includes descendants of a particular plant or plant line. Progeny of an instant plant include seeds formed on F₁, F₂, F₃, F₄, F₅, F₆ and subsequent generation plants, or seeds formed on BC₁, BC₂, BC₃, and subsequent generation plants, or seeds formed on F₁BC₁, F₁BC₂, F₁BC₃, and subsequent generation plants. The designation F₁ refers to the progeny of a cross between two parents that are genetically distinct. The designations F₂, F₃, F₄, F₅ and F₆ refer to subsequent generations of self- or sib-pollinated progeny of an F₁ plant. Seeds produced by a transgenic plant can be grown and then selfed (or outcrossed and selfed) to obtain seeds homozygous for the nucleic acid construct.

Transgenic plants can be grown in suspension culture, or tissue or organ culture. For the purposes of this invention, solid and/or liquid tissue culture techniques can be used. When using solid medium, transgenic plant cells can be placed directly onto the medium or can be placed onto a filter that is then placed in contact with the medium. When using liquid medium, transgenic plant cells can be placed onto a flotation device, e.g., a porous membrane that contacts the liquid medium. Solid medium typically is made from liquid medium by adding agar. For example, a solid medium can be Murashige and Skoog (MS) medium containing agar and a suitable concentration of an auxin, e.g., 2,4-dichlorophenoxyacetic acid (2,4-D), and a suitable concentration of a cytokinin, e.g., kinetin.

When transiently transformed plant cells are used, a reporter sequence encoding a reporter polypeptide having a reporter activity can be included in the transformation procedure and an assay for reporter activity or expression can be performed at a suitable time after transformation. A suitable time for conducting the assay typically is about 1-21 days after transformation, e.g., about 1-14 days, about 1-7 days, or about 1-3 days. The use of transient assays is particularly convenient for rapid analysis in different species, or to confirm expression of a heterologous protein-modulating polypeptide whose expression has not previously been confirmed in particular recipient cells.

Techniques for introducing nucleic acids into monocotyledonous and dicotyledonous plants are known in the art, and include, without limitation, Agrobacterium-mediated transformation, viral vector-mediated transformation, electroporation and particle gun transformation, e.g., U.S. Pat. Nos. 5,538,880; 5,204,253; 6,329,571 and 6,013,863. If a cell or cultured tissue is used as the recipient tissue for transformation, plants can be regenerated from transformed cultures if desired, by techniques known to those skilled in the art.

### Plant Species

The polynucleotides and vectors described herein can be used to transform a number of monocotyledonous and dicotyledonous plants and plant cell systems. Suitable species include Panicum spp., Sorghum spp., Miscanthus spp., Saccharum spp., Erianthus spp., Populus spp., Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Medicago sativa (alfalfa), Arundo donax (giant reed), Secale cereale (rye), Salix spp. (willow), Eucalyptus spp. (eucalyptus), Triticale (wheat X rye) and bamboo.

Suitable species also include Panicum virgatum (switchgrass), Sorghum bicolor (sorghum), Miscanthus giganteus (miscanthus), Saccharum sp. (energycane), Populus balsamifera (poplar), Helianthus annuus (sunflower), Carthamus tinctorius (safflower), Jatropha curcas (jatropha), Ricinus communis (castor), Elaeis guineensis (palm), Linum usitatissimum (flax), Brassica juncea, Beta vulgaris (sugarbeet), Manihot esculenta (cassaya), Lycopersicon esculentum (tomato), Lactuca sativa (lettuce), Musa paradisiaca (banana), Solanum tuberosum (potato), Brassica oleracea (broccoli, cauliflower, brusselsprouts), Camellia sinensis (tea), Fragaria ananassa (strawberry), Theobroma cacao (cocoa), Coffea arabica (coffee), Vitis vinifera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solanum melongena (eggplant), Parthenium argentatum (guayule), Hevea spp. (rubber), Mentha spicata (mint), Mentha piperita (mint), Bixa orellana, Alstroemeria spp., Nicotiana tabacum (tobacco), Uniola paniculata (oats), bentgrass (Agrostis spp.), Populus tremuloides (aspen), Pinus spp. (pine), Abies spp. (fir), and Acer spp. (maple).

Thus, the methods and compositions described herein can be used with dicotyledonous plants belonging, for example, to the orders Apiales, Arecales, Aristochiales, Asterales, Batales, Campanulales, Capparales, Caryophyllales, Casuarinales, Celastrales, Cornales, Cucurbitales, Diapensales, Dilleniales, Dipsacales, Ebenales, Ericales, Eucomiales, Euphorbiales, Fabales, Fagales, Gentianales, Geraniales, Haloragales, Hamamelidales, Illiciales, Juglandales, Lamiales, Laurales, Lecythidales, Leitneriales, Linales, Magniolales, Malvales, Myricales, Myrtales, Nymphaeales, Papaverales, Piperales, Plantaginales, Plumbaginales, Podostemales, Polemoniales, Polygalales, Polygonales, Populus, Primulales, Proteales, Rafflesiales, Ranunculales, Rhamnales, Rosales, Rubiales, Salicales, Santales, Sapindales, Sarraceniaceae, Scrophulariales, Solanales, Trochodendrales, Theales, Umbellales, Urticales, and Violales. The methods and compositions described herein also can be utilized with monocotyledonous plants such as those belonging to the orders Alismatales, Arales, Arecales, Asparagales, Bromeliales, Commelinales, Cyclanthales, Cyperales, Eriocaulales, Hydrocharitales, Juncales, Liliales, Najadales, Orchidales, Pandanales, Poales, Restionales, Triuridales, Typhales, Zingiberales, and with plants belonging to Gymnospermae, e.g., Cycadales, Ginkgoales, Gnetales, and Pinales.

The methods and compositions can be used over a broad range of plant species, including species from the dicot genera Brassica, Carthamus, Glycine, Gossypium, Helianthus, Jatropha, Lupinus, Parthenium, Populus, and Ricinus; and the monocot genera Elaeis, Festuca, Hordeum, Lolium, Oryza, Panicum, Pennisetum, Phleum, Poa, Saccharum, Secale, Sorghum, Triticosecale, Triticum, and Zea. In some embodiments, a plant is a member of the species Panicum virgatum (switchgrass), Sorghum bicolor (sorghum), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza saliva (rice), Helianthus annuus (sunflower), Medicago saliva (alfalfa), Beta vulgaris (sugarbeet), Pennisetum glaucum (pearl millet), or Lupinus albus (lupin).

### Methods of Inhibiting Expression of Protein-Modulating Polypeptides

The polynucleotides and recombinant vectors described herein can be used to express or inhibit expression of a protein-modulating polypeptide in a plant species of interest. The term “expression” refers to the process of converting genetic information of a polynucleotide into RNA through transcription, which is catalyzed by an enzyme, RNA polymerase, and into protein, through translation of mRNA on ribosomes. “Up-regulation” or “activation” refers to regulation that increases the production of expression products (mRNA, polypeptide, or both) relative to basal or native states, while “down-regulation” or “repression” refers to regulation that decreases production of expression products (mRNA, polypeptide, or both) relative to basal or native states.

A number of nucleic-acid based methods, including antisense RNA, co-suppression, ribozyme directed RNA cleavage, and RNA interference (RNAi) can be used to inhibit protein expression in plants. Antisense technology is one well-known method. In this method, a nucleic acid segment from a gene to be repressed is cloned and operably linked to a promoter so that the antisense strand of RNA is transcribed. The recombinant vector is then transformed into plants, as described above, and the antisense strand of RNA is produced. The nucleic acid segment need not be the entire sequence of the gene to be repressed, but typically will be substantially complementary to at least a portion of the sense strand of the gene to be repressed. Generally, higher homology can be used to compensate for the use of a shorter sequence. Typically, a sequence of at least 30 nucleotides is used, e.g., at least 40, 50, 80, 100, 200, 500 nucleotides or more.

Thus, for example, an isolated nucleic acid provided herein can be an antisense nucleic acid to any of the aforementioned nucleic acids encoding a protein-modulating polypeptide set forth in SEQ ID NOs:80-82, SEQ ID NOs:84-93, SEQ ID NOs:95-96, SEQ ID NOs:98-100, SEQ ID NOs:102-103, SEQ ID NO:105, SEQ ID NO:107, SEQ ID NOs:109-110, SEQ ID NO:112, SEQ ID NO:114, SEQ ID NOs:116-117, SEQ ID NOs:119-122, SEQ ID NOs:124-128, SEQ ID NO:130, SEQ ID NOs:132-133, SEQ ID NOs:135-139, SEQ ID NOs:141-150, SEQ ID NO:152, SEQ ID NOs:154-155, SEQ ID NOs:157-159, SEQ ID NOs:161-162, SEQ ID NO:164, SEQ ID NOs:166-169, SEQ ID NO:171, SEQ ID NO:173, SEQ ID NOs:175-178, SEQ ID NO:180, SEQ ID NOs:182-187, SEQ ID NO:189, SEQ ID NOs:191-196, SEQ ID NOs:198-203, SEQ ID NO:205, SEQ ID NO:209, SEQ ID NOs:211-212, SEQ ID NOs:214-215, SEQ ID NOs:217-218, SEQ ID NO:220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NOs:248-250, SEQ ID NO:252, SEQ ID NO:254, SEQ ID NOs:256-286, SEQ ID NO:315, SEQ ID NOs:317-328, SEQ ID NOs:330, SEQ ID NO:332, SEQ ID NO:334, SEQ ID NOs:336-337, SEQ ID NO:339, SEQ ID NO:341, or SEQ ID NOs:343-349. A nucleic acid that decreases the level of a transcription or translation product of a gene encoding a protein-modulating polypeptide is transcribed into an antisense nucleic acid that anneals to the sense coding sequence of the protein-modulating polypeptide.

Constructs containing operably linked nucleic acid molecules in the sense orientation can also be used to inhibit the expression of a gene. The transcription product can be similar or identical to the sense coding sequence of a protein-modulating polypeptide. The transcription product can also be unpolyadenylated, lack a 5′ cap structure, or contain an unsplicable intron. Methods of co-suppression using a full-length cDNA as well as a partial cDNA sequence are known in the art. See, e.g., U.S. Pat. No. 5,231,020.

In another method, a nucleic acid can be transcribed into a ribozyme, or catalytic RNA, that affects expression of an mRNA. (See, U.S. Pat. No. 6,423,885). Ribozymes can be designed to specifically pair with virtually any target RNA and cleave the phosphodiester backbone at a specific location, thereby functionally inactivating the target RNA. Heterologous nucleic acids can encode ribozymes designed to cleave particular mRNA transcripts, thus preventing expression of a polypeptide. Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contain a 5′-UG-3′ nucleotide sequence. The construction and production of hammerhead ribozymes is known in the art. See, for example, U.S. Pat. No. 5,254,678 and WO 02/46449 and references cited therein. Hammerhead ribozyme sequences can be embedded in a stable RNA such as a transfer RNA (tRNA) to increase cleavage efficiency in vivo. Perriman et al., Proc. Natl. Acad. Sci. USA, 92(13):6175-6179 (1995); de Feyter and Gaudron, Methods in Molecular Biology, Vol. 74, Chapter 43, “Expressing Ribozymes in Plants,” Edited by Turner, P. C., Humana Press Inc., Totowa, N.J. RNA endoribonucleases which have been described, such as the one that occurs naturally in Tetrahymena thermophila, can be useful. See, for example, U.S. Pat. Nos. 4,987,071 and 6,423,885.

RNAi can also be used to inhibit the expression of a gene. For example, a construct can be prepared that includes a sequence that is transcribed into an interfering RNA. Such an RNA can be one that can anneal to itself, e.g., a double stranded RNA having a stem-loop structure. One strand of the stem portion of a double stranded RNA comprises a sequence that is similar or identical to the sense coding sequence of the polypeptide of interest, and that is from about 10 nucleotides to about 2,500 nucleotides in length. The length of the sequence that is similar or identical to the sense coding sequence can be from 10 nucleotides to 500 nucleotides, from 15 nucleotides to 300 nucleotides, from 20 nucleotides to 100 nucleotides, or from 25 nucleotides to 100 nucleotides. The other strand of the stem portion of a double stranded RNA comprises a sequence that is similar or identical to the antisense strand of the coding sequence of the polypeptide of interest, and can have a length that is shorter, the same as, or longer than the corresponding length of the sense sequence. The loop portion of a double stranded RNA can be from 10 nucleotides to 5,000 nucleotides, e.g., from 15 nucleotides to 1,000 nucleotides, from 20 nucleotides to 500 nucleotides, or from 25 nucleotides to 200 nucleotides. The loop portion of the RNA can include an intron. A construct including a sequence that is transcribed into an interfering RNA is transformed into plants as described above. Methods for using RNAi to inhibit the expression of a gene are known to those of skill in the art. See, e.g., U.S. Pat. Nos. 5,034,323; 6,326,527; 6,452,067; 6,573,099; 6,753,139; and 6,777,588. See also WO 97/01952; WO 98/53083; WO 99/32619; WO 98/36083; and U.S. Patent Publications 20030175965, 20030175783, 20040214330, and 20030180945.

In some nucleic-acid based methods for inhibition of gene expression in plants, a suitable nucleic acid can be a nucleic acid analog. Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-deoxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered morpholino ring, or peptide nucleic acids, in which the deoxyphosphate backbone is replaced by a pseudopeptide backbone and the four bases are retained. See, for example, Summerton and Weller, 1997, Antisense Nucleic Acid Drug Dev., 7:187-195; Hyrup et al., Bioorgan. Med. Chem., 4:5-23 (1996). In addition, the deoxyphosphate backbone can be replaced with, for example, a phosphorothioate or phosphorodithioate backbone, a phosphoroamidite, or an alkyl phosphotriester backbone.

### Transgenic Plant Phenotypes

A transformed cell, callus, tissue, or plant can be identified and isolated by selecting or screening the engineered plant material for particular traits or activities, e.g., expression of a selectable marker gene or modulation of protein content. Such screening and selection methodologies are well known to those having ordinary skill in the art. In addition, physical and biochemical methods can be used to identify transformants. These include Southern analysis or PCR amplification for detection of a polynucleotide; Northern blots, S1 RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are well known.

A population of transgenic plants can be screened and/or selected for those members of the population that have a desired trait or phenotype conferred by expression of the transgene. Selection and/or screening can be carried out over one or more generations, which can be useful to identify those plants that have a desired trait, such as a modulated level of protein. Selection and/or screening can also be carried out in more than one geographic location. In some cases, transgenic plants can be grown and selected under conditions which induce a desired phenotype or are otherwise necessary to produce a desired phenotype in a transgenic plant. In addition, selection and/or screening can be carried out during a particular developmental stage in which the phenotype is exhibited by the plant.

The phenotype of a transgenic plant can be evaluated relative to a control plant that does not express the exogenous polynucleotide of interest, such as a corresponding wild type plant, a corresponding plant that is not transgenic for the exogenous polynucleotide of interest but otherwise is of the same genetic background as the transgenic plant of interest, or a corresponding plant of the same genetic background in which expression of the polypeptide is suppressed, inhibited, or not induced (e.g., where expression is under the control of an inducible promoter). A plant can be said “not to express” a polypeptide when the plant exhibits less than 10%, e.g., less than 9%, 8%, 7%, 6%, 5%, 4%, 3%, 2%, 1%, 0.5%, 0.1%, 0.01%, or 0.001%, of the amount of polypeptide or mRNA encoding the polypeptide exhibited by the plant of interest. Expression can be evaluated using methods including, for example, RT-PCR, Northern blots, S1 RNase protection, primer extensions, Western blots, protein gel electrophoresis, immunoprecipitation, enzyme-linked immunoassays, chip assays, and mass spectrometry. It should be noted that if a polypeptide is expressed under the control of a tissue-preferential or broadly expressing promoter, expression can be evaluated in the entire plant or in a selected tissue. Similarly, if a polypeptide is expressed at a particular time, e.g., at a particular time in development or upon induction, expression can be evaluated selectively at a desired time period.

In some embodiments, a plant in which expression of a protein-modulating polypeptide is modulated can have increased levels of seed protein. For example, a protein-modulating polypeptide described herein can be expressed in a transgenic plant, resulting in increased levels of seed protein. The seed protein level can be increased by at least 2 percent, e.g., 2, 3, 4, 5, 6, 7, 8, 9, 10, 11, 12, 13, 14, 15, 16, 17, 18, 19, 20, 25, 30, 35, 40, 45, or more than 45 percent, as compared to the seed protein level in a corresponding control plant that does not express the transgene. In some embodiments, a plant in which expression of a protein-modulating polypeptide is modulated can have decreased levels of seed protein. The seed protein level can be decreased by at least 2 percent, e.g., 2, 3, 4, 5, 10, 15, 20, 25, 30, 35, or more than 35 percent, as compared to the seed protein level in a corresponding control plant that does not express the transgene.

Plants for which modulation of levels of seed protein can be useful include, without limitation, amaranth, barley, beans, canola, coffee, cotton, edible nuts (e.g., almond, brazil nut, cashew, hazelnut, macadamia nut, peanut, pecan, pine nut, pistachio, walnut), field corn, millet, oat, oil palm, peas, popcorn, rapeseed, rice, rye, safflower, sorghum, soybean, sunflower, sweet corn, and wheat. Increases in seed protein in such plants can provide improved nutritional content in geographic locales where dietary intake of protein/amino acid is often insufficient. Decreases in seed protein in such plants can be useful in situations where seeds are not the primary plant part that is harvested for human or animal consumption.

In some embodiments, a plant in which expression of a protein-modulating polypeptide is modulated can have increased or decreased levels of protein in one or more non-seed tissues, e.g., leaf tissues, stem tissues, root or corm tissues, or fruit tissues other than seed. For example, the protein level can be increased by at least 2 percent, e.g., 2, 3, 4, 5, 6, 7, 8, 9, 10, 11, 12, 13, 14, 15, 16, 17, 18, 19, 20, 25, 30, 35, 40, 45, or more than 45 percent, as compared to the protein level in a corresponding control plant that does not express the transgene. In some embodiments, a plant in which expression of a protein-modulating polypeptide is modulated can have decreased levels of protein in one or more non-seed tissues. The protein level can be decreased by at least 2 percent, e.g., 2, 3, 4, 5, 10, 15, 20, 25, 30, 35, or more than 35 percent, as compared to the protein level in a corresponding control plant that does not express the transgene.

Plants for which modulation of levels of protein in non-seed tissues can be useful include, without limitation, alfalfa, amaranth, apple, banana, barley, beans, bluegrass, broccoli, carrot, cherry, clover, coffee, fescue, field corn, grape, grapefruit, lemon, lettuce, mango, melon, millet, oat, oil palm, onion, orange, peach, peanut, pear, peas, pineapple, plum, popcorn, potato, rapeseed, rice, rye, ryegrass, safflower, sorghum, soybean, strawberry, sugarcane, sudangrass, sunflower, sweet corn, switchgrass, timothy, tomato, and wheat. Increases in non-seed protein in such plants can provide improved nutritional content in edible fruits and vegetables, or improved animal forage. Decreases in non-seed protein can provide more efficient partitioning of nitrogen to plant part(s) that are harvested for human or animal consumption.

In some embodiments, a plant in which expression of a protein-modulating polypeptide having an amino acid sequence corresponding to SEQ ID NO:112, SEQ ID NO:130, or SEQ ID NO:141 is modulated can have modulated levels of seed oil accompanying increased levels of seed protein. The oil level can be modulated by at least 2 percent, e.g., 2, 3, 4, 5, 10, 15, 20, 25, 30, 35, or more than 35 percent.

In some embodiments, a plant in which expression of a protein-modulating polypeptide having an amino acid sequence corresponding to SEQ ID NO:80 or SEQ ID NO:84 is modulated can have increased levels of seed oil accompanying modulated levels of seed protein. The oil level can be increased by at least 2 percent, e.g., 2, 3, 4, 5, 6, 7, 8, 9, 10, 11, 12, 13, 14, 15, 16, 17, 18, 19, 20, 25, 30, 35, 40, 45, or more than 45 percent, as compared to the oil level in a corresponding control plant that does not express the transgene.

In some embodiments, a plant in which expression of a protein-modulating polypeptide having an amino acid sequence corresponding to SEQ ID NO:114 is modulated can have decreased levels of seed oil accompanying increased levels of seed protein. The oil level can be decreased by at least 4 percent, e.g., 5, 10, 15, 20, 25, 30, 35, or more than 35 percent, as compared to the oil level in a corresponding control plant that does not express the transgene.

Typically, a difference (e.g., an increase) in the amount of oil or protein in a transgenic plant or cell relative to a control plant or cell is considered statistically significant at p≦90.05 with an appropriate parametric or non-parametric statistic, e.g., Chi-square test, Student's t-test, Mann-Whitney test, or F-test. In some embodiments, a difference in the amount of oil or protein is statistically significant at p<0.01, p<0.005, or p<0.001. A statistically significant difference in, for example, the amount of protein in a transgenic plant compared to the amount in cells of a control plant indicates that (1) the recombinant nucleic acid present in the transgenic plant results in altered protein levels and/or (2) the recombinant nucleic acid warrants further study as a candidate for altering the amount of protein in a plant.

Information that the polypeptides disclosed herein can modulate protein content can be useful in breeding of crop plants. Based on the effect of disclosed polypeptides on protein content, one can search for and identify polymorphisms linked to genetic loci for such polypeptides. Polymorphisms that can be identified include simple sequence repeats (SSRs), rapid amplification of polymorphic DNA (RAPDs), amplified fragment length polymorphisms (AFLPs) and restriction fragment length polymorphisms (RFLPs).

If a polymorphism is identified, its presence and frequency in populations is analyzed to determine if it is statistically significantly correlated to an alteration in protein content. Those polymorphisms that are correlated with an alteration in protein content can be incorporated into a marker assisted breeding program to facilitate the development of lines that have a desired alteration in protein content. Typically, a polymorphism identified in such a manner is used with polymorphisms at other loci that are also correlated with a desired alteration in protein content.

### Articles of Manufacture

Transgenic plants provided herein have particular uses in the agricultural and nutritional industries. For example, transgenic plants described herein can be used to make animal feed and food products, such as grains and fresh, canned, and frozen vegetables. Suitable plants with which to make such products include alfalfa, barley, beans, clover, corn, millet, oat, peas, rice, rye, soybean, timothy, and wheat. For example, soybeans can be used to make various food products, including tofu, soy flour, and soy protein concentrates and isolates. Soy protein concentrates can be used to make textured soy protein products that resemble meat products. Soy protein isolates can be added to many soy food products, such as soy sausage patties, soybean burgers, soy protein bars, powdered soy protein beverages, soy protein baby formulas, and soy protein supplements. Such products are useful to provide increased or decreased protein and caloric content in the diet.

Seeds from transgenic plants described herein can be used as is, e.g., to grow plants, or can be used to make food products, such as flour. Seeds can be conditioned and bagged in packaging material by means known in the art to form an article of manufacture. Packaging material such as paper and cloth are well known in the art. A package of seed can have a label e.g., a tag or label secured to the packaging material, a label printed on the packaging material, or a label inserted within the package.

The invention will be further described in the following examples, which do not limit the scope of the invention described in the claims.

### EXAMPLES

### Example 1

### Transgenic Plants

The following symbols are used in the Examples: T₁: first generation transformant; T₂: second generation, progeny of self-pollinated T₁ plants; T₃: third generation, progeny of self-pollinated T₂ plants; T₄: fourth generation, progeny of self-pollinated T₃ plants. Independent transformations are referred to as events.

The following is a list of nucleic acids that were isolated from Arabidopsis thaliana plants. Ceres cDNA ID no. 7089429 (SEQ ID NO:83) is a genomic DNA clone that is predicted to encode a 360 amino acid geranylgeranyl pyrophosphate synthase polypeptide (genomic locus At3g14530; SEQ ID NO:84). Ceres CLONE ID no. 33780 (SEQ ID NO:79) is a cDNA clone that is predicted to encode a 158 amino acid polypeptide (genomic locus At4g21740; SEQ ID NO:80). Ceres cDNA ID no. 12720115 (SEQ ID NO:111) is a cDNA clone that is predicted to encode a 604 amino acid polypeptide containing a leucine rich repeat (genomic locus At2g35155; SEQ ID NO:112). Ceres cDNA ID no. 13579142 (SEQ ID NO:129) is a genomic DNA clone that is predicted to encode a 268 amino acid zinc knuckle polypeptide (genomic locus At5g52380; SEQ ID NO:130). Ceres CLONE ID no. 42577 (SEQ ID NO:101) is a cDNA clone that is predicted to encode a 172 amino acid polypeptide (genomic locus At5g41050; SEQ ID NO:102). Ceres CDNA ID no. 23416880 (SEQ ID NO:113) is a genomic DNA clone that is predicted to encode a 333 amino acid 3-phosphoinositide-dependent protein kinase-1 polypeptide (genomic locus At3g10572; SEQ ID NO:114). Ceres ANNOT ID no. 570373 (SEQ ID NO:174) is a DNA clone that is predicted to encode a 103 amino acid ribosomal polypeptide (SEQ ID NO:175). Ceres ANNOT ID no. 546661 (SEQ ID NO:170) is a DNA clone that is predicted to encode a 156 amino acid polypeptide (SEQ ID NO:171). Ceres ANNOT ID no. 543117 (SEQ ID NO:160) is a DNA clone that is predicted to encode a 622 amino acid kinase polypeptide (SEQ ID NO:161). Ceres CLONE ID no. 8161 (SEQ ID NO:204) is a DNA clone that is predicted to encode a 218 amino acid polypeptide (SEQ ID NO:205). Ceres CLONE ID no. 4595 (SEQ ID NO:179) is a DNA clone that is predicted to encode a 382 amino acid polypeptide containing an RNA recognition motif (SEQ ID NO:180). Ceres cDNA ID no. 36509475 (SEQ ID NO:208) is a DNA clone that is predicted to encode a 162 amino acid polypeptide (SEQ ID NO:209).

The following nucleic acid was isolated from Brassica napus. Ceres CLONE ID no. 1103471 (SEQ ID NO:140) is a cDNA clone that is predicted to encode a 189 amino acid polypeptide containing a zinc finger domain (SEQ ID NO:141).

The following nucleic acids were isolated from Zea mays. Ceres CLONE ID no. 285705 (SEQ ID NO:94) is a cDNA clone that is predicted to encode a 434 amino acid WD repeat polypeptide (SEQ ID NO:95). Ceres CLONE ID no. 400568 (SEQ ID NO:118) is a cDNA clone that is predicted to encode a 272 amino acid polypeptide (SEQ ID NO:119).

The following nucleic acids were isolated from Glycine max. Ceres cDNA ID no. 23698270 (SEQ ID NO:206) is a 370 nucleotide DNA clone. Ceres CLONE ID no. 531679 (SEQ ID NO:181) is a DNA clone that is predicted to encode a 251 amino acid polypeptide (SEQ ID NO:182). Ceres CLONE ID no. 558363 (SEQ ID NO:190) is a DNA clone that is predicted to encode a 392 amino acid glycosyl hydrolase family polypeptide (SEQ ID NO:191).

Each isolated nucleic acid described above was cloned into a Ti plasmid vector, CRS 338, containing a phosphinothricin acetyltransferase gene which confers Finale™ resistance to transformed plants. Constructs were made using CRS 338 that contained Ceres CDNA ID no. 7089429, Ceres CLONE ID no. 33780, Ceres CDNA ID no. 12720115, Ceres CDNA ID no. 13579142, Ceres CLONE ID no. 42577, Ceres CDNA ID no. 23416880, Ceres ANNOT ID no. 570373, Ceres ANNOT ID no. 546661, Ceres ANNOT ID no. 543117, Ceres CLONE ID no. 4595, Ceres CDNA ID no. 36509475, Ceres CLONE ID no. 1103471, Ceres CLONE ID no. 285705, Ceres CLONE ID no. 400568, Ceres CDNA ID no. 23698270, Ceres CLONE ID no. 531679, or Ceres CLONE ID no. 558363, each operably linked to a CaMV 35S promoter. A construct also was made using CRS 338 that contained Ceres CLONE ID no. 8161 operably linked to a p326F promoter. Wild-type Arabidopsis thaliana ecotype Wassilewskija (Ws) plants were transformed separately with each construct. The transformations were performed essentially as described in Bechtold et al., C.R. Acad. Sci. Paris, 316:1194-1199 (1993).

Transgenic Arabidopsis lines containing Ceres CDNA ID no. 7089429, Ceres CLONE ID no. 33780, Ceres CDNA ID no. 12720115, Ceres CDNA ID no. 13579142, Ceres CLONE ID no. 42577, Ceres CDNA ID no. 23416880, Ceres ANNOT ID no. 570373, Ceres ANNOT ID no. 546661, Ceres ANNOT ID no. 543117, Ceres CLONE ID no. 8161, Ceres CLONE ID no. 4595, Ceres CDNA ID no. 36509475, Ceres CLONE ID no. 1103471, Ceres CLONE ID no. 285705, Ceres CLONE ID no. 400568, Ceres CDNA ID no. 23698270, Ceres CLONE ID no. 531679, or Ceres CLONE ID no. 558363 were designated ME03761, ME02988, ME10006, ME12384, ME03537, ME11411, ME09083, ME10843, ME11388, ME12318, ME04921, ME10853, ME12636, ME07993, ME12151, ME08802, ME08800, or ME08803, respectively. The presence of each vector containing a Ceres clone described above in the respective transgenic Arabidopsis line transformed with the vector was confirmed by Finale™ resistance, polymerase chain reaction (PCR) amplification from green leaf tissue extract, and/or sequencing of PCR products. As controls, wild-type Arabidopsis ecotype Ws plants were transformed with the empty vector CRS 338.

### Example 2

### Analysis of Protein Content in Transgenic Arabidopsis Seeds

An analytical method based on Fourier transform near-infrared (FT-NIR) spectroscopy was developed, validated, and used to perform a high-throughput screen of transgenic seed lines for alterations in seed protein content. To calibrate the FT-NIR spectroscopy method, total nitrogen elemental analysis was used as a primary method to analyze a sub-population of randomly selected transgenic seed lines. The overall percentage of nitrogen in each sample was determined. Percent nitrogen values were multiplied by a conversion factor to obtain percent total protein values.

A conversion factor of 5.30 was selected based on data for cotton, sunflower, safflower, and sesame seed (Rhee, K. C., Determination of Total Nitrogen In Handbook of Food Analytical Chemistry—Water, Proteins, Enzymes, Lipids, and Carbohydrates (R. Wrolstad, et al., ed.), John Wiley and Sons, Inc., p. 105, (2005)). The same seed lines were then analyzed by FT-NIR spectroscopy, and the protein values calculated via the primary method were entered into the FT-NIR chemometrics software (Bruker Optics, Billerica, Mass.) to create a calibration curve for analysis of seed protein content by FT-NIR spectroscopy.

Elemental analysis was performed using a FlashEA 1112 NC Analyzer (Thermo Finnigan, San Jose, Calif.). To analyze total nitrogen content, 2.00±0.15 mg of dried transgenic Arabidopsis seed was weighed into a tared tin cup. The tin cup with the seed was weighed, crushed, folded in half, and placed into an autosampler slot on the FlashEA 1112 NC Analyzer (Thermo Finnigan). Matched controls were prepared in a manner identical to the experimental samples and spaced evenly throughout the batch. The first three samples in every batch were a blank (empty tin cup), a bypass, (approximately 5 mg of aspartic acid), and a standard (5.00±0.15 mg aspartic acid), respectively. Blanks were entered between every 15 experimental samples. Each sample was analyzed in triplicate.

The FlashEA 1112 NC Analyzer (Thermo Finnigan) instrument parameters were as follows: left furnace 900° C., right furnace 840° C., oven 50° C., gas flow carrier 130 mL/min., and gas flow reference 100 mL/min. The data parameter LLOD was 0.25 mg for the standard and different for other materials. The data parameter LLOQ was 3.0 mg for the standard, 1.0 mg for seed tissue, and different for other materials.

Quantification was performed using the Eager 300 software (Thermo Finnigan). Replicate percent nitrogen measurements were averaged and multiplied by a conversion factor of 5.30 to obtain percent total protein values. For results to be considered valid, the standard deviation between replicate samples was required to be less than 10%. The percent nitrogen of the aspartic acid standard was required to be within ±1.0% of the theoretical value. For a run to be declared valid, the weight of the aspartic acid (standard) was required to be between 4.85 and 5.15 mg, and the blank(s) were required to have no recorded nitrogen content.

The same seed lines that were analyzed for elemental nitrogen content were also analyzed by FT-NIR spectroscopy, and the percent total protein values determined by elemental analysis were entered into the FT-NIR chemometrics software (Bruker Optics, Billerica, Mass.) to create a calibration curve for protein content. The protein content of each seed line based on total nitrogen elemental analysis was plotted on the x-axis of the calibration curve. The y-axis of the calibration curve represented the predicted values based on the best-fit line. Data points were continually added to the calibration curve data set.

T₂ seed from each transgenic plant line was analyzed by FT-NIR spectroscopy. Sarstedt tubes containing seeds were placed directly on the lamp, and spectra were acquired through the bottom of the tube. The spectra were analyzed to determine seed protein content using the FT-NIR chemometrics software (Bruker Optics) and the protein calibration curve. Results for experimental samples were compared to population means and standard deviations calculated for transgenic seed lines that were planted within 30 days of the lines being analyzed and grown under the same conditions. Typically, results from three to four events of each of 400 to 1600 different transgenic lines were used to calculate a population mean. Each data point was assigned a z-score (z=(x−mean)/std), and a p-value was calculated for the z-score.

Transgenic seed lines with protein levels in T₂ seed that differed by more than two standard deviations from the population mean were selected for evaluation of protein levels in the T₃ generation. All events of selected lines were planted in individual pots. The pots were arranged randomly in flats along with pots containing matched control plants in order to minimize microenviroment effects. Matched control plants contained an empty version of the vector used to generate the transgenic seed lines. T₃ seed from up to five plants from each event was collected and analyzed individually using FT-NIR spectroscopy. Data from replicate samples were averaged and compared to controls using the Student's t-test.

### Example 3

### Analysis of Oil Content in Transgenic Arabidopsis Seeds

An analytical method based on Fourier transform near-infrared (FT-NIR) spectroscopy was developed, validated, and used to perform a high-throughput screen of transgenic seed lines for alterations in seed oil content. To calibrate the FT-NIR spectroscopy method, a sub-population of transgenic seed lines was randomly selected and analyzed for oil content using a direct primary method. Fatty acid methyl ester (FAME) analysis by gas chromatography-mass spectroscopy (GC-MS) was used as the direct primary method to determine the total fatty acid content for each seed line and produce the FT-NIR spectroscopy calibration curves for oil.

To analyze seed oil content using GC-MS, seed tissue was homogenized in liquid nitrogen using a mortar and pestle to create a powder. The tissue was weighed, and 5.0±0.25 mg were transferred into a 2 mL Eppendorf tube. The exact weight of each sample was recorded. One mL of 2.5% H₂SO₄ (v/v in methanol) and 20 μL of undecanoic acid internal standard (1 mg/mL in hexane) were added to the weighed seed tissue. The tubes were incubated for two hours at 90° C. in a pre-equilibrated heating block. The samples were removed from the heating block and allowed to cool to room temperature. The contents of each Eppendorf tube were poured into a 15 mL polypropylene conical tube, and 1.5 mL of a 0.9% NaCl solution and 0.75 mL of hexane were added to each tube. The tubes were vortexed for 30 seconds and incubated at room temperature for 15 minutes. The samples were then centrifuged at 4,000 rpm for 5 minutes using a bench top centrifuge. If emulsions remained, then the centrifugation step was repeated until they were dissipated. One hundred μL of the hexane (top) layer was pipetted into a 1.5 mL autosampler vial with minimum volume insert. The samples were stored no longer than 1 week at −80° C. until they were analyzed.

Samples were analyzed using a Shimadzu QP-2010 GC-MS (Shimadzu Scientific Instruments, Columbia, Md.). The first and last sample of each batch consisted of a blank (hexane). Every fifth sample in the batch also consisted of a blank. Prior to sample analysis, a 7-point calibration curve was generated using the Supelco 37 component FAME mix (0.00004 mg/mL to 0.2 mg/mL). The injection volume was 1 μL.

The GC parameters were as follows: column oven temperature: 70° C., inject temperature: 230° C., inject mode: split, flow control mode: linear velocity, column flow:1.0 mL/min, pressure:53.5 mL/min, total flow:29.0 mL/min, purge flow:3.0 mL/min, split ratio: 25.0. The temperature gradient was as follows: 70° C. for 5 minutes, increasing to 350° C. at a rate of 5 degrees per minute, and then held at 350° C. for 1 minute. The MS parameters were as follows: ion source temperature: 200° C., interface temperature: 240° C., solvent cut time: 2 minutes, detector gain mode: relative, detector gain: 0.6 kV, threshold: 1000, group: 1, start time: 3 minutes, end time: 62 minutes, ACQ mode: scan, interval: 0.5 second, scan speed: 666, start M/z: 40, end M/z: 350. The instrument was tuned each time the column was cut or a new column was used.

The data were analyzed using the Shimadzu GC-MS Solutions software. Peak areas were integrated and exported to an Excel spreadsheet. Fatty acid peak areas were normalized to the internal standard, the amount of tissue weighed, and the slope of the corresponding calibration curve generated using the FAME mixture. Peak areas were also multiplied by the volume of hexane (0.75 mL) used to extract the fatty acids.

The same seed lines that were analyzed using GC-MS were also analyzed by FT-NIR spectroscopy, and the oil values determined by the GC-MS primary method were entered into the FT-NIR chemometrics software (Bruker Optics, Billerica, Mass.) to create a calibration curve for oil content. The actual oil content of each seed line analyzed using GC-MS was plotted on the x-axis of the calibration curve. The y-axis of the calibration curve represented the predicted values based on the best-fit line. Data points were continually added to the calibration curve data set.

T₂ seed from each transgenic plant line was analyzed by FT-NIR spectroscopy. Sarstedt tubes containing seeds were placed directly on the lamp, and spectra were acquired through the bottom of the tube. The spectra were analyzed to determine seed oil content using the FT-NIR chemometrics software (Bruker Optics) and the oil calibration curve. Results for experimental samples were compared to population means and standard deviations calculated for transgenic seed lines that were planted within 30 days of the lines being analyzed and grown under the same conditions. Typically, results from three to four events of each of 400 to 1600 different transgenic lines were used to calculate a population mean. Each data point was assigned a z-score (z=(x−mean)/std), and a p-value was calculated for the z-score.

Transgenic seed lines with protein levels in T₂ seed that differed by more than two standard deviations from the population mean were also analyzed to determine oil levels in the T₃ generation. Events of selected lines were planted in individual pots. The pots were arranged randomly in flats along with pots containing matched control plants in order to minimize microenvironment effects. Matched control plants contained an empty version of the vector used to generate the transgenic seed lines. T₃ seed from up to five plants from each event was collected and analyzed individually using FT-NIR spectroscopy. Data from replicate samples were averaged and compared to controls using the Student's t-test.

### Example 4

### Results for ME03761 Events

T₂ and T₃ seed from five events of ME03761 containing Ceres CDNA ID no. 7089429 was analyzed for total protein content using FT-NIR spectroscopy as described in Example 2.

The protein content in T₂ seed from five events of ME03761 was significantly increased compared to the mean protein content in seed from transgenic Arabidopsis lines planted within 30 days of ME03761. As presented in Table 1, the protein content was increased to 124% in seed from events-01 and -04 and to 122%, 121%, and 136% in seed from events-02, -03, and -05, respectively, compared to the population mean.

TABLE 1
Protein content (% control) in T₂ and T₃ seed from
ME03761 events containing Ceres CDNA ID no. 7089429

| | Event - | Event - | Event - | Event - | Event - | |
|---|---|---|---|---|---|---|
| | 01 | 02 | 03 | 04 | 05 | Control |
| Protein content (% control) in | 124 | 122 | 121 | 124 | 136 | 100 ± 0* |
| T₂ seed | | | | | | |
| p-value | 0.02 | 0.03 | 0.03 | 0.02 | <0.01 | N/A |
| Protein content (% control) in | 102 ± 1 | 97 ± 2 | 108 ± 1 | 102 ± 1 | 106 ± 1 | 100 ± 1 |
| T₃ seed | | | | | | |
| p-value | 0.39 | 0.18 | <0.01 | 0.22 | 0.03 | N/A |
| No. of T₂ plants | 3 | 5 | 4 | 4 | 3 | 29 |

*Population mean of the protein content in seed from transgenic lines planted within 30 days of ME03761. Variation is presented as the standard error of the mean.

The protein content in T₃ seed from two events of ME03761 was significantly increased compared to the protein content in corresponding control seed. As presented in Table 1, the protein content was increased to 108% and 106% in seed from events-03 and -05, respectively, compared to the protein content in control seed.

T₂ and T₃ seed from five events of ME03761 containing Ceres cDNA ID no. 7089429 was also analyzed for total oil content using FT-NIR spectroscopy as described in Example 3.

The oil content in T₂ seed from ME03761 events was not observed to differ significantly from the mean oil content in seed from transgenic Arabidopsis lines planted within 30 days of ME03761 (Table 2).

TABLE 2
Oil content (% control) in T₂ and T₃ seed from
ME03761 events containing Ceres CDNA ID no. 7089429

| | Event - | Event - | Event - | Event - | Event - | |
|---|---|---|---|---|---|---|
| | 01 | 02 | 03 | 04 | 05 | Control |
| Oil content (% control) in T₂ | 94 | 89 | 96 | 101 | 90 | 100 ± 0* |
| seed | | | | | | |
| p-value | 0.37 | 0.15 | 0.48 | 0.56 | 0.17 | N/A |
| Oil content (% control) in T₃ | 101 ± 4 | 102 ± 1 | 104 ± 1 | 101 ± 2 | 102 ± 1 | 100 ± 1 |
| seed | | | | | | |
| p-value | 0.84 | 0.26 | 0.01 | 0.54 | 0.05 | N/A |
| No. of T₂ plants | 3 | 5 | 4 | 4 | 3 | 29 |

*Population mean of the oil content in seed from transgenic lines planted within 30 days of ME03761. Variation is presented as the standard error of the mean.

The oil content in T₃ seed from two events of ME03761 events was significantly increased compared to the oil content in corresponding control seed. As presented in Table 2, the oil content was increased to 104% and 102% in seed from events-03 and -05, respectively, compared to the oil content in control seed.

The physical appearances of T₁ ME03761 plants were similar to those of corresponding control plants. There were no observable or statistically significant differences between T₂ ME03761 and control plants in germination, onset of flowering, rosette area, fertility, and general morphology/architecture.

### Example 5

### Results for ME02988 Events

T₂ and T₃ seed from five events of ME02988 containing Ceres CLONE ID no. 33780 was analyzed for total protein content using FT-NIR spectroscopy as described in Example 2.

The protein content in T₂ seed from three events of ME02988 was significantly increased compared to the mean protein content in seed from transgenic Arabidopsis lines planted within 30 days of ME02988. As presented in Table 3, the protein content was increased to 128%, 119%, and 117% in seed from events-01, -03, and -04, respectively, compared to the population mean.

TABLE 3
Protein content (% control) in T₂ and T₃ seed
from ME02988 events containing Ceres CLONE ID no. 33780

| | Event - | Event - | Event - | Event - | Event - | |
|---|---|---|---|---|---|---|
| | 01 | 02 | 03 | 04 | 05 | Control |
| Protein content (% control) in | 128 | 113 | 119 | 117 | 110 | 100 ± 0* |
| T₂ seed | | | | | | |
| p-value | <0.01 | 0.10 | 0.02 | 0.03 | 0.20 | N/A |
| Protein content (% control) in | 108 ± 1 | 99 ± 1 | 104 ± 0 | 97 ± 1 | 96 ± 1 | 100 ± 1 |
| T₃ seed | | | | | | |
| p-value | <0.01 | 0.64 | <0.01 | 0.14 | <0.01 | N/A |
| No. of T₂ plants | 3 | 5 | 4 | 5 | 3 | 29 |

*Population mean of the protein content in seed from transgenic lines planted within 30 days of ME02988. Variation is presented as the standard error of the mean.

The protein content in T₃ seed from two events of ME02988 was significantly increased compared to the protein content in corresponding control seed. As presented in Table 3, the protein content was increased to 108% and 104% in seed from events-01 and 03, respectively, compared to the protein content in control seed. The protein content in T₃ seed from one event of ME02988 was significantly decreased compared to the protein content in corresponding control seed. As presented in Table 3, the protein content was decreased to 96% in seed from event-05 compared to the protein content in corresponding control seed.

T₂ and T₃ seed from five events of ME02988 containing Ceres CLONE ID no. 33780 was also analyzed for total oil content using FT-NIR spectroscopy as described in Example 3.

The oil content in T₂ seed from ME02988 events was not observed to differ significantly from the mean oil content in seed from transgenic Arabidopsis lines planted within 30 days of ME02988 (Table 4).

TABLE 4
Oil content (% control) in T₂ and T₃ seed
from ME02988 events containing Ceres CLONE ID no. 33780

| | Event - | Event - | Event - | Event - | Event - | |
|---|---|---|---|---|---|---|
| | 01 | 02 | 03 | 04 | 05 | Control |
| Oil content (% control) in T₂ | 106 | 105 | 98 | 100 | 98 | 100 ± 0* |
| seed | | | | | | |
| p-value | 0.36 | 0.42 | 0.63 | 0.67 | 0.64 | N/A |
| Oil content (% control) in T₃ | 100 ± 1 | 101 ± 1 | 103 ± 0 | 102 ± 1 | 98 ± 2 | 100 ± 1 |
| seed | | | | | | |
| p-value | 0.76 | 0.38 | <0.01 | 0.22 | 0.28 | N/A |
| No. of T₂ plants | 3 | 5 | 4 | 5 | 3 | 29 |

*Population mean of the oil content in seed from transgenic lines planted within 30 days of ME02988. Variation is presented as the standard error of the mean.

The oil content in T₃ seed from one event of ME02988 was significantly increased compared to the oil content in corresponding control seed. As presented in Table 4, the oil content was increased to 103% in seed from event-03 compared to the oil content in control seed.

The physical appearances of T₁ ME02988 plants were similar to those of corresponding control plants. There were no observable or statistically significant differences between T₂ ME02988 and control plants in germination, onset of flowering, rosette area, fertility, and general morphology/architecture.

### Example 6

### Results for ME10006 Events

T₂ and T₃ seed from five events of ME10006 containing Ceres CDNA ID no. 12720115 was analyzed for total protein content using FT-NIR spectroscopy as described in Example 2.

The protein content in T₂ seed from two events of ME10006 was significantly increased compared to the mean protein content of seed from transgenic Arabidopsis lines planted within 30 days of ME10006. As presented in Table 5, the protein content was increased to 162% and 141% in seed from events-01 and -02, respectively, compared to the population mean.

TABLE 5
Protein content (% control) in T₂ and T₃ seed
from ME10006 events containing Ceres CDNA ID no. 12720115

| | Event - | Event - | Event - | Event - | Event - | |
|---|---|---|---|---|---|---|
| | 01 | 02 | 03 | 04 | 05 | Control |
| Protein content (% control) in | 162 | 141 | 121 | 124 | 110 | 100 ± 0* |
| T₂ seed | | | | | | |
| p-value | <0.01 | 0.01 | 0.13 | 0.10 | 0.24 | N/A |
| Protein content (% control) in | 112 ± 1 | 107 ± 1 | 111 ± 1 | 111 ± 1 | 104 ± 3 | 100 ± 1 |
| T₃ seed | | | | | | |
| p-value | <0.01 | <0.01 | <0.01 | <0.01 | 0.22 | N/A |
| No. of T₂ plants | 5 | 5 | 5 | 5 | 5 | 15 |

*Population mean of the protein content in seed from transgenic lines planted within 30 days of ME10006. Variation is presented as the standard error of the mean.

The protein content in T₃ seed from four events of ME10006 was significantly increased compared to the protein content in corresponding control seed. As presented in Table 5, the protein content was increased to 112% and 107% in seed from events-01 and -02, respectively, and to 111% in seed from events-03, and -04 compared to the protein content in control seed.

T₂ and T₃ seed from five events of ME10006 containing Ceres CDNA ID no. 12720115 was also analyzed for total oil content using FT-NIR spectroscopy as described in Example 3. The oil content in T₂ seed from one event of ME10006 was significantly decreased compared to the mean oil content in seed from transgenic Arabidopsis lines planted within 30 days of ME10006. As presented in Table 6, the oil content was decreased to 80% in seed from event-01 compared to the population mean.

TABLE 6
Oil content (% control) in T₂ and T₃ seed from
ME10006 events containing Ceres CDNA ID no. 12720115

| | Event -01 | Event -02 | Event -03 | Event -04 | Event -05 | Control |
|---|---|---|---|---|---|---|
| Oil content (% control) in T₂ | 80 | 94 | 96 | 99 | 101 | 100 ± 0* |
| seed | | | | | | |
| p-value | 0.03 | 0.40 | 0.45 | 0.50 | 0.50 | N/A |
| Oil content (% control) in T₃ | 102 ± 1 | 102 ± 1 | 102 ± 1 | 102 ± 1 | 97 ± 1 | 100 ± 0 |
| seed | | | | | | |
| p-value | 0.18 | 0.04 | 0.30 | 0.04 | 0.03 | N/A |
| No. of T₂ plants | 5 | 5 | 5 | 5 | 5 | 15 |

*Population mean of the oil content in seed from transgenic lines planted within 30 days of ME10006. Variation is presented as the standard error of the mean.

The oil content in T₃ seed from one event of ME10006 was significantly decreased compared to the oil content in corresponding control seed. As presented in Table 6, the oil content was decreased to 97% in seed from event-05 compared to the oil content in corresponding control seed. The oil content in T₃ seed from two events of ME10006 was significantly increased compared to the oil content in corresponding control seed. As presented in Table 6, the oil content was increased to 102% in seed from events-02 and -04 compared to the oil content in control seed.

The physical appearances of T₁ ME10006 plants were similar to those of corresponding control plants. There were no observable or statistically significant differences between T₂ ME10006 and control plants in germination, onset of flowering, rosette area, fertility, and general morphology/architecture.

### Example 7

### Results for ME12384 Events

T₂ and T₃ seed from five events of ME12384 containing Ceres CDNA ID no. 13579142 was analyzed for total protein content using FT-NIR spectroscopy as described in Example 2.

The protein content in T₂ seed from three events of ME12384 was significantly increased compared to the mean protein content in seed from transgenic Arabidopsis lines planted within 30 days of ME12384. As presented in Table 7, the protein content was increased to 136%, 130%, and 129% in seed from events-01, -03, and -05, respectively, compared to the population mean.

TABLE 7
Protein content (% control) in T₂ and T₃ seed
from ME12384 events containing Ceres CDNA ID no. 13579142

| | Event - | Event - | Event - | Event - | Event - | |
|---|---|---|---|---|---|---|
| | 01 | 02 | 03 | 04 | 05 | Control |
| Protein content (% control) in | 136 | 109 | 130 | 108 | 129 | 100 ± 0* |
| T₂ seed | | | | | | |
| p-value | 0.02 | 0.23 | 0.04 | 0.25 | 0.05 | N/A |
| Protein content (% control) in | 112 | 113 ± 2 | 124 ± 1 | 108 ± 1 | 114 ± 2 | 100 ± 1 |
| T₃ seed | | | | | | |
| p-value | 0.01 | <0.01 | <0.01 | <0.01 | <0.01 | N/A |
| No. of T₂ plants | 1 | 3 | 3 | 5 | 4 | 15 |

*Population mean of the protein content in seed from transgenic lines planted within 30 days of ME12384. Variation is presented as the standard error of the mean.

The protein content in T₃ seed from five events of ME12384 was significantly increased compared to the protein content in corresponding control seed. As presented in Table 7, the protein content was increased to 112%, 113%, 124%, 108%, and 114% in seed from events-01, -02, -03, -04 and -05, respectively, compared to the protein content in control seed.

T₂ and T₃ seed from five events of ME12384 containing Ceres CDNA ID no. 13579142 was also analyzed for total oil content using FT-NIR spectroscopy as described in Example 3.

The oil content in T₂ seed from two events of ME12384 was significantly decreased compared to the mean oil content in seed from transgenic Arabidopsis lines planted within 30 days of ME12384. As presented in Table 8, the oil content was decreased to 79% and 78% in seed from events-01 and -03, respectively, compared to the population mean.

TABLE 8
Oil content (% control) in T₂ and T₃ seed
from ME12384 events containing Ceres CDNA ID no. 13579142

| | Event - | Event - | Event - | Event - | Event - | |
|---|---|---|---|---|---|---|
| | 01 | 02 | 03 | 04 | 05 | Control |
| Oil content (% control) in T₂ | 79 | 91 | 78 | 95 | 81 | 100 ± 0* |
| seed | | | | | | |
| p-value | 0.05 | 0.30 | 0.04 | 0.39 | 0.08 | N/A |
| Oil content (% control) in T₃ | 99 | 104 ± 2 | 100 ± 2 | 107 ± 2 | 109 ± 1 | 100 ± 0 |
| seed | | | | | | |
| p-value | 0.98 | 0.25 | 0.89 | 0.01 | <0.01 | N/A |
| No. of T₂ plants | 1 | 3 | 3 | 5 | 4 | 15 |

*Population mean of the oil content in seed from transgenic lines planted within 30 days of ME12384. Variation is presented as the standard error of the mean.

The oil content in T₃ seed from two events of ME12384 was significantly increased compared to the oil content in corresponding control seed. As presented in Table 8, the oil content was increased to 107% and 109% in seed from events-04 and -05, respectively, compared to the oil content in control seed.

The physical appearances of T₁ ME12384 plants were similar to those of corresponding control plants. There were no observable or statistically significant differences between T₂ ME12384 and control plants in germination, onset of flowering, rosette area, fertility, and general morphology/architecture.

### Example 8

### Results for ME12636 Events

T₂ and T₃ seed from five events and four events, respectively, of ME12636 containing Ceres CLONE ID no. 1103471 was analyzed for total protein content using FT-NIR spectroscopy as described in Example 2.

The protein content in T₂ seed from four events of ME12636 was significantly increased compared to the mean protein content in seed from transgenic Arabidopsis lines planted within 30 days of ME12636. As presented in Table 9, the protein content was increased to 132%, 133%, 136%, and 129% in seed from events-01, -02, -04, and -05, respectively, compared to the population mean.

TABLE 9
Protein content (% control) in T₂ and T₃ seed
from ME12636 events containing Ceres CLONE ID no. 1103471

| | Event - | Event - | Event - | Event - | Event - | |
|---|---|---|---|---|---|---|
| | 01 | 02 | 03 | 04 | 05 | Control |
| Protein content (% control) in | 132 | 133 | 127 | 136 | 129 | 100 ± 0* |
| T₂ seed | | | | | | |
| p-value | 0.03 | 0.02 | 0.06 | 0.02 | 0.04 | N/A |
| Protein content (% control) in | 107 ± 1 | No data | 111 ± 0 | 113 ± 2 | 115 ± 1 | 100 ± 1 |
| T₃ seed | | | | | | |
| p-value | <0.01 | No data | <0.01 | <0.01 | <0.01 | N/A |
| No. of T₂ plants | 5 | No data | 4 | 5 | 4 | 15 |

*Population mean of the protein content in seed from transgenic lines planted within 30 days of ME12636. Variation is presented as the standard error of the mean.

The protein content in T₃ seed from four events of ME12636 was significantly increased compared to the protein content in corresponding control seed. As presented in Table 9, the protein content was increased to 107%, 111%, 113%, and 115% in seed from events-01, -03, -04, and -05, respectively, compared to the protein content in control seed.

T₂ and T₃ seed from five events and four events, respectively, of ME12636 containing Ceres CLONE ID no. 1103471 was also analyzed for total oil content using FT-NIR spectroscopy as described in Example 3.

The oil content in T₂ seed from ME12636 events was not observed to differ significantly from the mean oil content in seed from transgenic Arabidopsis lines planted within 30 days of ME12636 (Table 10).

TABLE 10
Oil content (% control) in T₂ and T₃ seed
from ME12636 events containing Ceres CLONE ID no. 1103471

| | Event -01 | Event -02 | Event -03 | Event -04 | Event -05 | Control |
|---|---|---|---|---|---|---|
| Oil content (% control) in T₂ | 100 | 102 | 102 | 94 | 93 | 100 ± 0* |
| seed | | | | | | |
| p-value | 0.56 | 0.53 | 0.55 | 0.42 | 0.38 | N/A |
| Oil content (% control) in T₃ | 104 ± 1 | No data | 104 ± 1 | 101 ± 1 | 91 ± 2 | 100 ± 0 |
| seed | | | | | | |
| p-value | <0.01 | No data | 0.02 | 0.22 | <0.01 | N/A |
| No. of T₂ plants | 5 | No data | 4 | 5 | 4 | 15 |

*Population mean of the oil content in seed from transgenic lines planted within 30 days of ME12636. Variation is presented as the standard error of the mean.

The oil content in T₃ seed from two events of ME12636 was significantly increased compared to the oil content in corresponding control seed. As presented in Table 10, the oil content was increased to 104% in seed from events-01 and -03 compared to the oil content in control seed. The oil content in T₃ seed from one event of ME12636 was significantly decreased compared to the oil content in corresponding control seed. As presented in Table 10, the oil content was decreased to 91% in seed from event-05 compared to the oil content in control seed.

There were no observable or statistically significant differences between T₂ ME12636 and control plants in germination, onset of flowering, rosette area, fertility, and general morphology/architecture.

### Example 9

### Results for ME07993 Events

T₂ and T₃ seed from four events of ME07993 containing Ceres CLONE ID no. 285705 was analyzed for total protein content using FT-NIR spectroscopy as described in Example 2.

The protein content in T₂ seed from four events of ME07993 was significantly increased compared to the mean protein content in seed from transgenic Arabidopsis lines planted within 30 days of ME07993. As presented in Table 11, the protein content was increased to 139%, 134%, 138%, and 133% in seed from events-02, -03, -04, and -05, respectively, compared to the population mean.

TABLE 11
Protein content (% control) in T₂ and T₃ seed
from ME07993 events containing Ceres CLONE ID no. 285705

| | Event -02 | Event -03 | Event -04 | Event -05 | Control |
|---|---|---|---|---|---|
| Protein content (% control) in T₂ | 139 | 134 | 138 | 133 | 100 ± 0* |
| seed | | | | | |
| p-value | <0.01 | 0.02 | <0.01 | 0.02 | N/A |
| Protein content (% control) in T₃ | 104 ± 0 | 95 ± 2 | 101 ± 0 | 104 ± 1 | 100 ± 1 |
| seed | | | | | |
| p-value | <0.01 | 0.07 | 0.56 | 0.01 | N/A |
| No. of T₂ plants | 5 | 4 | 3 | 5 | 29 |

*Population mean of the protein content in seed from transgenic lines planted within 30 days of ME07993. Variation is presented as the standard error of the mean.

The protein content in T₃ seed from two events of ME07993 was significantly increased compared to the protein content in corresponding control seed. As presented in Table 11, the protein content was increased to 104% in seed from events-02 and -05 compared to the protein content in control seed.

T₂ and T₃ seed from four events of ME07993 containing Ceres CLONE ID no. 285705 was also analyzed for total oil content using FT-NIR spectroscopy as described in Example 3. The oil content in T₂ and T₃ seed from ME07993 events was not observed to differ significantly from the oil content in corresponding control seed (Table 12).

TABLE 12
Oil content (% control) in T₂ and T₃ seed
from ME07993 events containing Ceres CLONE ID no. 285705

| | Event -02 | Event -03 | Event -04 | Event -05 | Control |
|---|---|---|---|---|---|
| Oil content (% control) in T₂ seed | 100 | 102 | 97 | 106 | 100 ± 0* |
| p-value | 0.78 | 0.74 | 0.66 | 0.43 | N/A |
| Oil content (% control) in T₃ seed | 90 ± 4 | 99 ± 1 | 99 ± 1 | 101 ± 1 | 100 ± 1 |
| p-value | 0.06 | 0.71 | 0.59 | 0.48 | N/A |
| No. of T₂ plants | 5 | 4 | 3 | 5 | 29 |

*Population mean of the oil content in seed from transgenic lines planted within 30 days of ME07993. Variation is presented as the standard error of the mean.

The physical appearances of T₁ ME07993 plants were similar to those of corresponding control plants. There were no observable or statistically significant differences between T₂ ME07993 and control plants in germination, onset of flowering, rosette area, fertility, and general morphology/architecture.

### Example 10

### Results for ME03537 Events

T₂ and T₃ seed from five events of ME03537 containing Ceres CLONE ID no. 42577 was analyzed for total protein content using FT-NIR spectroscopy as described in Example 2.

The protein content in T₂ seed from three events of ME03537 was significantly increased compared to the mean protein content in seed from transgenic Arabidopsis lines planted within 30 days of ME03537. As presented in Table 13, the protein content was increased to 123%, 133%, and 127% in seed from events-02, -03, and -05, respectively, compared to the population mean.

TABLE 13
Protein content (% control) in T₂ and T₃ seed from ME03537
events containing Ceres CLONE ID no. 42577

| | Event | Event | Event | Event | Event | |
|---|---|---|---|---|---|---|
| | -01 | -02 | -03 | -04 | -05 | Control |
| Protein | 113 | 123 | 133 | 109 | 127 | 100 ± 0* |
| content (% | | | | | | |
| control) in | | | | | | |
| T₂ seed | | | | | | |
| p-value | 0.14 | 0.02 | <0.01 | 0.23 | 0.01 | N/A |
| Protein | 100 ± 1 | 101 ± 2 | 105 ± 1 | 107 ± 3 | 112 ± 1 | 100 ± 1 |
| content (% | | | | | | |
| control) in | | | | | | |
| T₃ seed | | | | | | |
| p-value | 0.87 | 0.83 | 0.01 | 0.18 | 0.04 | N/A |
| No. of T₂ | 2 | 5 | 4 | 3 | 2 | 29 or |
| plants | | | | | | 19** |

*Population mean of the protein content in seed from transgenic lines planted within 30 days of ME03537.
**For some events, 29 T₂ plants served as controls, and for the remaining events, 19 T₂ plants served as controls.
Variation is presented as the standard error of the mean.

The protein content in T₃ seed from two events of ME03537 was significantly increased compared to the protein content in corresponding control seed. As presented in Table 13, the protein content was increased to 105% and 112% in seed from events-03 and -05, respectively, compared to the protein content in control seed.

T₂ and T₃ seed from five events of ME03537 containing Ceres CLONE ID no. 42577 was also analyzed for total oil content using FT-NIR spectroscopy as described in Example 3. The oil content in T₂ and T₃ seed from ME03537 events was not observed to differ significantly from the oil content in corresponding control seed (Table 14).

TABLE 14
Oil content (% control) in T₂ and T₃ seed from ME03537
events containing Ceres CLONE ID no. 42577

| | Event | Event | Event | Event | Event | |
|---|---|---|---|---|---|---|
| | -01 | -02 | -03 | -04 | -05 | Control |
| Oil content | 94 | 90 | 94 | 95 | 91 | 100 ± 0* |
| (% control) | | | | | | |
| in T₂ seed | | | | | | |
| p-value | 0.40 | 0.17 | 0.36 | 0.46 | 0.21 | N/A |
| Oil content | 96 ± 2 | 102 ± 1 | 97 ± 2 | 94 ± 2 | 88 ± 4 | 100 ± 1 |
| (% control) | | | | | | |
| in T₃ seed | | | | | | |
| p-value | 0.25 | 0.17 | 0.18 | 0.09 | 0.14 | N/A |
| No. of | 2 | 5 | 4 | 3 | 2 | 29, 19 |
| T₂ plants | | | | | | |

*Population mean of the oil content in seed from transgenic lines planted within 30 days of ME12636.
Variation is presented as the standard error of the mean.

The physical appearances of T₁ ME03537 plants were similar to those of corresponding control plants. There were no observable or statistically significant differences between T₂ ME03537 and control plants in germination, onset of flowering, rosette area, fertility, and general morphology/architecture.

### Example 11

### Results for ME08802 Events

T₂ and T₃ seed from four events of ME08802 containing Ceres cDNA ID no. 23698270 was analyzed for total protein content using FT-NIR spectroscopy as described in Example 2.

The protein content in T₂ seed from three events of ME08802 was significantly increased compared to the mean protein content in seed from transgenic Arabidopsis lines planted within 30 days of ME08802. As presented in Table 15, the protein content was increased to 132%, 126%, and 123% in seed from events-01, -02, and -05, respectively, compared to the population mean.

TABLE 15
Protein content (% control) in T₂ and T₃ seed from ME08802
events containing Ceres CDNA ID no. 23698270

| | Event | | | | |
|---|---|---|---|---|---|
| | -01 | Event -02 | Event -04 | Event -05 | Control |
| Protein content | 132 | 126 | 121 | 123 | 100 ± 0* |
| (% control) | | | | | |
| in T₂ | | | | | |
| seed | | | | | |
| p-value | 0.01 | 0.03 | 0.07 | 0.05 | N/A |
| Protein content | 109 ± 0 | 105 ± 1 | 112 ± 3 | 120 ± 3 | 100 ± 1 |
| (% control) | | | | | |
| in T₃ | | | | | |
| seed | | | | | |
| p-value | <0.01 | <0.01 | 0.01 | <0.01 | N/A |
| No. of | 5 | 5 | 5 | 5 | 15 |
| T₂ plants | | | | | |

*Population mean of the protein content in seed from transgenic lines planted within 30 days of ME08802.
Variation is presented as the standard error of the mean.

The protein content in T₃ seed from four events of ME08802 was significantly increased compared to the protein content in corresponding control seed. As presented in Table 15, the protein content was increased to 109%, 105%, 112%, and 120% in seed from events-01, -02, -04, and -05, respectively, compared to the protein content in control seed.

T₂ and T₃ seed from four events of ME08802 containing Ceres CDNA ID no. 23698270 was also analyzed for total oil content using FT-NIR spectroscopy as described in Example 3. The oil content in T₂ and T₃ seed from ME08802 events was not observed to differ significantly from the oil content in corresponding control seed (Table 16).

TABLE 16
Oil content (% control) in T₂ and T₃ seed from ME08802
events containing Ceres CDNA ID no. 23698270

| | Event -01 | Event -02 | Event -04 | Event -05 | Control |
|---|---|---|---|---|---|
| Oil content | 102 | 102 | 99 | 96 | 100 ± 0* |
| (% control) | | | | | |
| in T₂ seed | | | | | |
| p-value | 0.69 | 0.68 | 0.71 | 0.60 | N/A |
| Oil content | 102 ± 1 | 102 ± 1 | 102 ± 2 | 100 ± 1 | 100 ± 0 |
| (% control) | | | | | |
| in T₃ seed | | | | | |
| p-value | 0.20 | 0.21 | 0.41 | 0.74 | N/A |
| No. of | 5 | 5 | 5 | 5 | 15 |
| T₂ plants | | | | | |

*Population mean of the oil content in seed from transgenic lines planted within 30 days of ME08802.
Variation is presented as the standard error of the mean.

The physical appearances of T₁ ME08802 plants were similar to those of corresponding control plants. There were no observable or statistically significant differences between T₂ ME08802 and control plants in germination, onset of flowering, rosette area, fertility, and general morphology/architecture.

### Example 12

### Results for ME1215 Events

T₂ and T₃ seed from five events and four events, respectively, of ME12151 containing Ceres CLONE ID no. 400568 was analyzed for total protein content using FT-NIR spectroscopy as described in Example 2.

The protein content in T₂ seed from five events of ME12151 was significantly increased compared to the mean protein content in seed from transgenic Arabidopsis lines planted within 30 days of ME12151. As presented in Table 17, the protein content was increased to 129% in seed from events-01 and -04, to 137% in seed from event-02, and to 131% in seed from events-03 and -05 compared to the population mean.

TABLE 17
Protein content (% control) in T₂ and T₃ seed from ME12151
events containing Ceres CLONE ID no. 400568

| | Event | Event | Event | Event | Event | |
|---|---|---|---|---|---|---|
| | -01 | -02 | -03 | -04 | -05 | Control |
| Protein | 129 | 137 | 131 | 129 | 131 | 100 ± 0* |
| content (% | | | | | | |
| control) in | | | | | | |
| T₂ seed | | | | | | |
| p-value | 0.04 | 0.01 | 0.03 | 0.05 | 0.04 | N/A |
| Protein | 109 ± 1 | 106 ± 1 | No data | 109 ± 1 | 108 ± 4 | 100 ± 1 |
| content (% | | | | | | |
| control) in | | | | | | |
| T₃ seed | | | | | | |
| p-value | <0.01 | 0.04 | No data | <0.01 | 0.24 | N/A |
| No. of | 5 | 2 | No data | 3 | 2 | 15 |
| T₂ plants | | | | | | |

*Population mean of the protein content in seed from transgenic lines planted within 30 days of ME12151.
Variation is presented as the standard error of the mean.

The protein content in T₃ seed from three events of ME12151 was significantly increased compared to the protein content in corresponding control seed. As presented in Table 17, the protein content was increased to 109% in seed from events-01 and -04 and to 106% in seed from event-02 compared to the protein content in control seed.

T₂ and T₃ seed from five events and four events, respectively, of ME12151 containing Ceres CLONE ID no. 400568 was also analyzed for total oil content using FT-NIR spectroscopy as described in Example 3. The oil content in T₂ and T₃ seed from ME12151 events was not observed to differ significantly from the oil content in corresponding control seed (Table 18).

TABLE 18
Oil content (% control) in T₂ and T₃ seed from ME12151
events containing Ceres CLONE ID no. 400568

| | Event | Event | Event | Event | Event | |
|---|---|---|---|---|---|---|
| | -01 | -02 | -03 | -04 | -05 | Control |
| Oil content | 91 | 85 | 85 | 81 | 87 | 100 ± 0* |
| (% control) | | | | | | |
| in T₂ | | | | | | |
| seed | | | | | | |
| p-value | 0.31 | 0.14 | 0.07 | 0.07 | 0.20 | N/A |
| Oil content | 99 ± 0 | 101 ± 0 | No data | 100 ± 1 | 102 ± 1 | 100 ± 0 |
| (% control) | | | | | | |
| in T₃ | | | | | | |
| seed | | | | | | |
| p-value | 0.18 | 0.15 | No data | 0.84 | 0.29 | N/A |
| No. of T₂ | 5 | 2 | No data | 3 | 2 | 15 |
| plants | | | | | | |

*Population mean of the oil content in seed from transgenic lines planted within 30 days of ME12151.
Variation is presented as the standard error of the mean.

The physical appearances of T₁ ME12151 plants were similar to those of corresponding control plants. There were no observable or statistically significant differences between T₂ ME12151 and control plants in germination, onset of flowering, rosette area, fertility, and general morphology/architecture.

### Example 13

### Results for ME11411 Events

T₂ and T₃ seed from four events of ME11411 containing Ceres CDNA ID no. 23416880 was analyzed for total protein content using FT-NIR spectroscopy as described in Example 2.

The protein content in T₂ seed from four events of ME11411 was significantly increased compared to the mean protein content in seed from transgenic Arabidopsis lines planted within 30 days of ME11411. As presented in Table 19, the protein content was increased to 135%, 139%, 136%, and 140% in seed from events-01, -02, -03, and -05, respectively, compared to the population mean.

TABLE 19
Protein content (% control) in T₂ and T₃ seed from ME11411
events containing Ceres CDNA ID no. 23416880

| | Event -01 | Event -02 | Event -03 | Event -05 | Control |
|---|---|---|---|---|---|
| Protein | 135 | 139 | 136 | 140 | 100 ± 0* |
| content (% | | | | | |
| control) in | | | | | |
| T₂ seed | | | | | |
| p-value | 0.04 | 0.02 | 0.03 | 0.02 | N/A |
| Protein | 103 ± 1 | 103 ± 1 | 105 ± 2 | 110 ± 2 | 100 ± 1 |
| content (% | | | | | |
| control) in | | | | | |
| T₃ seed | | | | | |
| p-value | 0.13 | 0.04 | 0.10 | 0.01 | N/A |
| No. of | 5 | 5 | 4 | 4 | 15 |
| T₂ plants | | | | | |

*Population mean of the protein content in seed from transgenic lines planted within 30 days of ME11411.
Variation is presented as the standard error of the mean.

The protein content in T₃ seed from two events of ME11411 was significantly increased compared to the protein content in corresponding control seed. As presented in Table 19, the protein content was increased to 103% and 110% in seed from events-02 and -05, respectively, compared to the protein content in control seed.

T₂ and T₃ seed from four events of ME11411 containing Ceres CDNA ID no. 23416880 was also analyzed for total oil content using FT-NIR spectroscopy as described in Example 3.

The oil content in T₂ seed from one event of ME11411 was significantly decreased compared to the mean oil content in seed from transgenic Arabidopsis lines planted within 30 days of ME11411. As presented in Table 20, the oil content was decreased to 80% in seed from event-01 compared to the population mean.

TABLE 20
Oil content (% control) in T₂ and T₃ seed from ME11411
events containing Ceres CDNA ID no. 23416880

| | Event -01 | Event -02 | Event -03 | Event -05 | Control |
|---|---|---|---|---|---|
| Oil content | 80 | 94 | 96 | 101 | 100 ± 0* |
| (% control) | | | | | |
| in T₂ seed | | | | | |
| p-value | 0.03 | 0.40 | 0.45 | 0.50 | N/A |
| Oil content | 101 ± 1 | 99 ± 1 | 99 ± 1 | 100 ± 1 | 100 ± 0 |
| (% control) | | | | | |
| in T₃ seed | | | | | |
| p-value | 0.31 | 0.57 | 0.67 | 0.83 | N/A |
| No. of | 5 | 5 | 4 | 4 | 15 |
| T₂ plants | | | | | |

*Population mean of the oil content in seed from transgenic lines planted within 30 days of ME11411.
Variation is presented as the standard error of the mean.

The oil content in T₃ seed from ME11411 events was not observed to differ significantly from the oil content in control seed (Table 20).

The physical appearances of T₁ ME11411 plants were similar to those of corresponding control plants. There were no observable or statistically significant differences between T₂ ME11411 and control plants in germination, onset of flowering, rosette area, fertility, and general morphology/architecture.

### Example 14

### Results for ME08800 Events

T₂ and T₃ seed from three events and five events, respectively, of ME08800 containing Ceres CLONE ID no. 531679 was analyzed for total protein content using FT-NIR spectroscopy as described in Example 2.

The protein content in T₂ seed from two events of ME08800 was significantly increased compared to the mean protein content in seed from transgenic Arabidopsis lines planted within 30 days of ME08800. As presented in Table 21, the protein content was increased to 128% and 122% in seed from events-01 and -05, respectively, compared to the population mean.

TABLE 21
Protein content (% control) in T₂ and T₃ seed from ME08800
events containing Ceres CLONE ID no. 531679

| | Event | Event | Event | Event | Event | |
|---|---|---|---|---|---|---|
| | -01 | -02 | -03 | -04 | -05 | Control |
| Protein | 128 | No data | No data | 116 | 122 | 100 ± 0* |
| content (% | | | | | | |
| control) in | | | | | | |
| T₂ seed | | | | | | |
| p-value | 0.02 | No data | No data | 0.13 | 0.05 | N/A |
| Protein | 117 ± 8 | 115 ± 6 | 122 ± 2 | 111 ± 4 | 114 ± 3 | 100 ± 0.01 |
| content (% | | | | | | |
| control) in | | | | | | |
| T₃ seed | | | | | | |
| p-value | 0.06 | <0.01 | <0.01 | <0.01 | <0.01 | N/A |

*Population mean of the protein content in seed from transgenic lines planted within 30 days of ME08800.
Variation is presented as the standard error of the mean.

The protein content in T₃ seed from four events of ME08800 was significantly increased compared to the protein content in corresponding control seed. As presented in Table 21, the protein content was increased to 115%, 122%, 111%, and 114% in seed from events-02, -03, -04, and -05, respectively, compared to the protein content in control seed.

### Example 15

### Results for ME08803 Events

T₂ and T₃ seed from three events and four events, respectively, of ME08803 containing Ceres CLONE ID no. 558363 was analyzed for total protein content using FT-NIR spectroscopy as described in Example 2.

The protein content in T₂ seed from three events of ME08803 was significantly increased compared to the mean protein content in seed from transgenic Arabidopsis lines planted within 30 days of ME08803. As presented in Table 22, the protein content was increased to 135% in seed from events-01 and -03 and to 124% in seed from event-04 compared to the population mean.

TABLE 22
Protein content (% control) in T₂ and T₃ seed from ME08803
events containing Ceres CLONE ID no. 558363

| | Event -01 | Event -02 | Event -03 | Event -04 | Control |
|---|---|---|---|---|---|
| Protein | 135 | No data | 135 | 124 | 100 ± 0* |
| content (% | | | | | |
| control) in | | | | | |
| T₂ seed | | | | | |
| p-value | <0.01 | No data | <0.01 | 0.04 | N/A |
| Protein | 101 ± 2 | 104 ± 2 | 109 ± 5 | 104 ± 4 | 100 ± 0.01 |
| content (% | | | | | |
| control) in | | | | | |
| T₃ seed | | | | | |
| p-value | 0.66 | 0.01 | 0.03 | 0.13 | N/A |

*Population mean of the protein content in seed from transgenic lines planted within 30 days of ME08803.
Variation is presented as the standard error of the mean.

The protein content in T₃ seed from two events of ME08803 was significantly increased compared to the protein content in corresponding control seed. As presented in Table 22, the protein content was increased to 104% and 109% in seed from events-02 and -03, respectively, compared to the protein content in control seed.

### Example 16

### Results for ME09083 Events

T₂ and T₃ seed from three events and four events, respectively, of ME09083 containing Ceres ANNOT ID no. 570373 was analyzed for total protein content using FT-NIR spectroscopy as described in Example 2.

The protein content in T₂ seed from three events of ME09083 was significantly increased compared to the mean protein content in seed from transgenic Arabidopsis lines planted within 30 days of ME09083. As presented in Table 23, the protein content was increased to 126%, 133%, and 125% in seed from events-01, -02, and -04, respectively, compared to the population mean.

TABLE 23
Protein content (% control) in T₂ and T₃ seed from ME09083
events containing Ceres ANNOT ID no. 570373

| | Event -01 | Event -02 | Event -03 | Event -04 | Control |
|---|---|---|---|---|---|
| Protein | 126 | 133 | No data | 125 | 100 ± 0* |
| content (% | | | | | |
| control) in | | | | | |
| T₂ seed | | | | | |
| p-value | 0.03 | <0.01 | No data | 0.03 | N/A |
| Protein | 98 ± 3 | 107 ± 4 | 103 ± 3 | 103 ± 7 | 100 ± 0.01 |
| content (% | | | | | |
| control) in | | | | | |
| T₃ seed | | | | | |
| p-value | 0.25 | 0.02 | 0.29 | 0.43 | N/A |

*Population mean of the protein content in seed from transgenic lines planted within 30 days of ME09083.
Variation is presented as the standard error of the mean.

The protein content in T₃ seed from one event of ME09083 was significantly increased compared to the protein content in corresponding control seed. As presented in Table 23, the protein content was increased to 107% in seed from event-02 compared to the protein content in control seed.

### Example 17

### Results for ME10843 Events

T₂ and T₃ seed from five events and three events, respectively, of ME10843 containing Ceres ANNOT ID no. 546661 was analyzed for total protein content using FT-NIR spectroscopy as described in Example 2.

The protein content in T₂ seed from five events of ME10843 was significantly increased compared to the mean protein content in seed from transgenic Arabidopsis lines planted within 30 days of ME10843. As presented in Table 24, the protein content was increased to 142%, 150%, 176%, 163%, and 150% in seed from events-01, -02, -03, -04, and -05, respectively, compared to the population mean.

TABLE 24
Protein content (% control) in T₂ and T₃ seed from ME10843
events containing Ceres ANNOT ID no. 546661

| | Event | Event | Event | Event | Event | |
|---|---|---|---|---|---|---|
| | -01 | -02 | -03 | -04 | -05 | Control |
| Protein | 142 | 150 | 176 | 163 | 150 | 100 ± 0* |
| content (% | | | | | | |
| control) in | | | | | | |
| T₂ seed | | | | | | |
| p-value | 0.02 | 0.01 | <0.01 | <0.01 | 0.01 | N/A |
| Protein | No data | 111 ± 6 | No data | 104 ± 2 | 101 ± 4 | 100 ± 0.01 |
| content (% | | | | | | |
| control) in | | | | | | |
| T₃ seed | | | | | | |
| p-value | No data | 0.21 | No data | 0.02 | 0.48 | N/A |

*Population mean of the protein content in seed from transgenic lines planted within 30 days of ME10843.
Variation is presented as the standard error of the mean.

The protein content in T₃ seed from one event of ME10843 was significantly increased compared to the protein content in corresponding control seed. As presented in Table 24, the protein content was increased to 104% in seed from event -04 compared to the protein content in control seed.

### Example 18

### Results for ME11388 Events

T₂ and T₃ seed from four events and five events, respectively, of ME11388 containing Ceres ANNOT ID no. 543117 was analyzed for total protein content using FT-NIR spectroscopy as described in Example 2.

The protein content in T₂ seed from four events of ME11388 was significantly increased compared to the mean protein content in seed from transgenic Arabidopsis lines planted within 30 days of ME11388. As presented in Table 25, the protein content was increased to 136% and 149% in seed from events-01 and -02, respectively, and to 141% in seed from events-03 and -05 compared to the population mean.

TABLE 25
Protein content (% control) in T₂ and T₃ seed from ME11388
events containing Ceres ANNOT ID no. 543117

| | Event | Event | Event | Event | Event | |
|---|---|---|---|---|---|---|
| | -01 | -02 | -03 | -04 | -05 | Control |
| Protein | 136 | 149 | 141 | No data | 141 | 100 ± 0* |
| content (% | | | | | | |
| control) in | | | | | | |
| T₂ seed | | | | | | |
| p-value | 0.04 | 0.01 | 0.02 | No data | 0.02 | N/A |
| Protein | 107 ± 8 | 103 ± 3 | 112 ± 2 | 111 ± 6 | 101 ± 2 | 100 ± 0.01 |
| content (% | | | | | | |
| control) in | | | | | | |
| T₃ seed | | | | | | |
| p-value | 0.12 | 0.20 | <0.01 | 0.06 | 0.35 | N/A |

*Population mean of the protein content in seed from transgenic lines planted within 30 days of ME11388.
Variation is presented as the standard error of the mean.

The protein content in T₃ seed from one event of ME11388 was significantly increased compared to the protein content in corresponding control seed. As presented in Table 25, the protein content was increased to 112% in seed from event-03 compared to the protein content in control seed.

### Example 19

### Results for ME12318 Events

T₂ and T₃ seed from five events and four events, respectively, of ME12318 containing Ceres CLONE ID no. 8161 was analyzed for total protein content using FT-NIR spectroscopy as described in Example 2.

The protein content in T₂ seed from four events of ME12318 was significantly increased compared to the mean protein content in seed from transgenic Arabidopsis lines planted within 30 days of ME12318. As presented in Table 26, the protein content was increased to 123%, 129%, 133%, and 130% in seed from events-01, -03, -04, and -05, respectively, compared to the population mean.

TABLE 26
Protein content (% control) in T₂ and T₃ seed from ME12318
events containing Ceres CLONE ID no. 8161

| | Event | Event | Event | Event | Event | |
|---|---|---|---|---|---|---|
| | -01 | -02 | -03 | -04 | -05 | Control |
| Protein | 123 | 120 | 129 | 133 | 130 | 100 ± 0* |
| content (% | | | | | | |
| control) in | | | | | | |
| T₂ seed | | | | | | |
| p-value | 0.05 | 0.08 | 0.02 | 0.01 | 0.01 | N/A |
| Protein | 127 | No data | 106 | 118 | 110 | 100 ± 0.01 |
| content (% | | | | | | |
| control) in | | | | | | |
| T₃ seed | | | | | | |
| p-value | <0.01 | No data | 0.30 | <0.01 | 0.05 | N/A |

*Population mean of the protein content in seed from transgenic lines planted within 30 days of ME12318.
Variation is presented as the standard error of the mean.

The protein content in T₃ seed from three events of ME12318 was significantly increased compared to the protein content in corresponding control seed. As presented in Table 26, the protein content was increased to 127%, 118%, and 110% in seed from events-01, -04, and -05, respectively, compared to the protein content in control seed.

### Example 20

### Results for ME04921 Events

T₂ and T₃ seed from four events and three events, respectively, of ME04921 containing Ceres CLONE ID no. 4595 was analyzed for total protein content using FT-NIR spectroscopy as described in Example 2.

The protein content in T₂ seed from four events of ME04921 was significantly increased compared to the mean protein content in seed from transgenic Arabidopsis lines planted within 30 days of ME04921. As presented in Table 27, the protein content was increased to 135% in seed from events-01 and -03, to 127% in seed from event-04, and to 138% in seed from event-05 compared to the population mean.

TABLE 27
Protein content (% control) in T₂ and T₃ seed from ME04921
events containing Ceres CLONE ID no. 4595

| | Event | Event | Event | Event | Event | |
|---|---|---|---|---|---|---|
| | -01 | -02 | -03 | -04 | -05 | Control |
| Protein | 135 | No data | 135 | 127 | 138 | 100 ± 0* |
| content (% | | | | | | |
| control) in | | | | | | |
| T₂ seed | | | | | | |
| p-value | 0.02 | No data | 0.02 | 0.05 | 0.01 | N/A |
| Protein | 96 ± 2 | 97 ± 5 | 104 ± 4 | No data | No data | 100 ± 0.01 |
| content (% | | | | | | |
| control) in | | | | | | |
| T₃ seed | | | | | | |
| p-value | 0.02 | 0.27 | 0.06 | No data | No data | N/A |

*Population mean of the protein content in seed from transgenic lines planted within 30 days of ME04921.
Variation is presented as the standard error of the mean.

The protein content in T₃ seed from one event of ME04921 was significantly decreased compared to the protein content in corresponding control seed. As presented in Table 27, the protein content was decreased to 96% in seed from event-01 compared to the protein content in control seed.

### Example 21

### Results for ME10853 Events

T₂ and T₃ seed from five events and three events, respectively, of ME10853 containing Ceres CDNA ID no. 36509475 was analyzed for total protein content using FT-NIR spectroscopy as described in Example 2.

The protein content in T₂ seed from three events of ME10853 was significantly increased compared to the mean protein content in seed from transgenic Arabidopsis lines planted within 30 days of ME10853. As presented in Table 28, the protein content was increased to 151%, 145%, and 153% in seed from events-01, -03, and -05, respectively, compared to the population mean.

TABLE 28
Protein content (% control) in T₂ and T₃ seed from ME10853 events containing Ceres
CDNA ID no. 36509475

| | Event | Event | Event | Event | Event | |
|---|---|---|---|---|---|---|
| | -01 | -02 | -03 | -04 | -05 | Control |
| Protein content (% control) in | 151 | 131 | 145 | 130 | 153 | 100 ± 0* |
| T₂ seed | | | | | | |
| p-value | 0.01 | 0.06 | 0.01 | 0.07 | <0.01 | N/A |
| Protein content (% control) in | 126 ± 6 | 125 ± 4 | 113 ± 11 | No data | No data | 100 ± 0.01 |
| T₃ seed | | | | | | |
| p-value | 0.01 | <0.01 | 0.10 | No data | No data | N/A |

*Population mean of the protein content in seed from transgenic lines planted within 30 days of ME10853. Variation is presented as the standard error of the mean.

The protein content in T₃ seed from two events of ME10853 was significantly increased compared to the protein content in corresponding control seed. As presented in Table 28, the protein content was increased to 126% and 125% in seed from events-01 and -02, respectively, compared to the protein content in control seed.

### Example 22

### Results for ME01238, ME01455, ME07326, ME06747, ME14188, ME23595, and ME29952 Events

The following is a list of nucleic acids that were isolated from Arabidopsis thaliana plants. Ceres CLONE ID no. 29678 (SEQ ID NO:302) is predicted to encode a 360 amino acid polypeptide (SEQ ID NO:87) that is a homolog of the polypeptide set forth in SEQ ID NO:84. Ceres CLONE ID no. 100141 (SEQ ID NO:287) is predicted to encode a 258 amino acid polypeptide (SEQ ID NO:184) that is a homolog and/or ortholog of the polypeptide set forth in SEQ ID NO:182. Ceres CLONE ID no. 3297 (SEQ ID NO:303) is predicted to encode a 294 amino acid polypeptide (SEQ ID NO:100) that is a homolog and/or ortholog of the polypeptide set forth in SEQ ID NO:95.

A nucleic acid referred to as Ceres CLONE ID no. 1619683 (SEQ ID NO:298) was isolated from Glycine max. Ceres CLONE ID no. 1619683 (SEQ ID NO:298) is predicted to encode a 233 amino acid polypeptide (SEQ ID NO:158) that is a homolog and/or ortholog of the polypeptide set forth in SEQ ID NO:141.

Each isolated nucleic acid described above was cloned into a Ti plasmid vector, CRS 338, containing a phosphinothricin acetyltransferase gene which confers Finale™ resistance to transformed plants. Constructs were made using CRS 338 that contained Ceres CLONE ID no. 29678, Ceres CLONE ID no. 100141, Ceres CLONE ID no. 3297, or Ceres CLONE ID no. 1619683, each operably linked to a CaMV 35S promoter. Constructs also were made using CRS 338 that contained Ceres CLONE ID no. 29678 operably linked to a p32449 promoter or a p326F promoter. Wild-type Arabidopsis thaliana ecotype Wassilewskija (Ws) plants were transformed separately with each construct. The transformations were performed essentially as described in Bechtold et al., C.R. Acad. Sci. Paris, 316:1194-1199 (1993).

Transgenic Arabidopsis lines containing Ceres CLONE ID no. 29678, Ceres CLONE ID no. 100141, Ceres CLONE ID no. 3297, or Ceres CLONE ID no. 1619683 operably linked to a CaMV 35S promoter were designated ME01455, ME07326, ME06747, or ME29952, respectively. A transgenic Arabidopsis line containing Ceres CLONE ID no. 29678 operably linked to a p32449 promoter was designated ME01238. Two different transgenic Arabidopsis lines, each containing Ceres CLONE ID no. 29678 operably linked to a 326F promoter, were designated ME14188 and ME23595. The presence of each vector containing a Ceres clone described above in the respective transgenic Arabidopsis line transformed with the vector was confirmed by Finale™ resistance, polymerase chain reaction (PCR) amplification from green leaf tissue extract, and/or sequencing of PCR products. As controls, wild-type Arabidopsis ecotype Ws plants were transformed with the empty vector CRS 338.

T₂ seed from events of each of ME01455, ME07326, ME06747, ME29952, ME01238, ME14188, and ME23595 was analyzed for total protein content using FT-NIR spectroscopy as described in Example 2. The results of the analyses were inconclusive.

### Example 23

### Determination of Functional Homolog and/or Ortholog Sequences

A subject sequence was considered a functional homolog or ortholog of a query sequence if the subject and query sequences encoded proteins having a similar function and/or activity. A process known as Reciprocal BLAST (Rivera et al., Proc. Natl. Acad. Sci. USA, 95:6239-6244 (1998)) was used to identify potential functional homolog and/or ortholog sequences from databases consisting of all available public and proprietary peptide sequences, including NR from NCBI and peptide translations from Ceres clones.

Before starting a Reciprocal BLAST process, a specific query polypeptide was searched against all peptides from its source species using BLAST in order to identify polypeptides having BLAST sequence identity of 80% or greater to the query polypeptide and an alignment length of 85% or greater along the shorter sequence in the alignment. The query polypeptide and any of the aforementioned identified polypeptides were designated as a cluster.

The BLASTP version 2.0 program from Washington University at Saint Louis, Mo., USA, was used to determine BLAST sequence identity and E-value. The BLASTP version 2.0 program includes the following parameters: 1) an E-value cutoff of 1.0e-5; 2) a word size of 5; and 3) the -postsw option. The BLAST sequence identity was calculated based on the alignment of the first BLAST HSP (High-scoring Segment Pairs) of the identified potential functional homolog and/or ortholog sequence with a specific query polypeptide. The number of identically matched residues in the BLAST HSP alignment was divided by the HSP length, and then multiplied by 100 to get the BLAST sequence identity. The HSP length typically included gaps in the alignment, but in some cases gaps were excluded.

The main Reciprocal BLAST process consists of two rounds of BLAST searches; forward search and reverse search. In the forward search step, a query polypeptide sequence, “polypeptide A,” from source species SA was BLASTed against all protein sequences from a species of interest. Top hits were determined using an E-value cutoff of 10⁻⁵ and a sequence identity cutoff of 35%. Among the top hits, the sequence having the lowest E-value was designated as the best hit, and considered a potential functional homolog or ortholog. Any other top hit that had a sequence identity of 80% or greater to the best hit or to the original query polypeptide was considered a potential functional homolog or ortholog as well. This process was repeated for all species of interest.

In the reverse search round, the top hits identified in the forward search from all species were BLASTed against all protein sequences from the source species SA. A top hit from the forward search that returned a polypeptide from the aforementioned cluster as its best hit was also considered as a potential functional homolog or ortholog.

Functional homologs and/or orthologs were identified by manual inspection of potential functional homolog and/or ortholog sequences. Representative functional homologs and/or orthologs for SEQ ID NO:80, SEQ ID NO:84, SEQ ID NO:95, SEQ ID NO:102, SEQ ID NO:114, SEQ ID NO:119, SEQ ID NO:130, SEQ ID NO:141, SEQ ID NO:161, SEQ ID NO:171, SEQ ID NO:175, SEQ ID NO:182, SEQ ID NO:191, and SEQ ID NO:209 are shown in FIGS. 1-14, respectively. The percent identities of functional homologs and/or orthologs to SEQ ID NO:80, SEQ ID NO:84, SEQ ID NO:95, SEQ ID NO:102, SEQ ID NO:114, SEQ ID NO:119, SEQ ID NO:130, SEQ ID NO:141, SEQ ID NO:161, SEQ ID NO:171, SEQ ID NO:175, SEQ ID NO:182, SEQ ID NO:191, and SEQ ID NO:209 are shown below in Tables 2942, respectively. The BLAST sequence identities and E-values given in Tables 2942 were taken from the forward search round of the Reciprocal BLAST process.

TABLE 29
Percent identity to Ceres CLONE ID no. 33780 (SEQ ID NO: 80)

| | | SEQ ID | % | | HMM bit |
|---|---|---|---|---|---|
| Designation | Species | NO: | Identity | e-value | score |
| Ceres CLONE | Arabidopsis | 80 | N/A | N/A | 411.9 |
| ID no. 33780 | thaliana | | | | |
| Ceres CLONE | Brassica | 81 | 80.9 | 7.40E−48 | 334.4 |
| ID no. 1082418 | napus | | | | |
| Ceres CLONE | Glycine max | 82 | 78.1 | 8.49E−47 | 354.2 |
| ID no. 1058516 | | | | | |
| Ceres CLONE | Gossypium | 224 | | | 396.4 |
| ID no. 1808721 | hirsutum | | | | |

TABLE 30
Percent identity to Ceres CDNA ID no. 7089429 (SEQ ID NO: 84)

| | | SEQ ID | % | | HMM bit |
|---|---|---|---|---|---|
| Designation | Species | NO: | Identity | e-value | score |
| Ceres CDNA ID no. | Arabidopsis | 84 | N/A | N/A | 713.5 |
| 7089429 | thaliana | | | | |
| Public GI no. | Arabidopsis | 85 | 94.1 | 1.60E−171 | 712 |
| 26450928 | thaliana | | | | |
| Ceres CLONE ID no. | Arabidopsis | 87 | 93.8 | 2.99E−170 | 705.1 |
| 29678 | thaliana | | | | |
| Public GI no. | Arabidopsis | 88 | 88.2 | 1.80E−156 | 702.9 |
| 11994525 | thaliana | | | | |
| Ceres CLONE ID no. | Arabidopsis | 89 | 88.2 | 1.80E−156 | |
| 117906 | thaliana | | | | |
| Public GI no. | Arabidopsis | 90 | 84.7 | 1.89E−152 | 689.6 |
| 50253560 | thaliana | | | | |
| Public GI no. | Arabidopsis | 91 | 84.4 | 8.19E−152 | 686.9 |
| 62320250 | thaliana | | | | |
| Public GI no. | Picrorhiza | 92 | 71 | 2.89E−110 | 800.8 |
| 58201026 | kurrooa | | | | |
| Public GI no. | Eucommia | 93 | 70.1 | 9.50E−103 | 740.1 |
| 14422402 | ulmoides | | | | |
| Ceres ANNOT ID no. | Populus | 214 | | | 794 |
| 1457156 | balsamifera | | | | |
| | subsp. | | | | |
| | trichocarpa | | | | |
| Ceres ANNOT ID no. | Populus | 217 | | | 801.2 |
| 1487885 | balsamifera | | | | |
| | subsp. | | | | |
| | trichocarpa | | | | |
| Ceres CLONE ID no. | Panicum | 226 | | | 718.4 |
| 1811354 | virgatum | | | | |
| Ceres CLONE ID no. | Gossypium | 240 | | | 794.8 |
| 1894727 | hirsutum | | | | |
| Ceres CLONE ID no. | Glycine max | 248 | | | 796.1 |
| 470181 | | | | | |
| Ceres CLONE ID no. | Triticum | 254 | | | 714 |
| 753701 | aestivum | | | | |
| Public GI ID no. | Oryza sativa | 257 | | | 721.6 |
| 115473007 | subsp. japonica | | | | |
| Public GI ID no. | Ostreococcus | 258 | | | 670.3 |
| 116060748 | tauri | | | | |
| Public GI ID no. | Capsicum | 259 | | | 769.2 |
| 121145 | annuum | | | | |
| Public GI ID no. | Catharanthus | 261 | | | 810.5 |
| 13431546 | roseus | | | | |
| Public GI ID no. | Sinapis alba | 262 | | | 785.8 |
| 13431547 | | | | | |
| Public GI ID no. | Abies grandis | 263 | | | 739.7 |
| 17352451 | | | | | |
| Public GI ID no. | Gentiana lutea | 264 | | | 700.3 |
| 18146809 | | | | | |
| Public GI ID no. | Cistus incanus | 265 | | | 778.2 |
| 20386368 | subsp. creticus | | | | |
| Public GI ID no. | Abies grandis | 266 | | | 720.7 |
| 22535957 | | | | | |
| Public GI ID no. | Ginkgo biloba | 267 | | | 756.5 |
| 34484306 | | | | | |
| Public GI ID no. | Helianthus | 268 | | | 759.4 |
| 3885426 | annuus | | | | |
| Public GI ID no. | Plectranthus | 269 | | | 769.2 |
| 41059107 | barbatus | | | | |
| Public GI ID no. | Taxus | 270 | | | 748.4 |
| 4322331 | canadensis | | | | |
| Public GI ID no. | Antirrhinum | 271 | | | 813.2 |
| 46241274 | majus | | | | |
| Public GI ID no. | Daucus carota | 272 | | | 747.3 |
| 4958918 | | | | | |
| Public GI ID no. | Adonis | 273 | | | 785.6 |
| 56122554 | palaestina | | | | |
| Public GI ID no. | Croton | 274 | | | 801.1 |
| 6277254 | sublyratus | | | | |
| Public GI ID no. | Scoparia dulcis | 275 | | | 790.4 |
| 6277256 | | | | | |
| Public GI ID no. | Mentha x | 276 | | | 780.5 |
| 6449052 | piperita | | | | |
| Public GI ID no. | Hevea | 277 | | | 804 |
| 75250205 | brasiliensis | | | | |
| Public GI ID no. | Daucus carota | 278 | | | 752.8 |
| 79154586 | subsp. sativus | | | | |
| Public GI ID no. | Solanum | 279 | | | 757.1 |
| 82547882 | lycopersicum | | | | |
| Public GI ID no. | Chrysanthemum | 280 | | | 802.5 |
| 87299435 | x morifolium | | | | |
| Public GI ID no. | Arnebia | 281 | | | 673.5 |
| 88910043 | euchroma | | | | |
| Public GI ID no. | Stevia | 282 | | | 776.8 |
| 90289577 | rebaudiana | | | | |
| Public GI ID no. | Medicago | 284 | | | 803.3 |
| 92868507 | truncatula | | | | |
| Public GI ID no. | Tagetes erecta | 286 | | | 805.2 |
| 9971808 | | | | | |

TABLE 31
Percent identity to Ceres CLONE ID no. 285705 (SEQ ID NO: 95)

| | | | | | HMM |
|---|---|---|---|---|---|
| | | SEQ ID | % | | bit |
| Designation | Species | NO: | Identity | e-value | score |
| Ceres CLONE ID no. | Zea mays | 95 | N/A | N/A | 1268.8 |
| 285705 | | | | | |
| Public GI no. | Oryza sativa subsp. | 96 | 78.9 | 7.59E−181 | 1259.1 |
| 50918655 | japonica | | | | |
| Ceres ANNOT ID no. | Populus balsamifera | 98 | 54 | 4.39E−105 | 1238.2 |
| 1505632 | subsp. trichocarpa | | | | |
| Public GI no. | Arabidopsis thaliana | 99 | 53.9 | 4.00E−111 | 1213.7 |
| 16323464 | | | | | |
| Ceres CLONE ID no. | Arabidopsis thaliana | 100 | 53.9 | 4.00E−111 | 619 |
| 3297 | | | | | |
| Ceres CLONE ID no. | Panicum virgatum | 228 | | | 1279.2 |
| 1812252 | | | | | |

TABLE 32
Percent identity to Ceres CLONE ID no. 42577 (SEQ ID NO: 102)

| | | | | | HMM |
|---|---|---|---|---|---|
| | | SEQ ID | % | | bit |
| Designation | Species | NO: | Identity | e-value | score |
| Ceres CLONE ID no. | Arabidopsis thaliana | 102 | N/A | N/A | 407.3 |
| 42577 | | | | | |
| Ceres CLONE ID no. | Zea mays | 103 | 71.3 | 7.50E−55 | 425.6 |
| 1439269 | | | | | |
| Ceres ANNOT ID no. | Populus balsamifera | 105 | 57.7 | 9.29E−41 | 381.5 |
| 1440825 | subsp. trichocarpa | | | | |
| Ceres ANNOT ID no. | Populus balsamifera | 107 | 54.6 | 5.59E−43 | 438.7 |
| 1493706 | subsp. trichocarpa | | | | |
| Ceres ANNOT ID no. | Populus balsamifera | 109 | 52.7 | 1.29E−41 | 397.9 |
| 1485758 | subsp. trichocarpa | | | | |
| Ceres CLONE ID no. | Glycine max | 110 | 49.3 | 4.00E−40 | 408.9 |
| 645909 | | | | | |
| Ceres CLONE ID no. | Gossypium hirsutum | 230 | | | 424.1 |
| 1834121 | | | | | |
| Ceres CLONE ID no. | Gossypium hirsutum | 234 | | | 399.3 |
| 1838785 | | | | | |

TABLE 33
Percent identity to Ceres CDNA ID no. 23416880 (SEQ ID NO: 114)

| | | | | | HMM |
|---|---|---|---|---|---|
| | | SEQ ID | % | | bit |
| Designation | Species | NO: | Identity | e-value | score |
| Ceres CDNA ID no. | Arabidopsis thaliana | 114 | N/A | N/A | 921.2 |
| 23416880 | | | | | |
| Ceres ANNOT ID no. | Populus balsamifera | 116 | 49 | 1.09E−69 | 1047.3 |
| 1453934 | subsp. trichocarpa | | | | |
| Ceres CLONE ID no. | Glycine max | 117 | 45.7 | 3.19E−56 | 968.9 |
| 512894 | | | | | |

TABLE 34
Percent identity to Ceres CLONE ID no. 400568 (SEQ ID NO: 119)

| | | | | | HMM |
|---|---|---|---|---|---|
| | | SEQ ID | % | | bit |
| Designation | Species | NO: | Identity | e-value | score |
| Ceres CLONE ID no. | Zea mays | 119 | N/A | N/A | 599.2 |
| 400568 | | | | | |
| Ceres CLONE ID no. | Zea mays | 120 | 92.5 | 2.00E−132 | 579.2 |
| 1549251 | | | | | |
| Public GI no. | Oryza sativa subsp. | 121 | 88.3 | 3.20E−127 | 618.4 |
| 37718893 | japonica | | | | |
| Ceres CLONE ID no. | Triticum aestivum | 122 | 65.5 | 2.60E−84 | 591.9 |
| 937503 | | | | | |
| Ceres ANNOT ID no. | Populus balsamifera | 124 | 65.2 | 3.69E−85 | 616.5 |
| 1503141 | subsp. trichocarpa | | | | |
| Ceres CLONE ID no. | Glycine max | 125 | 65 | 5.29E−86 | 628 |
| 625275 | | | | | |
| Ceres CLONE ID no. | Glycine max | 126 | 64.1 | 7.99E−83 | 577.3 |
| 1371622 | | | | | |
| Ceres CLONE ID no. | Glycine max | 127 | 64.1 | 7.99E−83 | 575.7 |
| 511038 | | | | | |
| Public GI no. | Arabidopsis thaliana | 128 | 60.3 | 1.80E−76 | 573.1 |
| 11994767 | | | | | |
| Ceres CLONE ID no. | Panicum virgatum | 220 | | | 599.1 |
| 1719600 | | | | | |
| Ceres CLONE ID no. | Gossypium hirsutum | 232 | | | 620.1 |
| 1838546 | | | | | |
| Ceres CLONE ID no. | Gossypium hirsutum | 238 | | | 410 |
| 1845447 | | | | | |
| Ceres CLONE ID no. | Gossypium hirsutum | 242 | | | 475.6 |
| 1935338 | | | | | |

TABLE 35
Percent identity to Ceres CDNA ID no. 13579142 (SEQ ID NO: 130)

| | | | | | HMM |
|---|---|---|---|---|---|
| | | SEQ ID | % | | bit |
| Designation | Species | NO: | Identity | e-value | score |
| Ceres CDNA ID no. | Arabidopsis thaliana | 130 | N/A | N/A | 692.6 |
| 13579142 | | | | | |
| Ceres ANNOT ID no. | Populus balsamifera | 132 | 63.3 | 3.69E−91 | 695.8 |
| 1522260 | subsp. trichocarpa | | | | |
| Ceres CLONE ID no. | Glycine max | 133 | 63.1 | 5.09E−88 | 668.8 |
| 625135 | | | | | |
| Ceres ANNOT ID no. | Populus balsamifera | 135 | 62.1 | 3.79E−89 | 636.4 |
| 1527806 | subsp. trichocarpa | | | | |
| Ceres CLONE ID no. | Glycine max | 136 | 62.1 | 2.79E−87 | 629.6 |
| 463860 | | | | | |
| Public GI no. | Oryza sativa subsp. | 137 | 60.7 | 1.10E−78 | 748 |
| 50927857 | japonica | | | | |
| Ceres CLONE ID no. | Triticum aestivum | 138 | 56.2 | 1.80E−76 | 702.5 |
| 843076 | | | | | |
| Ceres CLONE ID no. | Zea mays | 139 | 53.7 | 8.70E−61 | 667.2 |
| 296774 | | | | | |
| Ceres CLONE ID no. | Panicum virgatum | 244 | | | 731.3 |
| 1999828 | | | | | |

TABLE 36
Percent identity to Ceres CLONE ID no. 1103471 (SEQ ID NO: 141)

| | | | | | HMM |
|---|---|---|---|---|---|
| | | SEQ ID | % | | bit |
| Designation | Species | NO: | Identity | e-value | score |
| Ceres CLONE ID no. | Brassica napus | 141 | N/A | N/A | 292.4 |
| 1103471 | | | | | |
| Public GI no. | Arabidopsis thaliana | 142 | 75.6 | 8.49E−63 | 279.6 |
| 21618143 | | | | | |
| Public GI no. 6009889 | Arabidopsis thaliana | 143 | 75.1 | 1.09E−62 | 278.3 |
| Public GI no. 4666360 | Datisca glomerata | 144 | 65.9 | 2.49E−40 | 432.6 |
| Public GI no. | Nicotiana | 145 | 65.5 | 1.19E−38 | 458.6 |
| 33771374 | benthamiana | | | | |
| Public GI no. 439493 | Petunia x hybrida | 146 | 65.3 | 1.39E−37 | 455.1 |
| Public GI no. | Nicotiana tabacum | 147 | 65 | 1.59E−38 | 460.8 |
| 71979887 | | | | | |
| Public GI no. | Capsicum annuum | 148 | 64.1 | 1.79E−37 | 449.4 |
| 33331578 | | | | | |
| Ceres CLONE ID no. | Glycine max | 149 | 63.5 | 8.10E−42 | 402.9 |
| 1240096 | | | | | |
| Public GI no. 7228329 | Medicago sativa | 150 | 63.3 | 5.09E−40 | 404.9 |
| | subsp. x varia | | | | |
| Ceres ANNOT ID no. | Populus balsamifera | 152 | 63.2 | 5.09E−40 | 443 |
| 1496702 | subsp. trichocarpa | | | | |
| Ceres ANNOT ID no. | Populus balsamifera | 154 | 63.2 | 5.09E−40 | 443 |
| 1443763 | subsp. trichocarpa | | | | |
| Public GI no. | Medicago truncatula | 155 | 62.5 | 2.29E−37 | 411.2 |
| 32441471 | | | | | |
| Ceres ANNOT ID no. | Populus balsamifera | 157 | 62.5 | 6.79E−38 | 446.8 |
| 1470888 | subsp. trichocarpa | | | | |
| Ceres CLONE ID no. | Glycine max | 158 | 62.2 | 3.20E−40 | 388.1 |
| 1619683 | | | | | |
| Public GI no. | Catharanthus roseus | 159 | 61.4 | 2.29E−37 | 421.6 |
| 55734108 | | | | | |

TABLE 37
Percent identity to Ceres ANNOT ID no. 543117 (SEQ ID NO: 161)

| | | | | | HMM |
|---|---|---|---|---|---|
| | | SEQ ID | % | | bit |
| Designation | Species | NO: | Identity | e-value | score |
| Ceres ANNOT ID no. | Arabidopsis thaliana | 161 | N/A | N/A | 1365.3 |
| 543117 | | | | | |
| Public GI no. | Arabidopsis thaliana | 162 | 80.7 | 1.10E−252 | 1328.2 |
| 20198186 | | | | | |
| Ceres ANNOT ID no. | Populus balsamifera | 164 | 72.4 | 1.10E−227 | 1457 |
| 1464138 | subsp. trichocarpa | | | | |
| Ceres ANNOT ID no. | Populus balsamifera | 166 | 71.1 | 3.70E−225 | 1388.4 |
| 1512068 | subsp. trichocarpa | | | | |
| Ceres CLONE ID no. | Glycine max | 167 | 70.7 | 2.39E−207 | 1435.5 |
| 481263 | | | | | |
| Public GI no. | Oryza sativa subsp. | 168 | 64.5 | 7.69E−186 | 1347.1 |
| 50929499 | japonica | | | | |
| Public GI no. | Oryza sativa subsp. | 169 | 62.5 | 6.20E−177 | 1124.9 |
| 50726629 | japonica | | | | |
| Ceres CLONE ID no. | Panicum virgatum | 222 | | | 1363.4 |
| 1806767 | | | | | |
| Ceres CLONE ID no. | Zea mays | 246 | | | 1352.3 |
| 378258 | | | | | |
| Public GI ID no. | Cleome spinosa | 283 | | | 1310.1 |
| 90657540 | | | | | |
| Public GI ID no. | Medicago truncatula | 285 | | | 1452.6 |
| 92894700 | | | | | |

TABLE 38
Percent identity to Ceres ANNOT ID no. 546661 (SEQ ID NO: 171)

| | | | | | HMM |
|---|---|---|---|---|---|
| | | SEQ ID | % | | bit |
| Designation | Species | NO: | Identity | e-value | score |
| Ceres ANNOT ID no. | Arabidopsis thaliana | 171 | N/A | N/A | 447.9 |
| 546661 | | | | | |
| Ceres ANNOT ID no. | Populus balsamifera | 173 | 52.7 | 2.89E−28 | 490.7 |
| 1467926 | subsp. trichocarpa | | | | |

TABLE 39
Percent identity to Ceres ANNOT ID no. 570373 (SEQ ID NO: 175)

| | | SEQ ID | % | | HMM bit |
|---|---|---|---|---|---|
| Designation | Species | NO: | Identity | e-value | score |
| Ceres ANNOT | Arabidopsis | 175 | N/A | N/A | 312.5 |
| ID no. 570373 | thaliana | | | | |
| Ceres CLONE | Glycine | 176 | 58.3 | 1.80E−21 | 296 |
| ID no. 1607448 | max | | | | |
| Ceres CLONE | Glycine | 177 | 57.8 | 5.89E−21 | 258.4 |
| ID no. 1043684 | max | | | | |
| Ceres CLONE | Glycine | 178 | 56.25 | 7.59E−21 | 254.5 |
| ID no. 723341 | max | | | | |

TABLE 40
Percent identity to Ceres CLONE ID no. 531679 (SEQ ID NO: 182)

| | | | | | HMM |
|---|---|---|---|---|---|
| | | SEQ ID | % | | bit |
| Designation | Species | NO: | Identity | e-value | score |
| Ceres CLONE ID no. | Glycine max | 182 | N/A | N/A | 647.6 |
| 531679 | | | | | |
| Ceres CLONE ID no. | Arabidopsis thaliana | 184 | 70.5 | 2.29E−90 | 633.9 |
| 100141 | | | | | |
| Ceres CLONE ID no. | Triticum aestivum | 185 | 68.5 | 3.49E−87 | 650.4 |
| 1054809 | | | | | |
| Public GI no. | Solanum tuberosum | 186 | 68.1 | 4.99E−88 | 640.6 |
| 78191452 | | | | | |
| Ceres CLONE ID no. | Zea mays | 187 | 60.6 | 5.09E−63 | 608.9 |
| 244926 | | | | | |
| Ceres ANNOT ID no. | Populus balsamifera | 189 | 77.2 | 4.00E−99 | 622.4 |
| 1586846 | subsp. trichocarpa | | | | |
| Ceres CLONE ID no. | Gossypium hirsutum | 236 | | | 678.1 |
| 1841382 | | | | | |
| Public GI ID no. | Oryza sativa subsp. | 260 | | | 639.1 |
| 125563536 | indica | | | | |

TABLE 41
Percent identity to Ceres CLONE ID no. 558363 (SEQ ID NO: 191)

| | | | | | HMM |
|---|---|---|---|---|---|
| | | SEQ ID | % | | bit |
| Designation | Species | NO: | Identity | e-value | score |
| Ceres CLONE ID no. | Glycine max | 191 | N/A | N/A | 1045.1 |
| 558363 | | | | | |
| Public GI no. 3413322 | Medicago sativa | 192 | 74.6 | 5.49E−160 | 1052.3 |
| Ceres CLONE ID no. | Glycine max | 193 | 73.4 | 6.99E−153 | 866.2 |
| 522929 | | | | | |
| Public GI no. | Medicago truncatula | 194 | 72.1 | 1.09E−129 | 862.9 |
| 41529571 | | | | | |
| Public GI no. | Medicago sativa | 195 | 69.7 | 1.90E−143 | 817.9 |
| 29123382 | | | | | |
| Public GI no. 668998 | Medicago sativa | 196 | 66.9 | 4.80E−138 | 791 |
| Ceres ANNOT ID no. | Populus balsamifera | 198 | 57.9 | 1.00E−96 | 823.6 |
| 1540806 | subsp. trichocarpa | | | | |
| Public GI no. 6714530 | Salix gilgiana | 199 | 55.6 | 1.40E−115 | 1044.3 |
| Public GI no. | Turnera subulata | 200 | 55.2 | 9.79E−115 | 1008.3 |
| 27902548 | | | | | |
| Public GI no. 6714526 | Salix gilgiana | 201 | 55 | 2.00E−114 | 1022.9 |
| Public GI no. 6714524 | Salix gilgiana | 202 | 54.3 | 2.59E−114 | 1012.9 |
| Public GI no. 6714528 | Salix gilgiana | 203 | 53.8 | 4.70E−115 | 1018.7 |

TABLE 42
Percent identity to Ceres CDNA ID no. 36509475 (SEQ ID NO: 209)

| | | | | | HMM |
|---|---|---|---|---|---|
| | | SEQ ID | % | | bit |
| Designation | Species | NO: | Identity | e-value | score |
| Ceres CDNA ID no. | Arabidopsis thaliana | 209 | N/A | N/A | 477.2 |
| 36509475 | | | | | |
| Ceres ANNOT ID no. | Populus balsamifera | 211 | 58.5 | 9.30E−43 | 515.1 |
| 1497025 | subsp. trichocarpa | | | | |
| Ceres CLONE ID no. | Glycine max | 212 | 56.2 | 7.20E−36 | 486.1 |
| 1659056 | | | | | |

### Example 24

### Generation of Hidden Markov Models

Hidden Markov Models (HMMs) were generated by the program HMMER 2.3.2 using groups of sequences as input that are homologous and/or orthologous to each of SEQ ID NO:80, SEQ ID NO:84, SEQ ID NO:95, SEQ ID NO:102, SEQ ID NO:114, SEQ ID NO:119, SEQ ID NO:130, SEQ ID NO:141, SEQ ID NO:161, SEQ ID NO:171, SEQ ID NO:175, SEQ ID NO:182, SEQ ID NO:191, SEQ ID NO:209, and SEQ ID NO:112. To generate each HMM, the default HMMER 2.3.2 program parameters configured for glocal alignments were used.

An HMM was generated using the sequences aligned in FIG. 1 as input. When fitted to the HMM, the sequences had the HMM bit scores listed in Table 29.

An HMM was generated using the sequences aligned in FIG. 2 as input. When fitted to the HMM, the sequences had the HMM bit scores listed in Table 30. Other homologous and/or orthologous sequences also were fitted to the HMM, and these sequences are listed in Table 30 along with their corresponding HMM bit scores.

An HMM was generated using the sequences aligned in FIG. 3 as input. When fitted to the HMM, the sequences had the HMM bit scores listed in Table 31. Another homologous and/or orthologous sequence (SEQ ID NO:100) also was fitted to the HMM, and this sequences is listed in Table 31 along with its corresponding HMM bit score.

An HMM was generated using the sequences aligned in FIG. 4 as input. When fitted to the HMM, the sequences had the HMM bit scores listed in Table 32. Other homologous and/or orthologous sequences also were fitted to the HMM, and these sequences are listed in Table 32 along with their corresponding HMM bit scores.

An HMM was generated using the sequences aligned in FIG. 5 as input. When fitted to the HMM, the sequences had the HMM bit scores listed in Table 33.

An HMM was generated using the sequences aligned in FIG. 6 as input. When fitted to the HMM, the sequences had the HMM bit scores listed in Table 34. Other homologous and/or orthologous sequences also were fitted to the HMM, and these sequences are listed in Table 34 along with their corresponding HMM bit scores.

An HMM was generated using the sequences aligned in FIG. 7 as input. When fitted to the HMM, the sequences had the HMM bit scores listed in Table 35. Other homologous and/or orthologous sequences also were fitted to the HMM, and these sequences are listed in Table 35 along with their corresponding HMM bit scores.

An HMM was generated using the sequences aligned in FIG. 8 as input. When fitted to the HMM, the sequences had the HMM bit scores listed in Table 36. Other homologous and/or orthologous sequences also were fitted to the HMM, and these sequences are listed in Table 36 along with their corresponding HMM bit scores.

An HMM was generated using the sequences aligned in FIG. 9 as input. When fitted to the HMM, the sequences had the HMM bit scores listed in Table 37. Other homologous and/or orthologous sequences also were fitted to the HMM, and these sequences are listed in Table 37 along with their corresponding HMM bit scores.

An HMM was generated using the sequences aligned in FIG. 10 as input. When fitted to the HMM, the sequences had the HMM bit scores listed in Table 38.

An HMM was generated using the sequences aligned in FIG. 11 as input. When fitted to the HMM, the sequences had the HMM bit scores listed in Table 39. Other homologous and/or orthologous sequences also were fitted to the HMM, and these sequences are listed in Table 39 along with their corresponding HMM bit scores.

An HMM was generated using the sequences aligned in FIG. 12 as input. When fitted to the HMM, the sequences had the HMM bit scores listed in Table 40. Another homologous and/or orthologous sequence (SEQ ID NO:184) also were fitted to the HMM, and this sequence is listed in Table 40 along with its corresponding HMM bit score.

An HMM was generated using the sequences aligned in FIG. 13 as input. When fitted to the HMM, the sequences had the HMM bit scores listed in Table 41. Other homologous and/or orthologous sequences also were fitted to the HMM, and these sequences are listed in Table 41 along with their corresponding HMM bit scores.

An HMM was generated using the sequences aligned in FIG. 14 as input. When fitted to the HMM, the sequences had the HMM bit scores listed in Table 42.

An HMM was generated using the sequences aligned in FIG. 15 as input. When fitted to the HMM, the sequences had the HMM bit scores listed in Table 43. Other homologous and/or orthologous sequences also were fitted to the HMM, and these sequences are listed in Table 43 along with their corresponding HMM bit scores.

TABLE 43
HMM bit scores of sequences related to SEQ ID NO: 349

| | | SEQ ID | HMM bit |
|---|---|---|---|
| Designation | Species | NO: | score |
| Ceres LOCUS ID no. | Arabidopsis | 349 | 1456.4 |
| At2g35155_FL | thaliana | | |
| Ceres ANNOT ID no. 1527550 | Populus | 315 | 1513.4 |
| | balsamifera | | |
| | subsp. | | |
| | trichocarpa | | |
| Ceres ANNOT ID no. 1537493 | Populus | 317 | 1416.9 |
| | balsamifera | | |
| | subsp. | | |
| | trichocarpa | | |
| Public GI ID no. 38344253 | Oryza sativa | 318 | 1487.2 |
| | subsp. japonica | | |
| Public GI ID no. 115476358 | Oryza sativa | 319 | 1281.5 |
| | subsp. japonica | | |
| Public GI ID no. 124359654 | Medicago | 320 | 1531.9 |
| | truncatula | | |
| Public GI ID no. 125561508 | Oryza sativa | 321 | 1281 |
| | subsp. indica | | |

An HMM was generated using the sequences aligned in FIG. 16 as input. When fitted to the HMM, the sequences had the HMM bit scores listed in Table 44. Other homologous and/or orthologous sequences also were fitted to the HMM, and these sequences are listed in Table 44 along with their corresponding HMM bit scores.

TABLE 44
HMM bit scores of sequences related to SEQ ID NO: 348

| | | SEQ ID | HMM bit |
|---|---|---|---|
| Designation | Species | NO: | score |
| Ceres LOCUS ID no. | Artificial | 348 | 641.5 |
| At2g35155_T | Sequence | | |
| Public GI ID no. 125561508_T | Artificial | 323 | 610.3 |
| | sequence | | |
| Public GI ID no. 115476358_T | Artificial | 324 | 607 |
| | Sequence | | |
| Ceres ANNOT ID no. | Artificial | 325 | 649.6 |
| 1527550_T | sequence | | |
| Ceres ANNOT ID no. | Artificial | 326 | 633.2 |
| 1537493_T | sequence | | |
| Public GI ID no. 124359654_T | Artificial | 327 | 627.1 |
| | sequence | | |
| Public GI ID no. 38344253_T | Artificial | 328 | 476.6 |
| | sequence | | |

An HMM was generated using the sequences aligned in FIG. 17 as input. When fitted to the HMM, the sequences had the HMM bit scores listed in Table 45. Other homologous and/or orthologous sequences also were fitted to the HMM, and these sequences are listed in Table 45 along with their corresponding HMM bit scores.

TABLE 45
HMM bit scores of sequences related to SEQ ID NO: 337

| | | SEQ | |
|---|---|---|---|
| | | ID | HMM bit |
| Designation | Species | NO: | score |
| Ceres LOCUS ID no. | Arabidopsis | 337 | 1388.9 |
| At1g78230_FL | thaliana | | |
| Ceres ANNOT ID no. 1451858 | Populus | 330 | 1700.7 |
| | balsamifera subsp. | | |
| | trichocarpa | | |
| Ceres CLONE ID no. 1574720 | Zea mays | 332 | 1475.5 |
| Ceres CLONE ID no. 1862739 | Panicum virgatum | 334 | 1396.2 |
| Ceres CLONE ID no. 546776 | Glycine max | 336 | 1673.6 |
| Ceres CLONE ID no. 1407377 | Zea mays | 339 | 954.2 |
| Ceres CLONE ID no. 1813489 | Panicum virgatum | 341 | 1214.7 |
| Ceres CLONE ID no. 1928737 | Gossypium | 343 | 1726.7 |
| | hirsutum | | |
| Public GI ID no. 115481758 | Oryza sativa subsp. | 344 | 1548.4 |
| | japonica | | |
| Public GI ID no. 125574597 | Oryza sativa subsp. | 345 | 1402.7 |
| | japonica | | |

An HMM was generated using the sequences aligned in FIG. 18 as input. When fitted to the HMM, the sequences had the HMM bit scores listed in Table 46. Other homologous and/or orthologous sequences also were fitted to the HMM, and these sequences are listed in Table 46 along with their corresponding HMM bit scores.

TABLE 46
HMM bit scores of sequences related to SEQ ID NO: 256

| | | SEQ | |
|---|---|---|---|
| | | ID | HMM bit |
| Designation | Species | NO: | score |
| Ceres LOCUS ID no. | Artificial sequence | 256 | 840.1 |
| At1g78230_T | | | |
| Ceres GI ID no. 115481758_T | Artificial Sequence | 183 | 816.8 |
| Ceres GI ID no. 125574597_T | Artificial sequence | 215 | 694 |
| Ceres CLONE ID no. | Artificial sequence | 218 | 601.7 |
| 1407377_T | | | |
| Ceres CLONE ID no. | Artificial sequence | 249 | 882.2 |
| 1813489_T | | | |
| Ceres CLONE ID no. | Artificial sequence | 250 | 572.6 |
| 1862739_T | | | |
| Ceres CLONE ID no. | Artificial sequence | 252 | 924.1 |
| 546776_T | | | |
| Ceres ANNOT ID no. | Artificial sequence | 346 | 965.8 |
| 1451858_T | | | |
| Ceres CLONE ID no. | Artificial sequence | 347 | 858.3 |
| 1574720_T | | | |
| Ceres CLONE ID no. | Artificial sequence | 86 | 955.1 |
| 1928737_T | | | |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-34. (canceled)

35. A plant cell comprising an exogenous nucleic acid, said exogenous nucleic acid comprising a nucleotide sequence encoding a polypeptide, wherein the HMM bit score of the amino acid sequence of said polypeptide is greater than 50, said HMM based on the amino acid sequences depicted in one of FIGS. 1-18, and wherein a tissue of a plant produced from said plant cell has a difference in the level of protein as compared to the corresponding level in tissue of a control plant that does not comprise said nucleic acid.

36. A plant cell comprising an exogenous nucleic acid, said exogenous nucleic acid comprising a nucleotide sequence encoding a polypeptide 208-257 amino acids in length, wherein said polypeptide is the amino terminus of a polypeptide having at least 500 amino acids and having an HMM bit score greater than 712, said HMM based on the amino acid sequences depicted in FIG. 15, and wherein a tissue of a plant produced from said plant cell has a difference in the level of protein as compared to the corresponding level in tissue of a control plant that does not comprise said nucleic acid.

37. A plant cell comprising an exogenous nucleic acid, said exogenous nucleic acid comprising a nucleotide sequence encoding a polypeptide 330-430 amino acids in length, wherein said polypeptide is the carboxy terminus of a polypeptide having at least 500 amino acids and having an HMM bit score greater than 724, said HMM based on the amino acid sequences depicted in FIG. 17, and wherein a tissue of a plant produced from said plant cell has a difference in the level of protein as compared to the corresponding level in tissue of a control plant that does not comprise said nucleic acid.

38. A plant cell comprising an exogenous nucleic acid, said exogenous nucleic acid comprising a nucleotide sequence encoding a polypeptide having 80 percent or greater sequence identity to an amino acid sequence selected from the group consisting of SEQ ID NOs:80-82, SEQ ID NOs:84-93, SEQ ID NOs:95-96, SEQ ID NOs:98-100, SEQ ID NOs:102-103, SEQ ID NO:105, SEQ ID NO:107, SEQ ID NOs:109-110, SEQ ID NO:112, SEQ ID NO:114, SEQ ID NOs:116-117, SEQ ID NOs:119-122, SEQ ID NOs:124-128, SEQ ID NO:130, SEQ ID NOs:132-133, SEQ ID NOs:135-139, SEQ ID NOs:141-150, SEQ ID NO:152, SEQ ID NOs:154-155, SEQ ID NOs:157-159, SEQ ID NOs:161-162, SEQ ID NO:164, SEQ ID NOs:166-169, SEQ ID NO:171, SEQ ID NO:173, SEQ ID NOs:175-178, SEQ ID NO:180, SEQ ID NOs:182-187, SEQ ID NO:189, SEQ ID NOs:191-196, SEQ ID NOs:198-203, SEQ ID NO:205, SEQ ID NO:209, SEQ ID NOs:211-212, SEQ ID NOs:214-215, SEQ ID NOs:217-218, SEQ ID NO:220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NOs:248-250, SEQ ID NO:252, SEQ ID NO:254, SEQ ID NOs:256-286, SEQ ID NO:315, SEQ ID NOs:317-328, SEQ ID NOs:330, SEQ ID NO:332, SEQ ID NO:334, SEQ ID NOs:336-337, SEQ ID NO:339, SEQ ID NO:341, and SEQ ID NOs:343-349, wherein a tissue of a plant produced from said plant cell has a difference in the level of protein as compared to the corresponding level in tissue of a control plant that does not comprise said nucleic acid.

39. A plant cell comprising an exogenous nucleic acid, said exogenous nucleic acid comprising a nucleotide sequence having 80 percent or greater sequence identity to a nucleotide sequence selected from the group consisting of SEQ ID NO:79, SEQ ID NO:83, SEQ ID NO:94, SEQ ID NO:97, SEQ ID NO:101, SEQ ID NO:104, SEQ ID NO:106, SEQ ID NO:108, SEQ ID NO:111, SEQ ID NO:113, SEQ ID NO:115, SEQ ID NO:118, SEQ ID NO:123, SEQ ID NO:129, SEQ ID NO:131, SEQ ID NO:134, SEQ ID NO:140, SEQ ID NO:151, SEQ ID NO:153, SEQ ID NO:156, SEQ ID NO:160, SEQ ID NO:163, SEQ ID NO:165, SEQ ID NO:170, SEQ ID NO:172, SEQ ID NO:174, SEQ ID NO:179, SEQ ID NO:181, SEQ ID NO:188, SEQ ID NO:190, SEQ ID NO:197, SEQ ID NO:204, SEQ ID NO:206, SEQ ID NO:208, SEQ ID NO:210, SEQ ID NO:213, SEQ ID NO:216, SEQ ID NO:219, SEQ ID NO:221, SEQ ID NO:223, SEQ ID NO:225, SEQ ID NO:227, SEQ ID NO:229, SEQ ID NO:231, SEQ ID NO:233, SEQ ID NO:235, SEQ ID NO:237, SEQ ID NO:239, SEQ ID NO:241, SEQ ID NO:243, SEQ ID NO:245, SEQ ID NO:247, SEQ ID NO:251, SEQ ID NO:253, SEQ ID NO:255, SEQ ID NOs:287-314, SEQ ID NO:316, SEQ ID NO:329, SEQ ID NO:331, SEQ ID NO:333, SEQ ID NO:335, SEQ ID NO:338, SEQ ID NO:340, and SEQ ID NO:342, wherein a tissue of a plant produced from said plant cell has a difference in the level of protein as compared to the corresponding level in tissue of a control plant that does not comprise said nucleic acid.

40. A plant cell comprising an exogenous nucleic acid, said exogenous nucleic acid comprising a regulatory region operably linked to a polynucleotide whose transcription product is at least 30 nucleotides in length and is complementary to a nucleic acid encoding a polypeptide, wherein the HMM bit score of the amino acid sequence of said polypeptide is greater than 50, said HMM based on the amino acid sequences depicted in one of FIGS. 1-18, wherein said regulatory region modulates transcription of said polynucleotide in said plant cell, and wherein a tissue of a plant produced from said plant cell has a difference in the level of protein as compared to the corresponding level in tissue of a control plant that does not comprise said nucleic acid.

41. A plant cell comprising an exogenous nucleic acid, said exogenous nucleic acid comprising a regulatory region operably linked to a polynucleotide that is transcribed into an interfering RNA effective for inhibiting expression of a polypeptide having 80 percent or greater sequence identity to an amino acid sequence selected from the group consisting of SEQ ID NOs:80-82, SEQ ID NOs:84-93, SEQ ID NOs:95-96, SEQ ID NOs:98-100, SEQ ID NOs:102-103, SEQ ID NO:105, SEQ ID NO:107, SEQ ID NOs:109-110, SEQ ID NO:112, SEQ ID NO:114, SEQ ID NOs:116-117, SEQ ID NOs:119-122, SEQ ID NOs:124-128, SEQ ID NO:130, SEQ ID NOs:132-133, SEQ ID NOs:135-139, SEQ ID NOs:141-150, SEQ ID NO:152, SEQ ID NOs:154-155, SEQ ID NOs:157-159, SEQ ID NOs:161-162, SEQ ID NO:164, SEQ ID NOs:166-169, SEQ ID NO:171, SEQ ID NO:173, SEQ ID NOs:175-178, SEQ ID NO:180, SEQ ID NOs:182-187, SEQ ID NO:189, SEQ ID NOs:191-196, SEQ ID NOs:198-203, SEQ ID NO:205, SEQ ID NO:209, SEQ ID NOs:211-212, SEQ ID NOs:214-215, SEQ ID NOs:217-218, SEQ ID NO:220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NOs:248-250, SEQ ID NO:252, SEQ ID NO:254, SEQ ID NOs:256-286, SEQ ID NO:315, SEQ ID NOs:317-328, SEQ ID NOs:330, SEQ ID NO:332, SEQ ID NO:334, SEQ ID NOs:336-337, SEQ ID NO:339, SEQ ID NO:341, and SEQ ID NOs:343-349, wherein said regulatory region modulates transcription of said polynucleotide in said plant cell, and wherein a tissue of a plant produced from said plant cell has a difference in the level of protein as compared to the corresponding level in tissue of a control plant that does not comprise said nucleic acid.

42. The plant cell of claim 35, wherein said plant is a dicot.

43. The plant cell of claim 42, wherein said plant is a species selected from the group consisting of Beta vulgaris (sugarbeet), Brassica napus (canola), Glycine max (soybean), Helianthus annuus (sunflower), Lupinus albus (lupin), and Medicago sativa (alfalfa).

44. The plant cell of claim 35, wherein said plant is a monocot.

45. The plant cell of claim 44 wherein said plant is a species selected from the group consisting of Oryza sativa (rice), Pennisetum glaucum (pearl millet), Triticum aestivum, (wheat), and Zea mays (corn).

46. The plant cell of claim 35, wherein said tissue is seed tissue.

47. A transgenic plant comprising the plant cell of claim 35.

48. Progeny of the plant of claim 47, wherein said progeny has a difference in the level of protein as compared to the level of protein in a corresponding control plant that does not comprise said exogenous nucleic acid.

49. Seed from a transgenic plant according to claim 47.

50. Vegetative tissue from a transgenic plant according to claim 47.

51. Fruit from a transgenic plant according to claim 47.

52. An isolated nucleic acid comprising a nucleotide sequence having 95% or greater sequence identity to a nucleotide sequence selected from the group consisting of SEQ ID NO:79, SEQ ID NO:83, SEQ ID NO:94, SEQ ID NO:97, SEQ ID NO:101, SEQ ID NO:104, SEQ ID NO:106, SEQ ID NO:108, SEQ ID NO:111, SEQ ID NO:113, SEQ ID NO:115, SEQ ID NO:118, SEQ ID NO:123, SEQ ID NO:129, SEQ ID NO:131, SEQ ID NO:134, SEQ ID NO:140, SEQ ID NO:151, SEQ ID NO:153, SEQ ID NO:156, SEQ ID NO:160, SEQ ID NO:163, SEQ ID NO:165, SEQ ID NO:170, SEQ ID NO:172, SEQ ID NO:174, SEQ ID NO:179, SEQ ID NO:181, SEQ ID NO:188, SEQ ID NO:190, SEQ ID NO:197, SEQ ID NO:204, SEQ ID NO:206, SEQ ID NO:208, SEQ ID NO:210, SEQ ID NO:213, SEQ ID NO:216, SEQ ID NO:219, SEQ ID NO:221, SEQ ID NO:223, SEQ ID NO:225, SEQ ID NO:227, SEQ ID NO:229, SEQ ID NO:231, SEQ ID NO:233, SEQ ID NO:235, SEQ ID NO:237, SEQ ID NO:239, SEQ ID NO:241, SEQ ID NO:243, SEQ ID NO:245, SEQ ID NO:247, SEQ ID NO:251, SEQ ID NO:253, SEQ ID NO:255, SEQ ID NOs:287-314, SEQ ID NO:316, SEQ ID NO:329, SEQ ID NO:331, SEQ ID NO:333, SEQ ID NO:335, SEQ ID NO:338, SEQ ID NO:340, and SEQ ID NO:342.

53. An isolated nucleic acid comprising a nucleotide sequence encoding a polypeptide having 80% or greater sequence identity to an amino acid sequence selected from the group consisting of SEQ ID NOs:80-82, SEQ ID NO:84, SEQ ID NO:89, SEQ ID NO:95, SEQ ID NO:98, SEQ ID NO:100, SEQ ID NOs:102-103, SEQ ID NO:105, SEQ ID NO:107, SEQ ID NOs:109-110, SEQ ID NO:112, SEQ ID NO:114, SEQ ID NOs:116-117, SEQ ID NOs:119-120, SEQ ID NO:122, SEQ ID NOs:124-127, SEQ ID NO:130, SEQ ID NOs:132-133, SEQ ID NOs:135-136, SEQ ID NOs:138-139, SEQ ID NO:141, SEQ ID NO:149, SEQ ID NO:152, SEQ ID NO:154, SEQ ID NOs:157-158, SEQ ID NO:161, SEQ ID NO:164, SEQ ID NOs:166-167, SEQ ID NO:171, SEQ ID NO:173, SEQ ID NOs:175-178, SEQ ID NO:180, SEQ ID NOs:182-185, SEQ ID NO:187, SEQ ID NO:189, SEQ ID NO:191, SEQ ID NO:193, SEQ ID NO:198, SEQ ID NO:205, SEQ ID NO:209, SEQ ID NOs:211-212, SEQ ID NOs:214-215, SEQ ID NOs:217-218, SEQ ID NO:220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NOs:248-250, SEQ ID NO:252, SEQ ID NO:254, SEQ ID NO:256, SEQ ID NO:315, SEQ ID NO:317, SEQ ID NOs:322, SEQ ID NOs:325-326, SEQ ID NO:330, SEQ ID NO:332, SEQ ID NO:334, SEQ ID NOs:336-337, SEQ ID NO:339, SEQ ID NO:341, SEQ ID NO:343, and SEQ ID NOs:346-349.
